SEQUENCE LISTING (1 sequence)

SEQ ID NO: 1 - protein, 6 aa, artificial
  Domain to which a fluorescent dye can attach
  misc_feature (3)..(4): Xaa can be any naturally occurring amino acid
CCXXCC

(19) United States
(12) Patent Application Publication
(10) Pub. No.: US 2018/0238901 A1
(43) Pub. Date: Aug. 23, 2018

(54) HIGH PRECISION SPECTRAL FRET ASSAYS

(71) Applicant: REGENTS OF THE UNIVERSITY OF MINNESOTA, MINNEAPOLIS, MN (US)

(72) Inventors: Tory M. Schaaf, Minneapolis, MN (US); David D. Thomas, Mineapolis, MN (US); Benjamin Grant, Minneapolis, MN (US); Kurt C. Peterson, Saint Paul, MN (US); Gregory D. Gillispie, Falcon Heights, MN (US)

(21) Appl. No.: 15/880,206

(22) Filed: Jan. 25, 2018

Related U.S. Application Data
(60) Provisional application No. 62/450,188, filed on Jan. 25, 2017.

(51) Int. Cl. G01N 33/68; G01N 33/52; G01N 33/542; G01N 33/58

(52) U.S. Cl. CPC G01N 33/6845; G01N 33/52; G01N 2500/20; G01N 33/582; G01N 2500/04; G01N 33/542

(57) ABSTRACT

Provided herein are methods for identifying a compound that alters fluorescence resonance energy transfer (FRET) of a protein. In one embodiment, the method includes providing a target protein, where the target protein includes two heterologous domains, each domain having chromophores that together act as a FRET pair. In another embodiment, the method includes providing a target protein and a second protein, wherein the target protein includes a first heterologous domain including a chromophore, and the second protein includes a second heterologous domain including a chromophore, where the chromophores together act as a FRET pair. The method further includes contacting a sample including the target protein and optional second protein with a test compound to form a mixture, and measuring a fluorescence emission spectrum of the mixture during exposure to a light source. The fluorescence emission spectrum is decomposed into at least two component spectra, where in one embodiment the component spectra include a donor chromophore emission and an acceptor chromophore emission. A ratio (R) is then calculated, and in one embodiment, R is determined according to

$$R = \frac{Acceptor emission}{Donor emission} = \frac{{bF}_{A}}{{aF}_{D}} .$$

### CROSS-REFERENCE TO RELATED APPLICATIONS

This application claims the benefit of U.S. Provisional Application Ser. No. 62/450,188, filed Jan. 25, 2017, which is incorporated by reference herein.

### GOVERNMENT FUNDING

This invention was made with government support under GM027906, HL129814, and DA037622 awarded by National Institutes of Health. The government has certain rights in the invention.

### SEQUENCE LISTING

This application contains a Sequence Listing electronically submitted via EFS-Web to the United States Patent and Trademark Office as an ASCII text file entitled “11005530101SequenceListing_ST25.txt” having a size of 1 kilobyte and created on Mar. 26, 2018. The information contained in the Sequence Listing is incorporated by reference herein.

### SUMMARY OF THE APPLICATION

Provided herein are methods for identifying a compound that alters fluorescence resonance energy transfer (FRET) of a protein. In one embodiment, the method includes (a) providing a target protein, wherein the target protein includes two heterologous domains, wherein a first heterologous domain includes a donor chromophore, and wherein a second heterologous domain includes an acceptor chromophore, wherein the donor chromophore and acceptor chromophore are a FRET pair, and wherein the target protein is cell-associated; (b) contacting a sample including the target protein with a test compound to form a mixture; (c) measuring a fluorescence emission spectrum of the mixture during exposure to a light source, wherein the measuring of the mixture occurs over a period of time no greater than 1 second; (d) decomposing the fluorescence emission spectrum into at least two component spectra, wherein the component spectra include a donor chromophore emission and an acceptor chromophore emission; (e) calculating a ratio (R), wherein the coefficient of variation (CV) of R is no greater than 3%; and (f) identifying whether the test compound present in the sample alters the FRET of the target protein, wherein a difference of at least 1% between the R in the presence of the test compound and the R in the absence of the test compound indicates that the test compound alters the FRET of the target protein.

In one embodiment, the method includes (a) providing a target protein, wherein the target protein includes two heterologous domains, wherein a first heterologous domain includes a donor chromophore, and wherein a second heterologous domain includes an acceptor chromophore, wherein the donor chromophore and acceptor chromophore are a FRET pair, and wherein the target protein is cell-associated; (b) contacting a sample including the target protein with a test compound to form a mixture; (c) measuring a fluorescence emission spectrum of the mixture during exposure to a light source, wherein the measuring of the mixture occurs over a period of time no greater than 1 second; (d) decomposing the fluorescence emission spectrum into component spectra; (e) calculating a ratio (R), wherein R is determined according to

$$R = \frac{Acceptor emission}{Donor emission} = \frac{{bF}_{A}}{{aF}_{D}} ,$$

and wherein the coefficient of variation (CV) of R is no greater than 3%; and (f) identifying whether the test compound present in the sample alters the FRET of the target protein, wherein a difference of at least 1% between the R in the presence of the test compound and the R in the absence of the test compound indicates that the test compound alters the FRET of the target protein.

In one embodiment, the method includes (a) providing a target protein and a second protein, wherein the target protein includes a first heterologous domain including a donor chromophore, wherein the second protein includes a second heterologous domain including an acceptor chromophore, and wherein the donor chromophore and acceptor chromophore are a FRET pair, and wherein the target protein is cell-associated; (b) contacting a sample including the target protein with a test compound to form a mixture; (c) measuring a fluorescence emission spectrum of the mixture during exposure to a light source, wherein the measuring of the mixture occurs over a period of time no greater than 1 second; (d) decomposing the fluorescence emission spectrum into at least two component spectra, wherein the component spectra include a donor chromophore emission and an acceptor chromophore emission; (e) calculating a ratio (R), wherein the coefficient of variation (CV) of R is no greater than 3%; and (f) whether the test compound present in the sample alters the FRET of the target protein, wherein a difference of at least 1% between the R in the presence of the test compound and the R in the absence of the test compound indicates that the test compound alters the FRET of the target protein.

In one embodiment, the method includes (a) providing a target protein and a second protein, wherein the target protein includes a first heterologous domain including a donor chromophore, wherein the second protein includes a second heterologous domain including an acceptor chromophore, and wherein the donor chromophore and acceptor chromophore are a FRET pair, and wherein the target protein is cell-associated; (b) contacting a sample including the target protein with a test compound to form a mixture; (c) measuring a fluorescence emission spectrum of the mixture during exposure to a light source, wherein the measuring of the mixture occurs over a period of time no greater than 1 second; (d) decomposing the fluorescence emission spectrum into component spectra, (e) calculating a ratio (R), wherein R is determined according to

$$R = \frac{Acceptor emission}{Donor emission} = \frac{{bF}_{A}}{{aF}_{D}} ,$$

wherein the coefficient of variation (CV) of R is no greater than 3%; and (f) identifying whether the test compound present in the sample alters the FRET of the target protein, wherein a difference of at least 1% between the R in the presence of the test compound and the R in the absence of the test compound indicates that the test compound alters the FRET of the target protein.

In one embodiment, an altered FRET is the result of a change in structure of the target protein, a change in ligand-binding by the target protein, or a combination thereof. In one embodiment, the target protein and the second protein if present are in a genetically engineered cell, such as a eukaryotic cell, including a vertebrate cell. The target protein and the second protein if present are stably expressed or transiently expressed by the genetically engineered cell. In one embodiment, the target protein and the second protein if present are in a microsomal cellular preparation. In one embodiment, the target protein and the second if are present are in a cell homogenate.

In one embodiment, the donor chromophore is a green fluorescent protein and the acceptor chromophore is a red fluorescent protein, and in another embodiment, the donor chromophore is a cyan fluorescent protein and the acceptor chromophore is a yellow fluorescent protein. In one embodiment, the test compound is an organic molecule. In one embodiment, the measuring of the mixture occurs over a period of time no greater than 0.5 seconds.

In one embodiment, the fluorescence emission spectrum is decomposed into at least four component spectra, wherein the component spectra include a donor chromophore emission and an acceptor chromophore emission, and further include a water Raman emission, and a cell autofluorescence emission. In one embodiment, the fluorescence emission spectrum is decomposed according to

F_{Fit}(λ)=aF_{D}(λ)+bF_{A}(λ)+cF_{C}(λ)+dF_{W}(λ)

In one embodiment, the method further includes determining the FRET efficiency, wherein the FRET efficiency is determined according to

$$FRET = \frac{FR \times QR - AR}{1 + FR \times QR}$$

In one embodiment, the volume of the mixture is no greater than 2 microliters/well. In one embodiment, the method is adapted for use in a high-throughput format. In one embodiment, the measuring occurs over a period of time no greater than 0.5 seconds.

In one embodiment, the method also includes measuring a fluorescence lifetime of the donor chromophore; and calculating the distance distributions and mole fractions of structural states of the target protein, wherein the distance distributions and mole fractions of structural states are calculated according to

$$\begin{matrix}\text{?} & ( Eq . 1 ) \\ \text{?} & ( Eq . 2 ) \\ F ( t ) = \text{?} & ( Eq . 3 ) \\ \text{?} ( t ) = \text{?} ( R ) \text{?} & ( Eq . 4 ) \\ \text{?} & ( Eq . 5 ) \\ \text{?} \\ \text{?} \text{indicates text missing or illegible when filed} & ( Eq . 6 )\end{matrix}$$

Also provided herein is a method for identifying a test compound as a potential false-positive. In one embodiment, the method includes calculating a similarity index (SI), wherein an SI of greater than one standard deviation of the normal distribution of all test compounds indicates a test compound is a fluorescent compound and a potential false-positive. In one embodiment, the SI is determined according to

$$SI = 1 - \text{?}$$

$$\text{?} \text{indicates text missing or illegible when filed}$$

Also provided herein is a computer-implemented method for use in analysis of fluorescence emission data. In one embodiment, the method includes (I) providing a dataset representative of fluorescence emission data obtained for use in analysis of interaction between a target protein and test compounds, wherein providing the dataset includes (a) providing a target protein, wherein the target protein includes two heterologous domains, wherein a first heterologous domain includes a donor chromophore, and wherein a second heterologous domain includes an acceptor chromophore, wherein the donor chromophore and acceptor chromophore are a FRET pair; (b) contacting a plurality of samples including the target protein with test compounds to form a mixture, wherein each sample includes a different test compound; and (c) obtaining a fluorescence emission spectrum of each mixture during exposure to a light source, wherein the dataset includes the fluorescence emission spectrum of each mixture; (II) decomposing each fluorescence emission spectrum of the dataset into at least two component spectra, wherein the component spectra include a donor chromophore emission and an acceptor chromophore emission, wherein the decomposing includes fitting the component spectra to a linear model, determining the contribution of each signal, and using the shape of each component spectra to decompose the fluorescence spectrum; (III) calculating a ratio (R) for each decomposed fluorescence emission spectrum, wherein calculating R includes determining the total fluorescence from the acceptor chromophore and the total fluorescence from the donor chromophore, wherein the coefficient of variation (CV) of R is no greater than 3%; and (IV) identifying whether the test compound present in one of the samples alters the FRET of the target protein, wherein a difference of at least 1% between the R in the presence of the test compound and the R in the absence of the test compound indicates that the test compound alters the FRET of the target protein.

### BRIEF DESCRIPTION OF THE FIGURES

FIG. 1 shows a general block diagram of a general illustrative data processing system for use in analysis of data according to the present disclosure.

FIG. 2 shows a general block diagram of a general illustrative data processing method for analyzing fluorescence emission data according to the present disclosure.

FIG. 3 shows a more detailed block diagram of one illustrative embodiment of a method for providing one or more fluorescence datasets as generally illustrated in the method of FIG. 2.

FIG. 4 shows a more detailed block diagram of one illustrative embodiment of a method for analyzing one or more fluorescence emission datasets as generally illustrated in the method of FIG. 2.

FIG. 5 shows a more detailed block diagram of one illustrative embodiment of a method for identifying one or more mixtures having an R value that differs from a baseline value as generally illustrated in the method of FIG. 2.

FIGS. 6A-D show an overview of spectral unmixing plate reader technology and methods. FIG. 6A. Diagram of the instrument, which provides high-throughput detection of the emission spectrum or lifetime (time-dependent decay). A 473 nm laser (continuous wavelength for spectrum, microchip pulsed for lifetime) excites the sample, and the spectral (fluorescence vs. wavelength) or lifetime (fluorescence vs. time) waveform is recorded directly using a photomultiplier tube (PMT) coupled to a proprietary digitizer or spectrograph, respectively. FIG. 6B. The two-color SERCA (2CS) FRET biosensor, expressed in HEK293 cells, enables measurement of FRET between green (GFP) and red (RFP) fluorescent proteins, positioned at optimized locations on SERCA (Hou, Z., et al., PLoS One, 2012. 7(7):e40369. doi: 10.1371/journal.pone.0040369; Pallikkuth et al., Biophys J, 2013. 105(8): p. 1812-21; Gruber et al., J Biomol Screen, 2014. 19(2): p. 215-22). As depicted, FRET efficiency depends on the structural status of SERCA's domains. FIG. 6C. Reference fluorescence emission (basis) spectra corresponding to one-color SERCA (GFP or RFP), cellular autofluorescence, and water Raman (inelastic light scattering), normalized to GFP. These basis spectra were used to analyze the spectra of cells expressing 2CS (FIG. 6D), to decompose the observed spectrum of 2CS into a linear combination of component spectra (best fit shown in dashed black, components shown with same labeling scheme as in FIG. 6C), thus permitting accurate quantitation of the GFP (donor) and RFP (acceptor) fluorescence needed to calculate FRET (13.7% for data shown in FIG. 6D, with SD=0.11%), using (Eq. 4). Complete emission spectra from 500-700 nm were acquired in less than 3 minutes for a full 384-well plate.

FIGS. 7A-D show analysis of mixtures of GFP- and RFP-expressing stable cell lines. FIG. 7A. Cells expressing a GFP-only control construct were mixed with cells expressing a RFP-only control construct in a 384-well plate. The % of GFP cells was varied as indicated, but the total volume and number of cells remained constant. The observed spectrum of each GFP mixture is shown (average from 32 wells). Each observed spectrum was fit using linear least-squares minimization to determine the four scalar coefficients (Eq. 3), and the results are plotted in FIG. 7B, showing that the fluorescence contributions of GFP and RFP to the emission spectrum vary inversely and linearly. FIG. 7C shows the results of A after subtracting contributions from autofluorescence and Raman. FIG. 7D shows the residuals from fits in B, offset vertically for clarity.

FIGS. 8A-D show spectral and lifetime mode comparison of 2CS concentration-dependent FRET change in response to the known SERCA inhibitor thapsigargin. FIG. 8A. Fluorescence emission spectra (average of 32 wells) normalized to GFP intensity, showing 8% reduction in FRET (decreased RFP emission) in response to 50 nM thapsigargin. FIG. 8B. Nanosecond time-resolved fluorescence decay waveforms (average of 32 wells) normalized to GFP-only, showing a 6% reduction in FRET (longer GFP lifetime) in response to 50 nm thapsigargin. FIG. 8C. Heat map of FRET efficiency calculated from spectral mode. 10 different concentrations of thapsigargin (0.8, 1, 2, 3, 4, 5, 6, 8, 10, and 50 nM, increasing left to right) were dispensed across a 384-well plate, with DMSO controls at columns 1, 2, 23, and 24. FIG. 8D. Heat map of FRET efficiency calculated from lifetime mode from the same plate.

FIGS. 9A-D show concentration-response curves and precision of known SERCA effectors. FIG. 9A and FIG. 9 each show the concentration-dependent 2CS FRET response of three well-established SERCA inhibitors, thapsigargin (Tg), 4-dihydroxy-2, 5-di-tert-butylbenzene (BHQ), and cylcopiazonic acid (CPA), in a 384-well plate (n=8 wells for each concentration). Fits to the Hill equation (curves) give EC50 values in close agreement for the two modes, and in good agreement with published data on SERCA inhibition. Histograms illustrated in FIG. 9C (Spectral Mode) and FIG. 9D (Lifetime Mode) illustrate the precision of FRET determination from 384 wells of 2CS cells without the addition of drug. Mean and standard deviation values for FRET, determined from the Gaussian fits shown in the solid black trace, were 13.7%±0.11 for spectral and 13.6%±0.36 for lifetime.

FIGS. 10A-D show spectral fitting increases assay precision by solving for the contribution of cellular autofluorescence. FIG. 10A. Spectra were obtained from mixtures of transfected cells (expressing 2CS), with the indicated % of untransfected cells. Each spectrum is the average from 16 wells. FIG. 10B. Example of data analysis using Eq. 3, showing the fit to the data in A for the case of 80% untransfected cells. FIG. 10C. Autofluorescence (c in Eq. 3, normalized to the sum of all four components) from fits. FIG. 10D. Quality factor Z¢ (Cornea et al., J Biomol Screen, 2013. 18(1): p. 97-107; Zhang et al., J Biomol Screen, 1999. 4(2): p. 67-73), using the effect of 100 nM Tg (FIG. 4) to define the signal window.

FIGS. 11A-D show the accurate FRET efficiency determination from CFP and YFP biosensors in HEK293 cells. FIG. 11A. Two-component spectral fit of the C17V FRET standard. FIG. 11B. FRET data from three CFP-YFP FRET pairs with different lengths. 48 wells for each of the three pairs were studied in a 384-well plate. FIG. 11C. Concentration-response curves showing the effects of three SERCA inhibitors on FRET (120 min after mixing), using the CFP/YFP-based D1ER cameleon FRET calcium sensor (n=8 wells for each concentration). Curves show best fits to the Hill equation. FIG. 11D. Time-dependent effects of SERCA inhibitors on ER Calcium at saturating drug concentrations. DMSO controls have no effect on ER calcium concentrations (data not shown). Thapsigargin (Tg) irreversibly binds SERCA with high-affinity and depletes calcium at a faster rate than BHQ and CPA, which have micromolar binding affinities.

FIGS. 12A-C show SUPR water raman test comparison to commercially available spectral readers. FIG. 12A. Water Raman spectra acquired on commercially-available fluorescence microplate readers. Total acquisition time is approximately 2 minutes for each spectrum (one second per wavelength). FIG. 12B. Water Raman spectrum acquired using a cuvette-based spectrometer with flash lamp excitation. FIG. 12C. Water Raman spectra acquired from six separate wells of one 384 well plate using the spectral unmixing plate reader. The six raw spectra are overlaid to demonstrate the reproducibility across measurements.

FIG. 13 shows the determination of autofluorescence reference spectrum.

FIG. 14 shows the determination of GFP reference spectra.

FIG. 15 shows HEK293 cells overexpressing an acceptor-only control construct. TagRFP was fused to SERCA2a used to generate the tagRFP reference spectrum. A longer wavelength excitation (532 nm) was used, so the subtraction of cellular autofluorescence and the water Raman signal are not required.

FIG. 16 shows the fluorescence lifetime of cells overexpressing the 2CS biosensor was used to evaluate the FRET response to the known inhibitor thapsigargin as previously shown in FIG. 9B. The FRET efficiency at each dose of thapsigargin is plotted on the y-axis where FRET decreases at higher concentrations of thapsigargin. The FRET values obtained from lifetime mode using Eq 2 were used to solve for the β-factor (CCD detector sensitivity across wavelengths). The β-factor was then used to determine the FRET efficiency (Eqs 4) across all 12 doses of the thapsigargin concentration curve (shown on the y-axis).

FIGS. 17A-B show the fluorescence lifetime of the donor-only (GFP) one-color SERCA control. Evaluated using a single-exponential fit, as shown in FIG. 17A, and a two-exponential fit, as shown in FIG. 17B. For each fit, the raw donor-only waveform is shown in black and the instrument response function with a dotted line. The residuals from each fit across the nanosecond time domain (x-axis) is shown below the fluorescence decay waveforms and used to determine the χ2. As shown the χ2 was reduced for the two-exponential fit and the average lifetime was found to be 2.58 ns.

FIGS. 18A-B shows an overview of high-throughput spectral and lifetime FRET drug screening. Conceptual data shown in FIG. 18A (lifetime mode) and FIG. 18B (spectral mode) illustrate the dependence of fluorescence signals on FRET. Solid black curve (D): donor only (no FRET). Dashed black curve (DA): donor plus acceptor (FRET). In FIG. 18B, dotted curves show the resolution of the spectrum into components corresponding to donor (GFP) and acceptor (RFP) emission.

FIGS. 19A-D show lifetime analysis of time-resolved fluorescence decay waveforms resolves structural states of the 2CS biosensor. FIG. 19A. Two structural states of SERCA are resolved, corresponding to two Gaussian interprobe distance distributions, consistent with a two-state structural model with an equilibrium between closed (5.5 nm FRET distance) and open (10.2 nm FRET distance) structural states. FIG. 19B. The addition of a saturating dose (50 nM) of the known inhibitor thapsigargin (Tg) shifts this equilibrium substantially toward the open state. FIG. 19C. Concentration dependence of Tg effect on the structural distribution (n=8 wells for each concentration). FIG. 19D. Plot, based on FIG. 19C, of closed state mole fraction vs Tg.

FIGS. 20A-D show an example of spectral and lifetime pilot drug screening. FIG. 20A. Fluorescence emission spectra were used to identify and flag potential interference from fluorescent compounds by assessing the similarity index (Eq. 15) of each well from a pilot screen of the NCC1 & 2 small-molecule libraries. A control spectrum (% v/v DMSO well) and non-fluorescent (compound not identified as a FRET hit during screening with 2CS) have a high degree of similarity, as shown as direct overlap of the spectra of the control and the non-fluorescent compound. A slightly fluorescent compound is also depicted and was flagged as a potential false-positive hit. The fluorescent profile of all 1152 wells from one NCC screen was assessed using a stringent similarity index threshold; 44 compounds were flagged as potential false-positives due to interference from compound fluorescence. FIG. 20B. Histogram plots of the wells from one NCC screen after removing potential fluorescent compounds. Gaussian fits depict an increase in precision from spectral mode (left) in comparison to lifetime mode (right), shown as the frequency of FRET efficiency determined by either method and a narrower distribution from spectral mode (average FRET calculated by spectral unmixing or lifetime and the standard deviation determined from the Gaussian fit). FIG. 20C. One 2CS pilot NCC screen (spectral mode) is shown with a hit threshold set at a 0.02 change in 2CS FRET (4 SD). 16 FRET hits were identified in this screen. 11 of these 2CS FRET hits were found to be reproducible across three independent screens (shown as triangles). FIG. 20D. The same 384-well plates were scanned in lifetime mode. 16 hits were identified using the same threshold set at a 0.02 change in 2CS FRET efficiency (3 SD). In this screen, nine of the reproducible 2CS FRET hits identified in spectral mode were also FRET hits as assessed by lifetime mode (shown as triangles).

FIGS. 21A-D show reproducible FRET hits assessed across independent screens and time course studies. FIG. 21A. Spectral mode identified eleven reproducible 2CS FRET hits using a threshold of 0.02 change in FRET (horizontal line). The reproducibility of each 2CS FRET hit, after 20 min incubation, across three independent screens is shown. The ΔFRET from each compound remains consistent from screen to screen. ΔFRET was calculated by assessing the change in FRET of 2CS from the average FRET, determined by the Gaussian fit of all wells not flagged as fluorescence compounds. FIG. 21B. Lifetime mode assessment of the eleven reproducible spectral FRET hits. Nine of the eleven FRET hits were reproducible (triplicate) hits using a 0.02 FRET threshold. Compounds #106 and 190 were not identified as lifetime FRET hits in one of the three independent screens. FIG. 21C. Spectral FRET hits evaluated by time-course studies. Each independent pilot screen was scanned in spectral mode after 20, 60, 90, and 120 minutes of compound incubation. The change in 2CS FRET efficiency (ΔFRET) of each compound is plotted and each reproducible FRET hit, identified as a hit using the spectral unmixing method, remained a hit over multiple time points. The 2CS FRET hits depicted here were from screen 2 (see Screen 2 in FIGS. 21A and 21B). Compounds #60, 94, 459, 639, and 660 exhibited an increased FRET change over time. FIG. 21D. Lifetime FRET change evaluated by time-course studies. Ten compounds from screen 2 were identified as hits, after 20 minutes of compound incubation, using a threshold set at 0.02 FRET change (horizontal line). Compounds #32 and 356 displayed a modest reduction in 2CS ΔFRET at the later time points. Compounds #60, 94, 459, 639, and 660 again exhibited an increased FRET change over time.

FIGS. 22A-D show multi-parameter concentration-dependent effect of FRET hits. FIG. 22A. Spectral mode analysis of the reproducible 2CS FRET hits. Compounds were dispensed into 384 well plates, across an eight-point concentration-gradient (n=4 for each concentration). Six representative compounds produced a dose-dependent FRET change as evaluated in spectral mode. These compounds altered FRET with micromolar affinities with subtle differences across the compounds. FIG. 22B. The same 384-well plate was evaluated using lifetime mode and demonstrated excellent agreement in the dose-dependent FRET change across two independent FRET measurements. FIG. 22C. Global analysis of the lifetime data depicts a dose-dependent change in the mole fraction of the closed 2CS headpiece (5.5 nm distance distribution). Using this distance distribution model, the confirmed reproducible hits perturbed the 2CS structural equilibrium between open and closed states. All of the hits decreased 2CS FRET, indicating increased distance between GFP and RFP. FIG. 22D. Water Raman spectrum acquired from compound-only wells of the known compound aggregator miconazole demonstrates ultra-high-sensitivity of spectral recording. Compound aggregation dose-dependently causes more light to be absorbed and decreases inelastic light scattering (Raman band).

FIGS. 23A-D show functional characterization of FRET hits on SERCA ATPase activity and ER calcium content. FIG. 23A. 2CS FRET hits inhibit SERCA ATPase activity. NADH-enzyme coupled activity assay of purified SERCA was measured at eight different concentrations of the reproducible FRET hits. The maximal rate of SERCA activity was measured at saturating calcium (10 μM) after 20 minute incubation with compounds and dose-dependent inhibition was observed. FIG. 23B. Endoplasmic reticulum calcium was depleted by the 2CS FRET hits. ER calcium was monitored in live-cells overexpressing the endoplasmic reticulum localized calcium FRET sensor (D1ER). D1ER FRET is dependent on calcium concentration, where less calcium causes a reduction in FRET. ER calcium levels were monitored over time in response to an eight-point concentration gradient of each hit compound. A 384 well plate was repeatedly scanned (every three minutes) with D1ER cells. The dose-dependent FRET change (ER calcium depletion) after 120 minutes compound incubation is shown and depicts differential depletion at each compound concentration. FIG. 23C. Maximal ER calcium depletion in the presence of a saturating dose (50 μM) of each compound (decreased D1ER FRET) was assessed over a 120-minute period. The 2CS FRET hits displayed time-dependent and compound-specific ER calcium depletion. Miconazole exhibited both maximal SERCA ATPase Vmax inhibition and the largest amount of ER depletion. FIG. 23D. Structure and activity assay correlation of 2CS FRET hits. The maximal change (percent change) of the structural FRET change from the 2CS FRET biosensor as well as the maximal change from two different functional assay (ATPase activity assay and D1ER calcium depletion) are shown.

### DETAILED DESCRIPTION OF ILLUSTRATIVE EMBODIMENTS

The description that follows is not intended to describe each disclosed embodiment or every implementation of the present disclosure. In several places throughout the application, guidance is provided through lists of examples, which examples can be used in various combinations. In each instance, the recited list serves only as a representative group and should not be interpreted as an exclusive list.

Provided herein are methods for identifying a compound that alters fluorescence resonance energy transfer (FRET) of a target protein. The terms target protein and biosensor are used interchangeably. The disclosure is not limited by the target protein that is used in the methods. Examples of suitable target proteins include, but are not limited to, cytoplasmic proteins, membrane-bound proteins, proteins that include one or more transmembrane regions, and nuclear proteins. A target protein used in the methods may be a subunit of a multimeric protein, where the protein is a homomeric or a heteromeric protein. A target protein may be globular or fibrous. In one embodiment, a target protein is a channel or a pump. In one embodiment, a target protein is associated with the sarcoplasmic reticulum of a cell. Specific examples of target proteins include, but are not limited to, sarco(endo)plasmic reticulum calcium adenosine triphosphatase (SERCA) (Robia, US Patent Application 2013/0231262; Hou et al., 2012, PLoS One, 7:e40369), sodium/potassium ATPase (NKA) (Song et al., 2011, J Biol Chem 286:9120-9126), an ABC transporter MRP1 (Marquez et al., 2011, Curr Drug Targets 12:600-620), a ryanodine receptor (U.S. patent application Ser. No. 14/565,811, Cornea et al.), myosin (Muretta et al., 2013, Proc Natl Acad Sci USA 110:7211-7216), calmodulin kinase II (Erickson et al, 2013, Nature 502:372-376), protein kinase D (Chang et al., 2011, J Biol Chem. 286:33390-33400), ataxin-1 (Lagalwar and Orr, 2013, Methods Mol Biol 1010:201-209), death receptor 5 (Valley et al., 2012, J Biol Chem 287:21265-21278), the D1ER calcium FRET sensor (Palmer et al., Proc Natl Acad Sci USA 2004, 101, 17404-17409; Palmer et al., Nat Protoc 2006, 1, 1057-1065), or intermolecular interactions such as SERCA/PLB (Gruber et al., 2012, Biochem Biophys Res Commun 420:236-240).

Chromophores suitable for the methods described herein are known to the skilled person and are routinely used. Examples of suitable chromophores include, but are not limited to, fluorescent proteins, including green fluorescent protein, red fluorescent protein, yellow fluorescent protein, blue (cyan) fluorescent protein, and orange fluorescent protein. Green fluorescent protein and red fluorescent protein may be used as a donor-acceptor pair, and blue fluorescent protein and yellow fluorescent protein may be used as a donor-acceptor pair. The amino acid sequences of different versions of green fluorescent protein, red fluorescent protein, yellow fluorescent protein, and blue fluorescent protein are known to the skilled person and are readily available, as are analogues of these proteins. Other chromophores include fluorescent dyes, such as fluorescent dyes that can be attached to a protein, such as when the protein is present in a cell or purified from a cell. Examples of such dyes are known in the art and are routinely used. Examples include dyes that react with cysteine, having reactive iodoacetamide, maleimide, or thiosulfonate groups. Other examples include the protein labeling reagent FLASH-EDT2, a dye that can react with the domain CCXXCC (SEQ ID NO:1) (Invitrogen), and SNAP-tag, a self-labeling protein tag (New England Biolabs). Other fluorescent dyes are available that react specifically with an unnatural amino acid that is incorporated into a target protein by a modified tRNA. In one embodiment, a fluorescent dye is one that will pass through a cell membrane.

Any appropriately selected two chromophores can be used as a donor-acceptor pair in the methods described herein, provided that the energy emitted by a donor (the emission spectrum) overlaps with the energy absorbed by an acceptor (the excitation spectrum), e.g., a fluorescence resonance energy transfer process (FRET) occurs between two chromophores. In one embodiment, donor-acceptor pairs are chosen such that interference from cell autofluorescence or test-compound fluorescence is minimized. Accordingly, in one embodiment, donors that can be excited at longer wavelengths are superior to those excitable at shorter wavelengths. Also, probes with longer (more than 3 nanoseconds (ns)) fluorescence lifetimes (FLT) may be superior to probes with shorter FLT.

A target protein used in the methods described herein includes at least one heterologous domain. In one embodiment, a target includes one or two heterologous domains. As used herein, a “heterologous domain” refers to a foreign sequence, e.g., one or more amino acid (e.g., a fluorescent protein, or a domain to which a fluorescent dye can attach) that is not normally part of a wild-type protein. Examples of domains to which a fluorescent dye can attach include an amino acid sequence, such as CCXXCC (SEQ ID NO:1), or an unnatural amino acid.

In one embodiment, a target protein includes two heterologous domains, where one of the heterologous domains is a donor probe, and the second is an acceptor probe. In one embodiment, each heterologous domain is a fluorescent protein. In one embodiment, one heterologous domain is a fluorescent protein and the other is a domain to which a fluorescent dye can attach. In one embodiment, each heterologous domain is a domain to which a fluorescent dye can attach. This type of target protein permits analysis of intramolecular FRET.

In one embodiment, a target protein includes one heterologous domain. The heterologous domain may be a donor probe or an acceptor probe. The heterologous domain may be a fluorescent protein or may be a domain to which a fluorescent dye can attach. In one embodiment, the method of using the target protein having one heterologous domain includes fluorescence lifetime analysis of the probe that does not include FRET. In such an embodiment, the probe is typically a fluorescent dye that is attached to the target protein, and a change in fluorescence is suggestive of a change in structure.

In one embodiment, the method of using the target protein having one heterologous domain includes the use of a second protein. The second protein is a protein that binds to the target protein. An example of such protein pairs include SERCA and PLB, and RyR and calmodulin, and many other proteins pairs are known to the skilled person. The second protein includes the second heterologous domain. This type of target protein permits analysis of intermolecular FRET.

In those embodiments where the target protein includes two heterologous domains, each heterologous domain may be present at any location in a target protein. In those embodiments where the target protein includes one heterologous domain and a second protein includes the second heterologous domain, the heterologous domain, whether it is in the target protein or the second protein, may be present at any location in either protein. Thus, a heterologous domain may be at the amino-terminus of a protein, the carboxy-terminus of a protein, or at a location within the protein. However, while the two heterologous domains can be independently located, the two heterologous domains are present at two locations that are close enough to allow FRET to occur between the two. Thus, in one embodiment, a target protein includes a donor probe domain and an acceptor probe domain, where the distance between them in the tertiary structure of the target protein is estimated to be no greater than 2 nanometers (nm), no greater than 4 nm, no greater than 6 nm, no greater than 8 nm, no greater than 10 nm, or no greater than 12 nm. In one embodiment, a target protein includes two heterologous domains, where the distance between them in the primary structure of the target protein is at least 3 amino acids, at least 5 amino acids, at least 10 amino acids, at least 50 amino acids, or at least 100 amino acids. Likewise, in one embodiment, a target protein includes a heterologous domain and a second protein includes a second heterologous domain, where the distance between them in the tertiary structure when the two proteins are bound is estimated to be no greater than 2 nanometers (nm), no greater than 4 nm, no greater than 6 nm, no greater than 8 nm, no greater than 10 nm, or no greater than 12 nm.

In one embodiment, a target protein used in the methods has at least one function in a cell. The role may be related to the mechanical, physical, and/or biochemical function of a cell. For instance, the target protein may bind to another molecule (e.g., an interaction between two subunits of a multimeric protein, an interaction between a target protein and another protein, or an interaction between a target protein and a non-protein ligand), have an enzymatic activity, or a combination thereof. The addition of the two probe domains may alter the function of the target polypeptide in some way. Thus, in one embodiment, the function of a target protein that includes the two probe domains does not have any detectable change. In another embodiment, the function of a target protein that includes the two probe domains does have a detectable change. Target proteins useful in the methods described herein may have altered function, but preserve one or more essential characteristics that can be analyzed as disclosed herein. In some embodiments, a target protein is a wild-type (e.g., it is a wild-type protein modified to include the two heterologous probe domains), and in other embodiments the target protein can include one or more mutations associated with altered function of that target protein.

Methods for engineering proteins to include one or more heterologous domains are known in the art and are routine. Typical locations for an inserted heterologous domain include the N-terminus, the C-terminus, and an internal site. Suitable internal sites can be predicted by analysis of a crystal structure of a protein and identification of loop (e.g., not a recognizable helix or sheet) that is exposed to the surface of the protein.

A target protein can be expressed in a cell. A polynucleotide sequence encoding the target protein with the two probe domains can be readily produced by reference to the standard genetic code using known and routine methods, and the polynucleotide can be inserted into a vector. A vector is a replicating polynucleotide, such as a plasmid, phage, or cosmid, to which another polynucleotide may be attached so as to bring about the replication of the attached polynucleotide. Construction of vectors containing a polynucleotide encoding a target protein employs standard ligation techniques known in the art. See, e.g., Sambrook et al, Molecular Cloning: A Laboratory Manual, Cold Spring Harbor Laboratory Press (1989) or Ausubel, R. M., ed. Current Protocols in Molecular Biology (1994). A vector can provide for further cloning (amplification of the polynucleotide), i.e., a cloning vector, or for expression of the polypeptide encoded by the coding region, i.e., an expression vector. The term vector includes, but is not limited to, plasmid vectors, viral vectors, cosmid vectors, or artificial chromosome vectors.

The cell may be a prokaryotic cell or a eukaryotic cell. Examples of eukaryotic cells include mammalian cells, such as vertebrate cells, e.g., human, murine (including mouse and rat), canine, or porcine cells. Other examples of eukaryotic cells include invertebrates (such as parasites, including helminths and protozoans such as Plasmodium spp.) and unicellular eukaryotic cells, such as yeast cells. Examples of prokaryotic cells include, for instance, E. coli. The types of cells useful for expression and analysis of a target protein will vary depending on the target protein, and skilled person will be able to determine which cells can be used based on prior reports of expression of a target protein. For instance, when the target protein is one expressed in muscle cells, such as RyR or SERCA, suitable cells include myocytes, for example ventricular myocytes and cardiomyocytes. In some embodiments, a dysfunctional cell may be used. For example, the cell may be a cancer cell, or myocytes from muscle that is dysfunctional, e.g., failing heart, pathologically stressed, or dystrophic, may be used. In one embodiment, the cell is a cell that can be cultured in suspension (e.g., non-adherent) and does not require contact with a surface for replication. In one embodiment, expression of the target protein in a cell is stable, e.g., an exogenous polynucleotide encoding the target protein is integrated into the genomic DNA of a cell. In one embodiment, the expression of the target protein in a cell is transient.

The methods provided herein include measuring the fluorescence emission spectrum (e.g., the spectral domain), the fluorescence lifetime, or both.

A measuring instrument useful in the methods described herein is a spectrometer that is compatible with FRET assays. An example of a measuring instrument useful for obtaining fluorescence emission spectra is described in Examples 1 and 2. An example of a measuring instrument useful in obtaining fluorescence lifetime data is described in Thomas et al. (US Published Patent Application 2015/0204847). A single instrument can be used to obtain both spectral and lifetime data, or separate instruments can be used.

The combination of both spectral and lifetime fluorescence recording methods creates a novel screening platform offering the advantages of both methods. Lifetime-based screening offers high-precision measurements that are not dependent on the intensity of the signal. Lifetime data can resolve the amount of different fluorescence species emitted from the same fluorophore. Spectral-based screening offers increased precision due to the resolution across the fluorescence spectrum. Spectral data can resolve the amount of different fluorescence species emitted across the full emission spectrum including inelastic light scatter from the water Raman signal. This information can be used to determine the amount of material in each well, background signal from fluorescence sources such as cellular autofluorescence, or artifacts from fluorescent small-molecules. When these methods are combined for high throughput screening, it creates a novel platform where incredibly precise and fast measurements from the emission spectrum and the nanosecond decay rate can be coupled and compared.

An advantage of spectral mode in comparison to lifetime mode is that spectral unmixing methods removes cellular autofluorescence and water Raman contributions, so assay quality (Z′ and % CV) is high, even when cell protein expression is very low. High-precision spectral measurements can be acquired from a minute amount of material and/or signal.

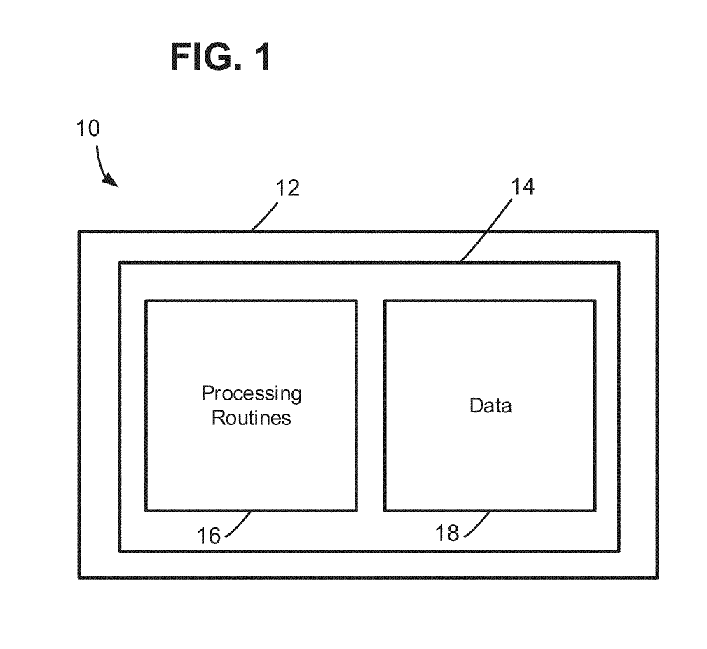

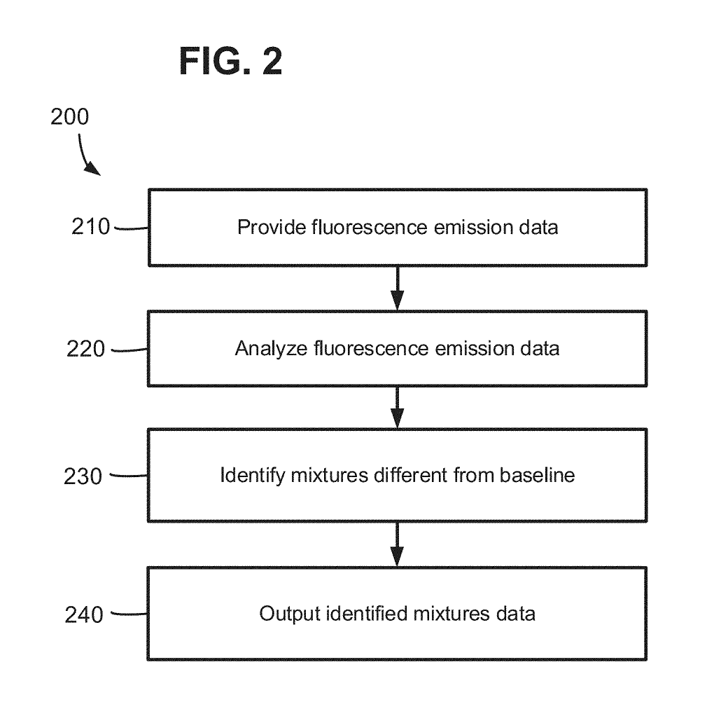

FIG. 1 shows a data analysis system 10 including a processing apparatus (block 12) and data storage (block 14). Data storage (block 14) allows for access to processing programs or routines (block 16) and one or more other types of data (block 18) that may be employed to carry out the illustrative fluorescence emission data analysis method (block 200) as shown generally in the block diagram of FIG. 2.

For example, processing programs or routines (block 16) may include programs or routines for performing matrix mathematics, compression algorithms, standardization algorithms, comparison algorithms, vector mathematics, or any other processing required to implement one or more embodiments of the present disclosure as described herein. Data (block 18) may include, for example, wavelength data representative of fluorescence emission data taken at a single time point or taken over time, data representative of decomposed fluorescence spectra taken at a single time point or taken over time, data representative of ratios for each decomposed fluorescence spectrum taken at a single time point or taken over time, data representative of FRET efficiency data taken at a single time point or taken over time, results from one or more processing programs or routines employed according to the present disclosure, or any other data that may be necessary for carrying out the one or more processes described herein.

As used herein, fluorescence emission data refers to any data generated by exposing a mixture of a cell-associated target protein to a light source. Examples of light sources include, but are not limited to, LED light sources and continuous wave lasers with 405 (CFP), 440 (CFP), 488 (GFP), 532 (OFP), or 560 nm (RFP) can be used to excite the donor chromophores. Laser-driven light sources, white light sources with excitation filters, or monochromators can also be used to excite donor chromophores across the visible spectrum (for instance, 434 nm excitation for a D1ER target protein) and adjusted to the peak excitation of the donor chromophore). In one embodiment, the use of two or more fluorescence excitation wavelengths can be used to generate multiplexed datasets, e.g., spectra emission spectrum from two or more excitation wavelengths, or fluorescence lifetimes from two or more excitation wavelengths. These multiplexed data sets could contain emission spectra or fluorescence emission decays (lifetime) obtained from donor chromophore excitation, as described herein, as well as, those obtained from acceptor excitation.

In one or more embodiments of the present disclosure, the data analysis system 10 may be implemented using one or more computer programs executed on programmable computers, such as computers that include, for example, processing capabilities, data storage (e.g., volatile or non-volatile memory and/or storage elements), input devices, and output devices. Program code and/or logic described herein may be applied to input data to perform functionality described herein and generate desired output information. The output information may be applied as input to one or more other devices and/or processes as described herein or as would be applied in a known fashion.

The program used to implement the present disclosure may be provided using any programmable language, e.g., a high level procedural and/or object orientated programming language that is suitable for communicating with a computer system. Any such programs may, for example, be stored on any suitable device, e.g., a storage media, readable by a general or special purpose program, computer or a processor apparatus for configuring and operating the computer when the suitable device is read for performing the procedures described herein. In other words, at least in one embodiment, the system 10 may be implemented using a computer readable storage medium, configured with a computer program, where the storage medium so configured causes the computer to operate in a specific and predefined manner to perform functions described herein.

Likewise, the data analysis system 10 may be configured at a remote site (e.g., an application server) that allows access by one or more users via a remote computer apparatus (e.g., via a web browser), and allows a user to employ the functionality according to the present disclosure (e.g., user accesses a graphical user interface associated with the program to analyze fluorescence emission data).

The processing apparatus (block 12), may be, for example, any fixed or mobile computer system (e.g., a personal computer or mini computer). The exact configuration of the computing apparatus is not limiting and essentially any device capable of providing suitable computing capabilities may be used according to the present disclosure. Further, various peripheral devices, such as a computer display, mouse, keyboard, memory, printer, scanner, spectrometer, any imaging apparatus capable of simultaneously or quasi-simultaneously acquiring images at more than one wavelength, etc., are contemplated to be used in combination with processing apparatus (block 12) of the data analysis system (block 14).

In view of the above, it will be readily apparent that the functionality as described in one or more embodiments according to the present disclosure may be implemented in any manner as would be known to one skilled in the art. As such, the computer language, the computer system, or any other software/hardware which is to be used to implement the present disclosure shall not be limiting on the scope of the processes or programs (e.g., the functionality provided by such processes or programs) described herein.

One will recognize that a graphical user interface may be used in conjunction with the embodiments described herein. The user interface may provide various features allowing for user input thereto, change of input, importation or exportation of files, or any other features that may be generally suitable for use with the processes described herein. For example, the user interface may allow default values to be used or may require entry of certain values, limits, threshold values, or other pertinent information.

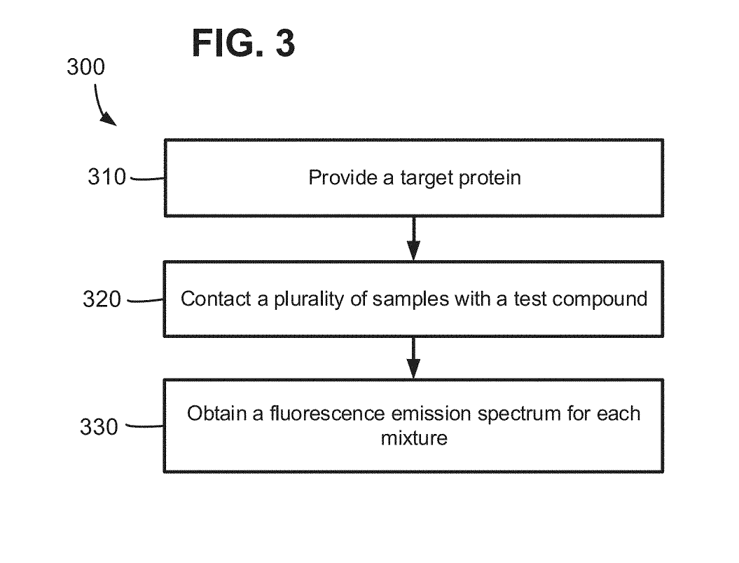

FIG. 2 shows a general block diagram of an illustrative data processing method 200 for analyzing fluorescence emission data according to the present disclosure. One will recognize that one or more of the blocks of functionality described therein may be carried out using one or more programs or routines. The method 200 may be implemented in one or more various manners, some examples of which will be further described herein with reference to FIGS. 3-5.

Generally, the processing method 200 includes providing fluorescence emission data (block 210) (e.g., a fluorescence emission dataset representative of fluorescence emission spectrum data for individual mixtures obtained at a single time point, etc.). Typically, one or the average of more than one of the individual mixtures that do not contain a test compound is used as a baseline or control. The fluorescence emission dataset is analyzed (block 220). The analyzed fluorescence emission dataset is then used to identify individual mixtures that are different from the baseline (block 230). In one or more embodiments, such data of identified individual mixtures can then be outputted (block 240) to at least one of, e.g., a user, a display, a printer, and/or a file. Further, the output may be analyzed by a user, used by another machine that provides output based thereon, etc.

In one embodiment, providing fluorescence emission data (block 210) includes use of a multi-well plate, where each well contains an individual mixture. As described in greater detail herein, a multi-well plate includes at least one baseline well that is used to obtain a baseline value, and other wells that are identical to the baseline well but further include a test compound. In such an embodiment, each well of the multi-well plate can be an individual value of a fluorescence emission dataset, or several wells can be combined to provide an individual value of a fluorescence emission dataset. An example of a 384-well plate is shown in Example 1, FIGS. 3C and 3D. The wells of columns 1, 2, 23, and 24 are baseline wells, e.g., they do not contain a test compound, and are averaged to obtain a baseline value. The remaining wells contain test compounds.

As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by processing apparatus (block 14) described herein.

As described herein, a file in user-readable format may be any representation of data (e.g., ASCII text, binary numbers, hexadecimal numbers, decimal numbers, audio, graphical) presentable on any medium (e.g., paper, a display, sound waves, etc.) readable and/or understandable by a user.

Generally, the methods according to the present disclosure, as will be further described herein, may use algorithms implementing mathematics to transform the fluorescence emission data into analyzed fluorescence emission data.

One or more fluorescence emission datasets are provided (block 210) as part of method 200. One or more embodiments of providing a fluorescence emission dataset are further described in detail with reference to FIG. 3. For example, providing one or more fluorescence emission datasets may include multiple steps such as, but not limited to, providing a target protein (e.g., block 310), contacting a plurality of samples containing the target protein with a test compound to result in a mixture (e.g., block 320), and obtaining a fluorescence emission spectrum for each mixture (e.g., block 330), where the fluorescence emission spectrum for each mixture is the fluorescence emission dataset. Optionally and preferably, the fluorescence emission dataset includes a baseline value.

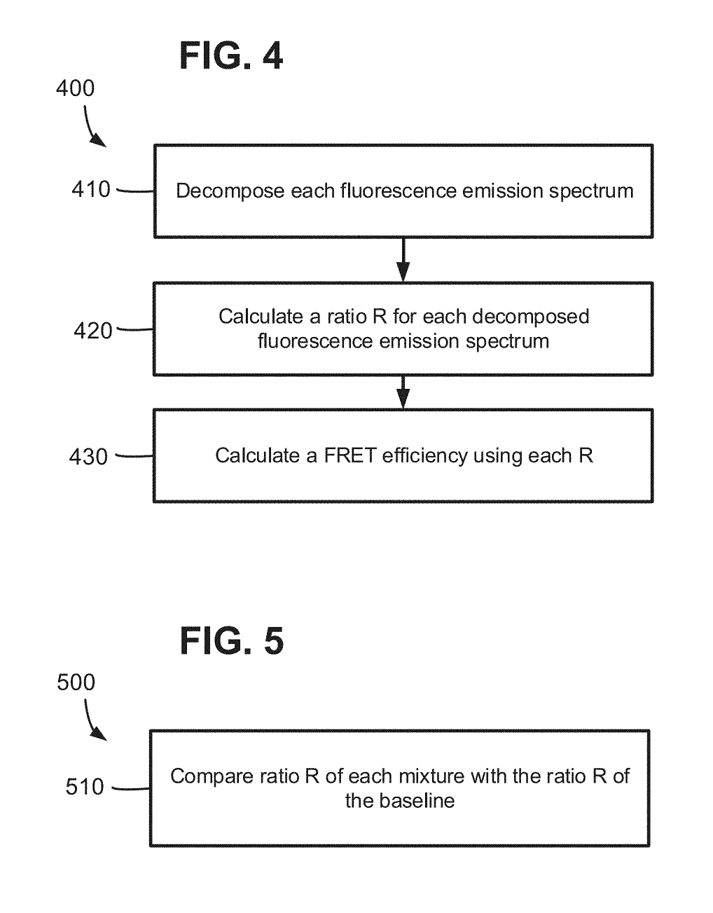

One or more fluorescence emission datasets are analyzed (block 220) as part of method 200. One or more embodiments of analyzing a fluorescence emission dataset are further described in detail with reference to FIG. 4. For example, analyzing a fluorescence emission dataset may include multiple steps such as, but not limited to, decomposing each fluorescence emission spectrum (e.g., block 410), and calculating a ratio R for each decomposed emission spectrum (e.g., block 420). Optionally, FRET efficiency values for each R can be calculated (e.g., block 430).

One or more mixtures different from the baseline are identified from one or more analyzed fluorescence emission datasets (block 230) as part of method 200. One or more embodiments of identifying one or more mixtures different from the baseline are further described in detail with reference to FIG. 5. For example, identifying one or more mixtures that differ from the baseline may include multiple steps such as, but not limited to, comparing the R value for each mixture with the R value of the baseline (e.g., block 510).

Further details of various embodiments are described below. While the various embodiments are described for an individual mixture, the skilled person will recognize that such a method can be carried out by testing a plurality of mixtures as described, for instance, at FIG. 2-5.

In one embodiment, a method includes identifying a compound that alters FRET of a target protein described herein (e.g., block 230). In one embodiment, the method includes providing a target protein (e.g., block 310) with two heterologous domains, where one domain is a donor chromophore and the second domain is an acceptor chromophore, and the donor chromophore and acceptor chromophore are a FRET pair. In another embodiment, the method includes providing a target protein (e.g., block 310) with one heterologous domain and a second protein that includes a second heterologous domain, where one of the domains is a donor chromophore and the other domain is an acceptor chromophore, and the two chromophores are a FRET pair. Thus, if the target protein includes the acceptor domain, then the second protein includes the donor domain. In one embodiment, a chromophore is a fluorescent protein, and in another embodiment a chromophore is a fluorescent dye. In those embodiments where the chromophore is a fluorescent dye, the heterologous domain of the protein (i.e., the target protein or the second protein), is a domain to which a fluorescent dye can attach.

The target protein can be cell-associated. A cell-associated protein is one that is present as part of an intact live cell, a permeabilized cell, or an extraction of a live cell. In one embodiment, the target protein can be a purified protein. An extraction of a live cell includes, but is not limited to, a homogenized cell extract or an enrichment of a subcellular fraction, such as a microsomal preparation. The term “homogenized” refers to any method of disrupting cells, including use of detergent, a homogenizer, French press, etc. In one embodiment, the cell is in suspension. In those embodiments were the cell is in suspension, it can be present at a density of at least 10⁴ cells/ml, at least 10⁵ cells/ml, at least 10⁶ cells/ml, or at least 10⁷ cells/ml. In one embodiment, clumping of the cells in suspension is minimized.

In some embodiments, the conditions used to assay a target protein may be modified to mimic the environment present in a pathological condition. For instance, the target protein may be expressed in a cancer cell, or a cell may be exposed to conditions that mimic a pathological condition. For instance, when using a cardiac cell the cell may be exposed to hypoxic conditions to mimic the conditions present during a myocardial infarction. In one embodiment, the cell may express one or more mutations that mimic a pathological condition. Other conditions vary depending on the type of cell and the type of pathologic condition to be mimicked, and such conditions are known in the art and routinely practiced by the skilled person.

The method further includes contacting a sample that includes the target protein with a test compound to form a mixture (e.g., block 320). The volume of the mixture can be at least 1 microliter (μL), at least 2 μL, at least 5 μL, at least 500 μL, or at least 1 milliliter (mL). The contacting can occur in the well of a multi-well plate that will be exposed to a light source, or the contacting can occur in a separate vessel and a portion of the mixture transferred to a well multi-well plate.

In certain embodiments, the methods provided herein are carried out in a well of a plate with a plurality of wells, such as a multi-well plate or a multi-domain multi-well plate. The use of multi-well assay plates allows for the parallel processing and analysis of multiple samples distributed in multiple wells of a plate. Multi-well assay plates (also known as microplates or microtiter plates) can take a variety of forms, sizes and shapes (for instance, round- or flat-bottom multi-well plates). Examples of multi-well plate formats that can be used in the methods provided herein include those found on 96-well plates (12×8 array of wells), 384-well plates (24×16 array of wells), 1536-well plate (48×32 array of well), 3456-well plates and 9600-well plates. Other formats that may be used in the methods provided herein include, but are not limited to, single or multi-well plates comprising a plurality of domains. In certain embodiments, the plates are opaque-wall, opaque-bottom plates. In certain embodiments, the plates are black-wall, black-bottom plates. In certain embodiments, the plates have black walls and clear bottoms in order to allow bottom excitation and reading of the fluorescence signals. In certain embodiments, the plates are chosen with minimal and uniform intrinsic fluorescence intensity within the range used in the method to avoid interference with the FRET signals.

A test compound useful in the method includes, but is not limited to, an organic compound, an inorganic compound, a metal, a polypeptide, a non-ribosomal polypeptide, a polyketide, or a peptidomimetic compound. The sources for test compounds that may alter activity of a protein described herein include, but are not limited to, chemical compound libraries, fermentation media of Streptomycetes, other bacteria and fungi, and cell extracts of plants and other vegetations. Small molecule libraries are available, and include AMRI library, AnalytiCon, BioFocus DPI Library, Chem-X-Infinity, ChemBridge Library, ChemDiv Library, Enamine Library, The Greenpharma Natural Compound Library, Life Chemicals Library, LOPAC1280™, MicroSource Spectrum Collection, Pharmakon, The Prestwick Chemical Library®, SPECS, NIH Clinical Collection, Chiral Centers Diversity Library. In some embodiments, the number of compounds evaluated in an assay includes between 1 and 200,000 compounds, between 1 and 100,000 compounds, between 1 and 1,000 compounds, or between 1 and 100 test compounds.

The mixture is exposed to a light source, and the resulting fluorescence emission spectrum is measured (e.g., block 320). The mixture can be incubated for any length of time before the exposure and measuring, such as at least 1 minute, at least 3 minutes, at least 10 minutes, at least 20 minutes, at least 60 minutes, at least 90 minutes, or at least 120 minutes. The measuring of the mixture can occur over a specific time period. In one embodiment, the time period of measuring the fluorescence emission spectrum of a mixture is no greater than 5 seconds, no greater than 1 second, no greater than 0.5 seconds, no greater than 0.1 seconds, no greater than 0.01 seconds, no greater than 0.001 seconds, no greater than 0.0001 seconds, no greater than 0.00001 seconds, or no greater than 0.000005 seconds.

The fluorescence emission spectrum is processed to decompose it into component spectra (e.g., block 410). In one embodiment, the number of component spectra is two; a donor chromophore emission and an acceptor chromophore emission. In another embodiment, the number of component spectra is four; a donor chromophore emission and an acceptor chromophore emission, a water Raman emission, and a cell autofluorescence emission. In one embodiment, the fluorescence emission spectrum is decomposed by fitting the component spectra to a linear model, determining the contribution of each signal, and using the shape of each component spectra to decompose the fluorescence spectrum. In one embodiment, the fluorescence emission spectrum is decomposed according to

F_{Fit}(λ)=aF_{D}(λ)+bF_{A}(λ)+cF_{C}(λ)+dF_{W}(λ)

wherein where D is donor, A is acceptor, C is cell autofluorescence, and W is water Raman, and a, b, c, d are the weighting (scalar) coefficients determined from the fit. Those embodiments where two-component spectra are used typically to determine the contributions of the donor and acceptor are when the fluorescence signal of the acceptor and donor components are very intense in comparison to the background signal contribution from cellular autofluorescence and water Raman. Additional component spectra that can be decomposed include, but are not limited to, sample contamination, sample degradation, or fluorescence of the plate.

The ratio (referred to herein interchangeably as FR or R) for the decomposed fluorescence emission spectrum is calculated (e.g., block 420). The R is calculated by determining the total fluorescence from the acceptor chromophore and the total fluorescence from the donor chromophore. In one embodiment, R is determined according to

$$R = \frac{Acceptor fluorescence}{Donor fluorescence} = \frac{{bF}_{A}}{{aF}_{D}} .$$

A full derivation of this equation is described in Examples 1 and 2.

In one embodiment, the coefficient of variation (CV) of R obtained from a mixture (e.g., a mixture present in a well) is no greater than 3%, no greater than 1%, no greater than 0.5%, or no greater than 0.3%.

R is used to determine whether the test compound present in one of the mixtures alters the FRET of the target protein (e.g., block 510). The R of the mixture with the test compound is compared to the R of a mixture that does not include the text compound (e.g., a baseline). A difference between the R in the presence of the test compound and the R in the absence of the test compound indicates that the test compound alters the FRET of the target protein. The difference can be at least 0.5%, at least 1%, or at least 5%. An altered FRET suggests that the test compound alters the environment around the chromophore, which in turn suggests the structure (conformation) of the protein has changed in response to the test compound. The altered structure may be a change in the secondary structure, the tertiary structure, or a combination thereof. An altered FRET is the result, for instance, of a change in the structure of the target protein, a change in ligand-binding by the target protein, or a combination thereof. An alteration in FRET may alter an activity of the target protein, such as an enzymatic activity, or the ability to bind to a ligand, such as an accessory protein. An alteration in FRET may be due to the test compound acting as an activator or an inhibitor of a target protein.

In some embodiments, the value for FR can be used to calculate the FRET efficiency for the mixture (e.g., block 430). In one embodiment, calculating the FRET efficiency includes using the extinction coefficient, quantum yield of the donor chromophore and acceptor chromophore, correcting for spectrometer sensitivity, or a combination thereof. In one embodiment, the FRET efficiency is determined according to

$$FRET = \frac{R \times QR - AR}{1 + R \times QR}$$

where QR is the ratio of quantum yields (Q_{D}/Q_{A}) in the absence of FRET, AR is the ratio of molar absorptivity (ε_{A}/ε_{D}), both obtained from reported values (Lambert and Thorn, 2016, available on the World Wide Web at nic.ucsf.edu/FPvisualization). QR can be, and preferably is, corrected for spectrometer sensitivity at the appropriate wavelengths. In one embodiment, calculation of FRET efficiency values can be useful when intermolecular FRET is being evaluated.

Optionally, fluorescent compounds can be identified and flagged as potential false-positives by evaluating the similarity index (SI). Interference from fluorescent compounds may alter the shape of the fluorescence emission spectrum in an unpredictable manner. For instance, when GFP and RFP are used, fluorescent compounds (such as test compounds) that emit at wavelengths similar to GFP and RFP can change the FRET efficiency, fluorescence ratio, or lifetime in a manner that appears to be a potential hit. These fluorescent compounds may artificially alter the donor lifetime or acceptor/donor ratio (fluorescence ratio R) from the FRET biosensor, giving the appearance of a FRET hit, but this alteration is not due to FRET. The similarity index is used to eliminate these potential fluorescent compounds from the screen, and focus the attention on the test compound wells, which have emission spectrum and lifetime values, that are due to changes in the FRET of a particular biosensor, and not interference from fluorescence compounds.

In general, the similarity index of each test compound well of a particular screen in comparison to a control spectrum (averaged over the DMSO control wells) is determined from the fluorescence emission spectrum. Test compounds wells that significantly alter FRET of a particular biosensor are identified by either lifetime or spectral fitting. If these test compound wells (FRET hits) are determined to have a similarity index (shape of emission spectrum from GFP, RFP, etc.) that is consistent with that expected from a FRET biosensor, the compound is considered to alter FRET. The similarity index is incredibly sensitive to small changes in the fluorescence emission spectrum that may not be detected by spectral and lifetime fitting. For example, a false-positive hit (test compound well), may be due to a slightly fluorescence compound that has a lifetime slightly above or below the donor chromophore, or a compound with an emission spectrum slightly different in shape from that of the donor or acceptor. As described in Example 2, the similarity index was used to detect fluorescent compounds, but it could also be used to detect fluorescence interference from sample contamination, preparation error, dispensing error, or other unknown sources of fluorescence interference.

In one embodiment, SI is evaluated between an observed compound spectrum I^{(a)} and control or baseline spectra I^{(b)} that includes the same carrier used to dilute the test compound, e.g., DMSO. The SI can be determined according to

$$SI = 1 - \text{?}$$

$$\text{?} \text{indicates text missing or illegible when filed}$$

The spectra of a plurality of DMSO control wells (% v/v DMSO) are averaged to generate a single control spectrum (I^{(b)}). The fluorescence spectra of the target protein (if the target protein includes both donor and acceptor chromophores) or the target protein and second protein, screened against a set of N separate test compounds (I^{(a} [symbol] ⁾), is compared with the single DMSO control spectrum. The similarity index between the spectra can be computed over the entire emission spectrum or for specific regions where interference from fluorescent compounds is more likely (dependent on the signal of donor and acceptor, or amount of FRET efficiency of a particular biosensor) such as those of the donor chromophore. For instance, if the donor is GFP, the emission spectrum is from 500-540 nm (i=500-540 nm). The resulting SI value is a unitless quantity, typically between 0 and 1, where numbers closer to 0 are considered more similar.

A threshold SI value for can be determined, where test samples having an SI value above that threshold are predicted to be false positives. A threshold value can vary depending upon the target protein used, the assay conditions, and other variables. In one embodiment, the results from an assay can be graphed with the SI of each well as a function of the ranked order of the wells, from lowest SI to highest. This provides a visual representation allowing the skilled person to easily identify those wells having a substantially higher SI than most other wells, and the threshold value can be established. In another embodiment, the threshold value is at least 0.000001, at least 0.00001, at least 0.0001, at least 0.001, at least 0.01, at least 0.1, at least 0.2, at least 0.3, at least 0.4, or at least 0.5. In another embodiment, the threshold is a value greater than 1 standard deviation, greater than 2 standard deviations, or greater than 3 standard deviations of the normal distribution of all test compounds. For instance, in Examples 2 The SI threshold was determined by evaluating the distribution of SI values of all wells containing test compounds. It was found that approximately 5-10% of all the test compound wells significantly deviated from the normal distribution at 2×10⁻⁴. The shape of the spectrum from the compound test wells, from the majority of the test compound wells were similar to one another, and only the wells that had a value above 0.0002 were found to be significantly different in comparison to the DMSO control wells. The test compound wells which exhibited a SI value above 0.0002 were excluded from further analysis of spectral and lifetime fitting, as the shape of their emission spectra was dissimilar to the control.

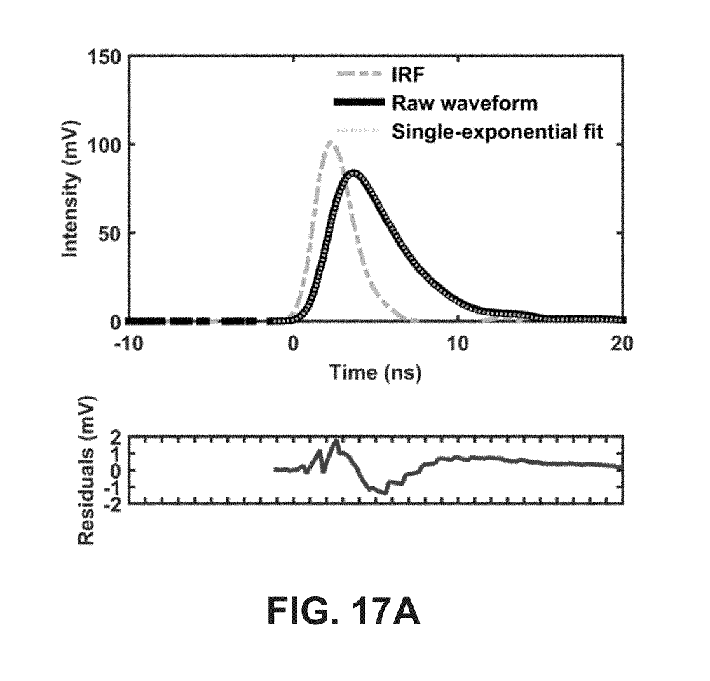

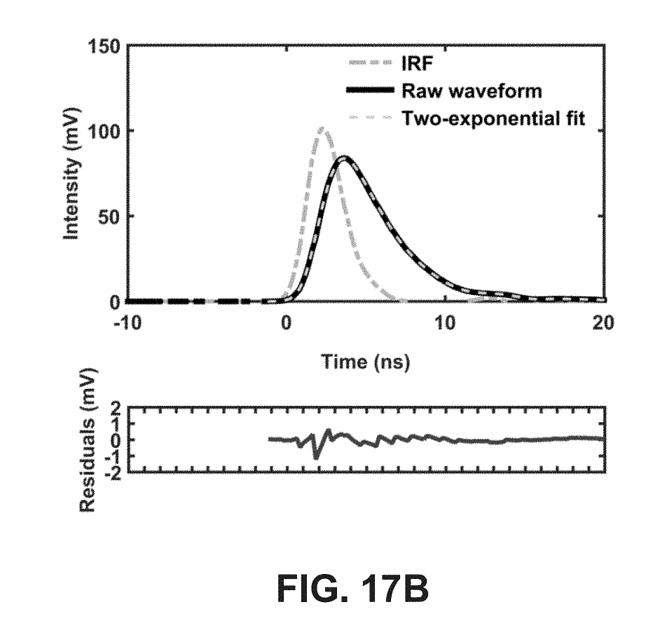

In one embodiment, global analysis of the data resulting from scanning in lifetime mode can be used to resolve distance distributions and mole fractions of structural states of a target protein that includes a donor chromophore and an acceptor chromophore. Using the data from the fluorescence lifetime scan and the Forster distance for the FRET pair, distinct structural states can be deduced [defined by j in Eq 2-6]. This provides the number of structural states, their interprobe distances and the widths of the distributions, and the fraction of molecules in each state. No other knowledge about the protein's structure is required. The distance distributions and mole fractions of structural states of a target protein can be determined according to

$$\begin{matrix}\text{?} & ( Eq . 1 ) \\ \text{?} & ( Eq . 2 ) \\ F ( t ) = \text{?} & ( Eq . 3 ) \\ \text{?} ( t ) = \text{?} ( R ) \text{?} & ( Eq . 4 ) \\ \text{?} & ( Eq . 5 ) \\ \text{?} \\ \text{?} \text{indicates text missing or illegible when filed} & ( Eq . 6 )\end{matrix}$$

where F_{D} is the time-resolved fluorescence decay function of the donor (Eq. 1), best-fit by a two exponential decay (see Examples 1 and 2, FIGS. 17A-B). F_{DA} (Eq. 2) is the time-resolved fluorescence decay function of the donor-acceptor FRET pair. F_{D} and F_{DA} are fit to a linear combination of mole fractions of x_{D} and x_{DA} and x_{D} equals zero for the intramolecular FRET sensor (Eq. 3). F_{DA} is a linear combination with molar fraction Xⱼ of two FRET-affected fluorescence decays Tⱼ(t) (Eq. 4). ρⱼ is the probability of each distance distribution, determined by least-squares minimization of the distance (nm) R associated with each donor-acceptor lifetime species τᵢ (Eq. 5). The center Rⱼ of a Gaussian interprobe distance distribution ρⱼ(R) can be identified with distribution widths defined by the standard deviation σⱼ and full-width half-maximum FWHMⱼ (Eq. 6). For instance, when the FRET pair was GFP and RFP, Gaussian interprobe distance distributions (ρⱼ) centered at Rⱼ=5.5 nm and 10.2 nm (See Example 2). The Förster distance (R₀) for the eGFP and tagRFP FRET pair is 5.8 nm. The parameters in this system of equations can be optimized utilizing simultaneous least-squares minimization to waveforms from donor-only and donor-acceptor cell lines. The best-fit model can be indicated by minimized χ² and by evaluation of the parameter error surface as described (Muretta et al., Proc Natl Acad Sci USA 2015, 112, E6606-6613; Muretta et al., Proc Natl Acad Sci USA 2015, 112, 14272-142771; Li et al., J Mol Biol 2012, 418, 379-389).

### Definitions

As used herein, the term “protein” refers broadly to a polymer of two or more amino acids joined together by peptide bonds. The term “protein” also includes molecules that contain more than one protein joined by disulfide bonds, ionic bonds, or hydrophobic interactions, or complexes of polypeptides that are joined together, covalently or noncovalently, as multimers (e.g., dimers, tetramers). Thus, the terms peptide, oligopeptide, and polypeptide are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, the terms “FRET,” “fluorescence resonance energy transfer,” “Førster resonance energy transfer,” “fluorescence energy transfer,” and “resonance energy transfer” are used interchangeably, and refer to a nonradiative energy transfer process that occurs between two chromophores.

As used herein, a “chromophore” is a molecule that includes a region that adsorbs certain wavelengths of light and interacts with such a region of another chromophore so as to be useful for FRET. Chromophores suitable for use in a FRET assay are known to the skilled person and are readily available. In one embodiment, a chromophore may be a donor (also referred to as a donor probe and donor chromophore). A donor probe refers to a molecule that will absorb energy and then re-emit at least a portion of the energy over time. In one embodiment, a chromophore may be an acceptor (also referred to as an acceptor probe and acceptor chromophore). An acceptor probe refers to a molecule that will accept energy nonradiatively from a donor, thus decreasing the donor's emission intensity and excited-state lifetime. A donor probe and acceptor probe that interact in this way are referred to herein interchangeably as a donor-acceptor pair or a FRET pair. Thus, provided that a donor probe and an acceptor probe are physically located sufficiently close (most often within 2.5 to 12 nm), the two probes function together and, upon excitation with an appropriate wavelength, the donor probe transfers a precise amount of energy (proportional to the negative sixth power of the donor-acceptor distance) to the acceptor probe. This process can be specifically and quantitatively detected by observing the decrease in donor fluorescence intensity or lifetime or, in some cases, also the energy re-emitted by the acceptor probe as fluorescence. Thus, FRET assays are typically used to measure (1) the mole fraction of donors coupled with acceptor (e.g., to determine the binding affinity between the donor-labeled and acceptor-labeled molecules) and (2) the distance and/or distance changes between donor and acceptor. When donor and acceptor are both attached to the same molecule, FRET can be used to detect a change in the molecule's structure. When donor and acceptor are attached to different molecules, FRET can be used to detect a change in the relative positions (e.g., binding, orientation) and structures of the two molecules.

As used herein, the term “high-throughput screening” or “HTS” refers to a method drawing on different technologies and disciplines, for example, optics, chemistry, biology or image analysis, to permit rapid analysis of multiple samples at rates that permit highly parallel biological research and drug discovery. In one embodiment, an HTS method includes the use of samples having a volume of at least 2 microliters (μl), or at least 4 μl.

As used herein, “activity” or “biological activity” of a protein refers to a function of protein in a cell. An activity of a protein in a cell may include binding to another molecule (e.g., an interaction between two subunits of a multimeric protein, or an interaction between a target protein and a non-protein ligand), an enzymatic activity, or the combination thereof.

As used herein, the term “wild-type” refers to the most typical form of an organism, protein, or characteristic as it occurs in nature.

As used herein, “genetically engineered cell” and “genetically modified cell” are used interchangeably and refer to cell into which has been introduced an exogenous polynucleotide and has been altered by human intervention. A cell is a genetically engineered cell by virtue of introduction of an exogenous polynucleotide that encodes a target protein described herein. In one embodiment, the genetically engineered cell includes more than one exogenous polynucleotide. In one embodiment, the genetically engineered cell stably expresses a target protein, for instance, the exogenous polynucleotide is not diluted through mitosis and/or degraded (expression of the target protein is not transient).

As used herein, “coefficient of variation” (CV) refers to a normalized measure of dispersion of a probability distribution or frequency distribution, and is defined as the ratio of the standard deviation to the mean.

The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The present disclosure is illustrated by the following examples. It is to be understood that the particular examples, materials, amounts, and procedures are to be interpreted broadly in accordance with the scope and spirit of the disclosure as set forth herein.

### Example 1

### Spectral Unmixing Plate Reader: High-Throughput, High-Precision FRET Assays in Living Cells

Described here is a microplate reader that records a complete high-quality fluorescence emission spectrum on a well-by-well basis under true high-throughput screening (HTS) conditions. The read time for an entire 384-well plate is less than 3 minutes. This instrument is particularly well suited for assays based on fluorescence resonance energy transfer (FRET). Intramolecular protein biosensors with genetically encoded GFP donor and RFP acceptor tags at positions sensitive to structural changes were stably expressed and studied in living HEK cells. Accurate quantitation of FRET was achieved by decomposing each observed spectrum into a linear combination of four component (basis) spectra (GFP emission, RFP emission, water Raman, and cell autofluorescence). Excitation and detection are both conducted from the top, allowing for thermoelectric control of the sample temperature from below. This spectral unmixing plate-reader (SUPR) delivers an unprecedented combination of speed, precision, and accuracy for studying ensemble-averaged FRET in living cells. It complements our previously reported fluorescence lifetime plate reader, which offers the feature of resolving multiple FRET populations within the ensemble. The combination of these two direct waveform-recording technologies greatly enhances the precision and information content for HTS in drug discovery. This Example is also available as Schaaf et al., SLAS Discov. 2016 Mar. 22(3): 250-261. [Note: The journal was published from 1996 through 2016 with the title Journal of Biomolecular Screening. Its name changed in 2017 to SLAS Discov. The older citation prior to this change includes: Schaaf et al., J Biomol Screen. 2016 Nov. 22. pii: 1087057116679637.]

### Introduction

Numerous live-cell FRET biosensors based on genetically encoded fluorescent fusion proteins have been developed, but their application in high-throughput screening (HTS) assays is uncommon. An intramolecular FRET sensor for sarco/endoplasmic reticulum calcium ATPase (SERCA) [1-3] is a rare exception [3]. This two-color SERCA (2CS) biosensor, expressed in HEK293 cells, employs eGFP and tagRFP (further referred to as GFP and RFP) fluorescent proteins [4]. Fluorescent proteins were engineered at carefully selected locations on SERCA's cytoplasmic headpiece domains. The headpiece domains are known to undergo large-scale structural changes as ATP hydrolysis fuels the active pumping of calcium from the cytosol into the endoplasmic reticulum. The interactions of small molecules with SERCA produce measurable changes in intramolecular FRET that correlate with function, making this structure-based biosensor a powerful tool for the discovery of novel drugs related to calcium homeostasis.

The previous development of a novel fluorescence lifetime readout in the nanosecond time domain was shown to enable rapid and reliable identification of small-molecules that affected FRET in the two-color SERCA (2CS) biosensor [3, 5-7]. Conventional microplate readers, which measure fluorescence intensity, lack the precision required for reliable observation of the typically small FRET changes associated with allosteric effectors in live-cell assays. Similarly, the throughput of related fluorescence technologies such as microscopy is too low, and the precision of flow cytometry is currently too low for large-scale library screening [8, 9]. The technological advances of direct waveform recording (DWR) led to the development of a novel fluorescence lifetime plate reader in the time domain [5], and now have been applied to the wavelength-domain. This new spectral unmixing microplate reader rapidly and accurately records the entire fluorescence emission spectrum.

The quality of the acquired spectral data enables a simple and direct decomposition of the observed spectra into linear combinations of component spectra (spectral unmixing), yielding accurate and precise FRET efficiency values. The benefits are critically evaluated by coupling fluorescence lifetime detection with the complementary spectral recording to further optimize the precision of FRET measurements for high-throughput screening. The combination of the spectral and fluorescence lifetime readouts represents a promising screening platform for detecting small changes in FRET.

This study emphasizes live-cell biosensors, but the same approach is equally applicable to purified proteins labeled with dyes [10]. In addition to drug discovery activities, the spectral recording technology has high potential for a wide range of biological applications, such as binding studies, analytical biochemistry, and molecular diagnostics.

### Materials and Methods

### Cell Culture

HEK293 cells were maintained in phenol-red-free DMEM from Gibco (Waltham, Mass.) supplemented with 2 mM GlutaMAX (Gibco), 10% fetal bovine serum (FBS) from Atlanta Biologicals (Laweranceville, Ga.), and 1 IU/mL penicillin/streptomycin (Gibco) and grown at 37° C. with 5% CO₂. Stable clones expressing the FRET biosensors and corresponding donor- and acceptor-labeled control cell lines were established as described previously [3]. Briefly, cells were transfected with the recombinant DNA following established protocols lipofectaimine 3000 from Invitrogen (Carlsbad, Calif.). 48 hours post transfection the cells were placed under G418 (500 μg/mL) from Sigma (St. Louis, Mo.), antibiotic selection, and plated to allow for the growth of single colonies. Clones were isolated 2-3 weeks after transfection and antibiotic selection. The stability of each clone was assessed by flow cytometry and confocal microscopy (data not shown). The cell lines were expanded in T225 flasks from Corning (Corning, N.Y.), harvested by treatment with Tryple (Invitrogen), washed three times in phosphate buffer solution (PBS) with no magnesium or calcium Thermo Fischer (Waltham, Mass.)) and centrifuged at 300 g, filtered using 70 μm cell strainers (Corning), and diluted to 10⁶ cells/mL using an automated countess cell counter from Invitrogen. In studies with cyan and yellow fluorescent protein biosensors, transient transfections were performed. In these studies, cells were harvested and prepared as described above, 48 hours after transfection. Cell viability was assessed using trypan blue.

### Cell and Drug Liquid Dispensing

Cells were dispensed using a Multidrop Combi liquid dispenser from Thermo Fischer (Pittsburgh, Pa.) at a density of 10⁶/mL. Compounds were diluted in DMSO and dispensed either using a Mosquito liquid handler from TTP Labtech (Melbourn, UK) or a Mantis liquid dispenser from Formulatrix (Bedford, Mass.). The known SERCA inhibitors thapsigargin (TG, Sigma), 1,4-dihydroxy-2,5-di-tert-butylbenzene (BHQ) from Tocris (Minneapolis, Minn.), and cyclopiazonic acid (CPA, Tocris) were diluted at 50× concentrations and subsequently serially diluted in 96-well mother plates prior to liquid dispensing. Cells and drug mixtures were dispensed into 384-well flat, black-bottom polypropylene plates from Greiner (Kremsmünste, Austria) and incubated for 20 min at room temperature (20-23° C.), unless otherwise noted.

### Instrumentation Overview

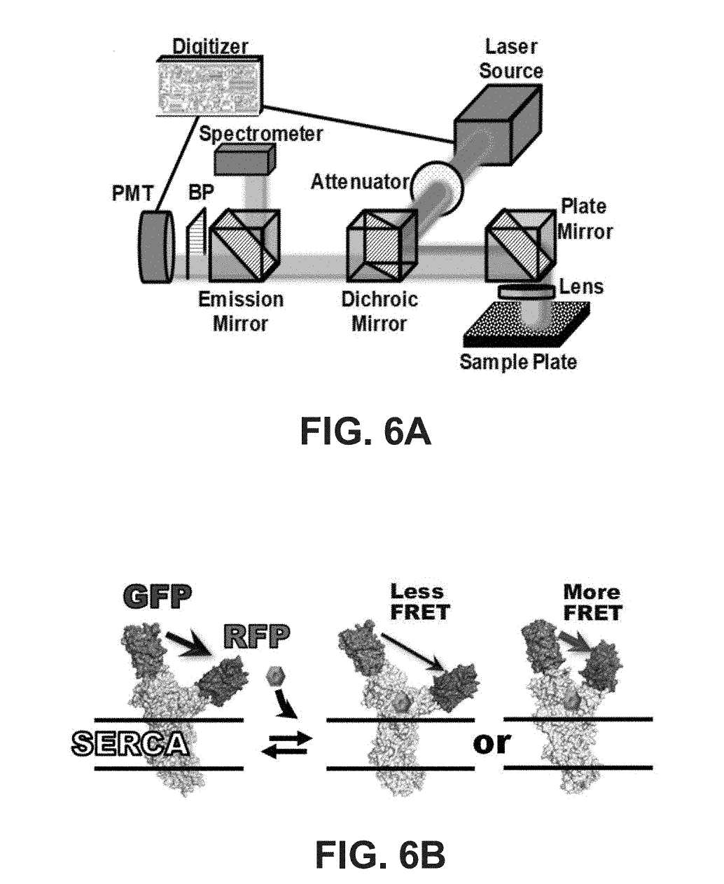

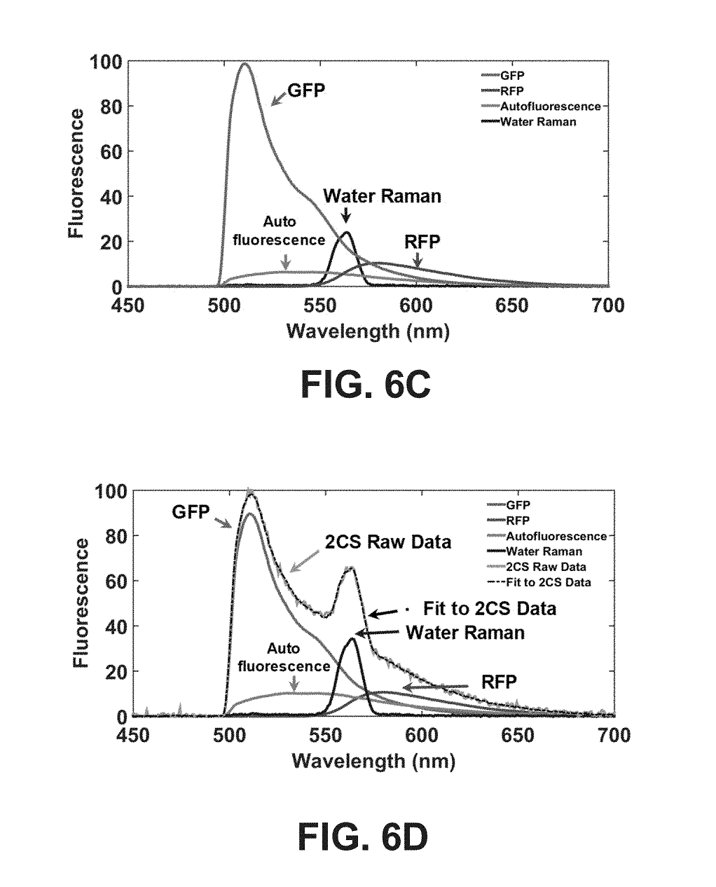

FIG. 6A is a schematic drawing depicting key features of the fluorescence plate-reader platform. The lifetime and spectral readout experiments presented here were conducted with separate instruments at 20° C., but both modes can be incorporated into a single instrument, and work along these lines is in progress. Simultaneous acquisition of the lifetime and spectral data is also feasible. The photomultiplier tube (PMT) and digitizer are as described previously [5], but this new instrument employs epi-illumination excitation and detection from above, which facilitates implementation of temperature control.

In its spectral unmixing plate reader (SUPR) mode, the instrument provides direct high-throughput detection of the complete fluorescence emission spectrum, (emission vs wavelength), with excitation provided by a 473 nm continuous wave laser. Spectra are recorded using a grating-based fiber optic input spectrograph equipped with linear-array CCD detector (Sony ILX511B). The recorded wavelength range in these experiments spanned the entire visible spectrum, but only the 400-700 nm range was used in the data analysis. In lifetime mode, the full nanosecond-resolved fluorescence emission waveform is acquired following excitation with a 473 nm pulsed microchip laser. The acquisition time per well is typically 200 ms in either spectral or lifetime mode (FIG. 6A).

### Time-Resolved FRET Acquisition and Analysis

Fluorescence decay waveforms for lifetime determination, were detected directly as previously described [3, 5]. The 473 nm passively Q-switched microchip laser (Concepts Research Corporation, Belgium, Wis.) delivers highly reproducible, and high-energy pulses (˜1 μJ) at 5 kHz repetition rate. A full fluorescence decay waveform was detected in response to each laser pulse over a 128 ns time window, using a photomultiplier module from Hamamatsu (Cat# H10720-210), and a proprietary transient digitizer from Fluorescence Innovations (Minneapolis, Minn.). A 488 nm long-pass filter from Semrock (Rochester, N.Y.) and 517/20 bandpass emission filter (lifetime mode), were used, ensuring that only emission from the GFP donor was detected. A 488 nm dichroic mirror directed fluorescence signal toward the PMT (lifetime mode) or spectrograph (spectral mode) using a fiber optic cable (FIG. 6A).

The observed donor fluorescence waveform F_{D}(t) was analyzed using least-squares minimization global analysis software [5] and fitted (Eq. 7) by a simulation S_{D}(t), consisting of a n-exponential decay model M_{D}(t) (characterized by pre-exponential factors Aᵢ and lifetimes τ_{Di}), convolved with the instrument response function IRF(t), acquired by recording scatter from 0.31 μm latex microsphere suspensions (Thermo Fischer).

$${M}_{D} ( t ) = {\sum }_{i = 1}^{n} {A}_{i} exp ( - t / {τ}_{Di} ) , \\ {S}_{D} ( t ) = {\int }_{- ∞}^{+ ∞} IRF ( t - {t}^{′} ) {M}_{D} ( {t}^{′} ) {dt}^{′} \qquad ( Eq . 7 )$$

For initial analysis, a single-exponential model (n=1) was assumed, and FRET (efficiency) was calculated from (Eq. 8).

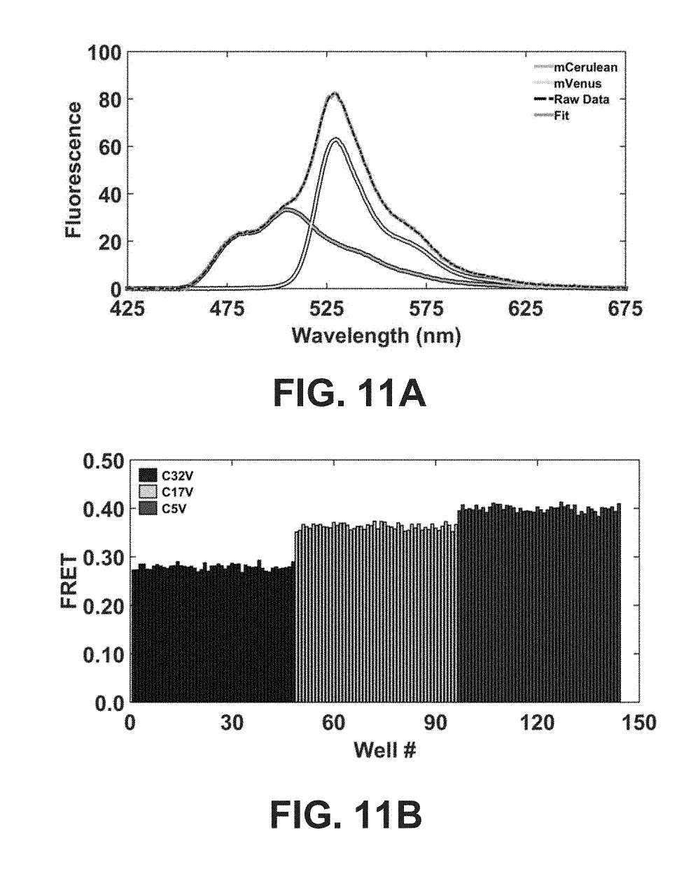

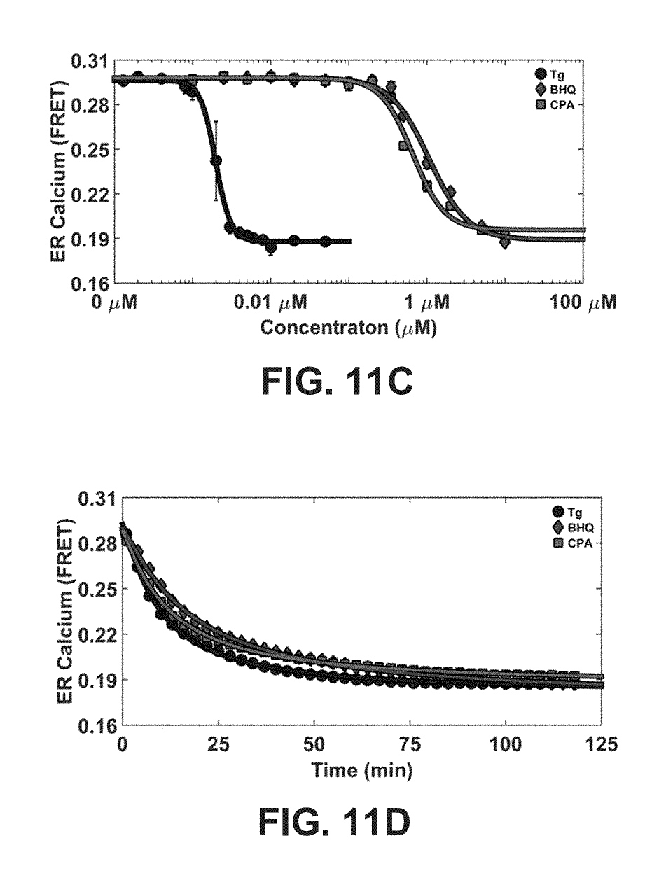

FRET=1−(τ_{DA}/τ_{D}) (Eq. 8)

Where τ_{DA} is the lifetime of the intramolecular FRET biosensor, and τ_{D} is the lifetime of the corresponding donor-only cell line. The fluorescence lifetime of eGFP from a single-exponential fit of the donor-only control was 2.58±0.02 ns (FIGS. 11A-D), in agreement with reported values [11].

### Spectral FRET Acquisition and Analysis

The fluorescence spectra were recorded with a fiber-optic spectrometer. Commercially-available spectrometers that may be used are: Ocean Optics Fiber Optic Fluorescence Spectrometer 360-1000 nm, Ibsen Photonics (ROCK VIS) 380 to 770 nm, Avantes AvaSpec, ULS2048L StarLine Versatile Fiber-optic Spectrometer 200-1100 nm range, Stella Net Inc. Black Comet High Resolution Concave Grating Spectrometer 380-750 nm, PemBroke Instruments Qmini VIS 370-750 nm. These uncooled linear array detector offers higher data density (pitch) compared to the multimode PMTs, that have more commonly been used for spectral unmixing in fluorescence microscopy [12]. The linear array detector is also substantially smaller, and less costly compared to a TE-cooled 2-dimensional scientific grade CCD camera.

The observed fluorescence emission spectrum F(λ) was fitted by least-squares minimization to a linear combination of component spectra

F_{Fit}(λ)=aF_{D}(λ)+bF_{A}(λ)+cF_{C}(λ)+dF_{W}(λ) (Eq. 9)

where D is donor, A is acceptor, C is cell autofluorescence, and W is water Raman, and a, b, c, d are the weighting (scalar) coefficients determined from the fit. The fitted spectrum for each well was determined using least squares minimization with Matlab (Mathworks) to solve for the scalar coefficients.

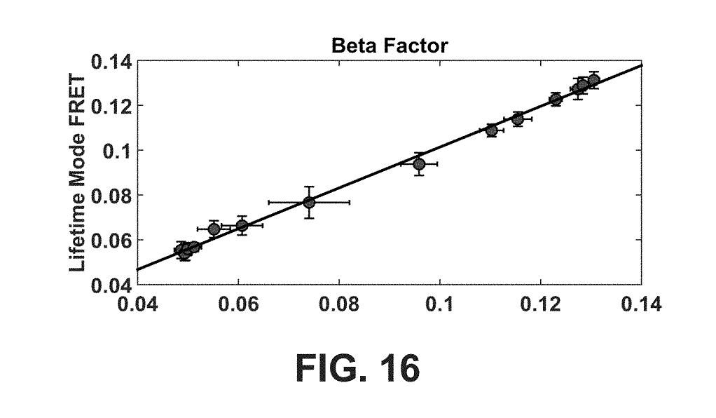

For an intramolecular FRET sensor with a 1:1 ratio of donor D and acceptor A molecules, FRET efficiency (FRET) was determined from (Eq. 10).

$$FRET = \frac{FR \times QR - AR}{1 + FR \times QR} , \qquad ( Eq . 10 )$$

Where QR is the ratio of quantum yields (Q_{D}/Q_{A}) in the absence of FRET, AR is the ratio of molar absorptivity (ε_{A}/ε_{D}), both obtained from reported values [13]. QR was corrected for spectrometer sensitivity at the appropriate wavelengths (FIG. 16). The only experimentally observed variable in (Eq. 10) becomes the fluorescence ratio (FR).

$$FR = \frac{Acceptor fluorescence}{Donor fluorescence} = \frac{{bF}_{A}}{{aF}_{D}} , \qquad ( Eq . 11 )$$

A full derivation of (Eq. 10) is in Supplementary Data.

### Results

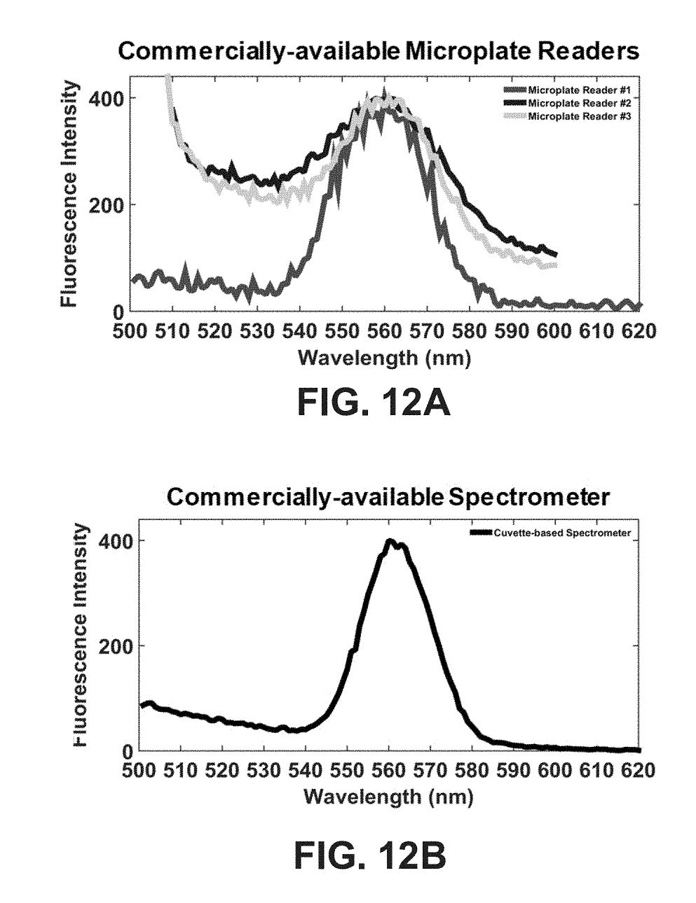

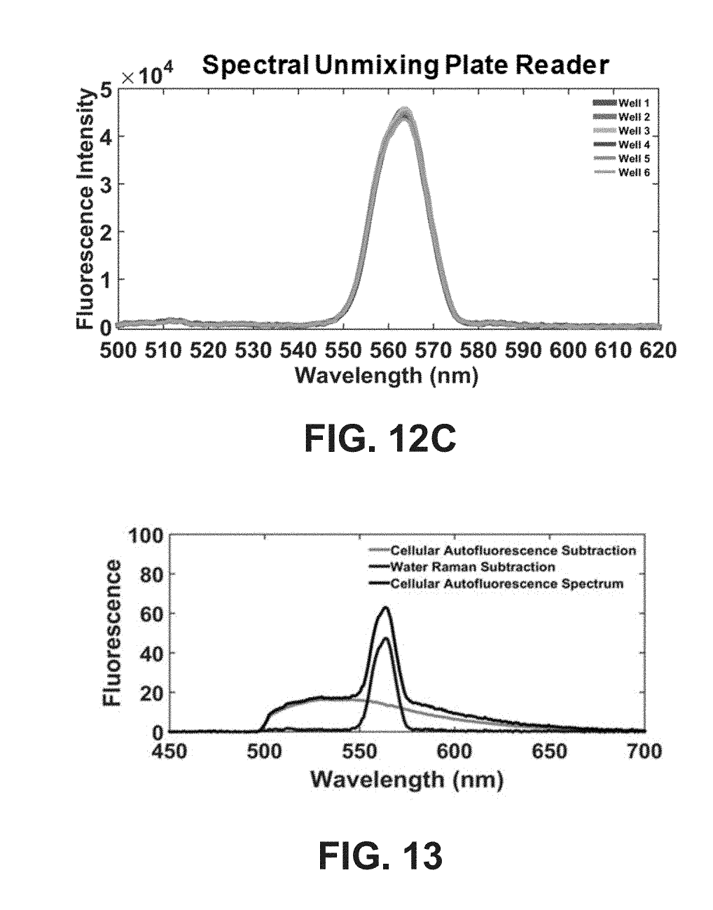

Two one-color stable cell lines expressing GFP-SERCA or RFP-SERCA were developed as controls for analysis of 2-color FRET spectra. Reference (basis) spectra from these cell lines, untransfected cells (water Raman plus cell autofluorescence), and buffer solution (water Raman) were acquired using a detector integration time of 100 ms, for each well of a 384-well plate. The GFP-only reference spectrum was corrected for autofluorescence and water Raman contributions (FIGS. 12A-C, FIG. 13). The RFP-only spectrum was acquired by excitation at 532 nm (FIG. 15), under which conditions the Raman and autofluorescence signals were negligible. Each of the four reference spectra was quite reproducible, so that a single set of reference spectra, acquired only one time, was used to decompose or unmix the component spectra from the observed fluorescence emission spectrum in 2CS samples for over six months. Reference spectra are superimposed in FIG. 6C, normalized to the peak intensity of the GFP sample. The RFP spectrum shown corresponds to the intensity observed with excitation at 473 nm. Spectral unmixing (Eq. 9) resolved the distinct components of GFP and RFP for quantitative determination of the apparent FRET (Eq. 10), (Eq. 11). FIG. 6D illustrates the analysis of a sample of cells expressing the 2CS biosensor, showing the observed spectrum (orange) and the best fit (dashed black) to the four components, yielding a FRET value of 13.7% (SD=0.11%).

### Spectral Unmixing of GFP and RFP Live-Cell Mixtures

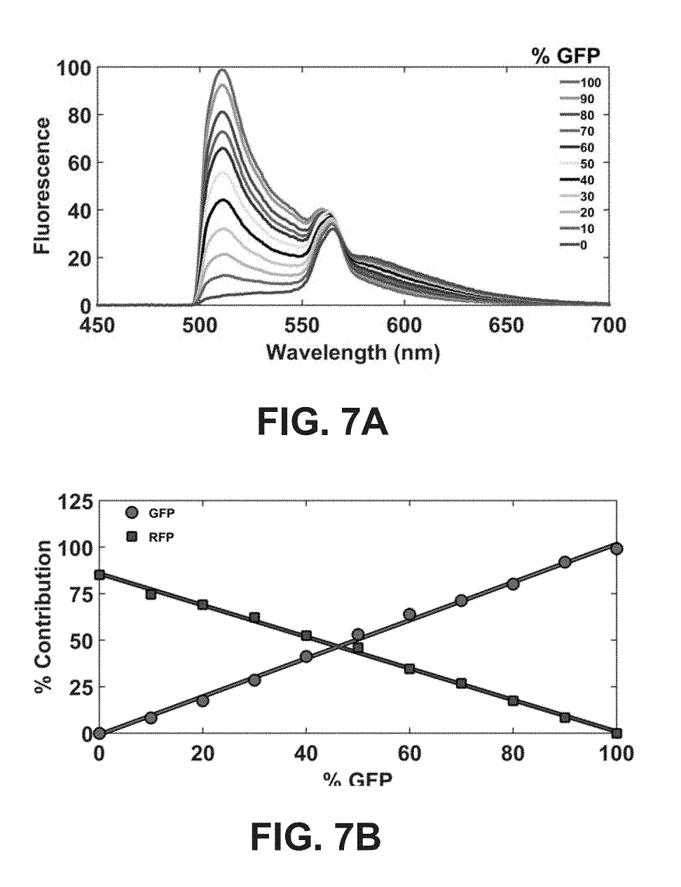

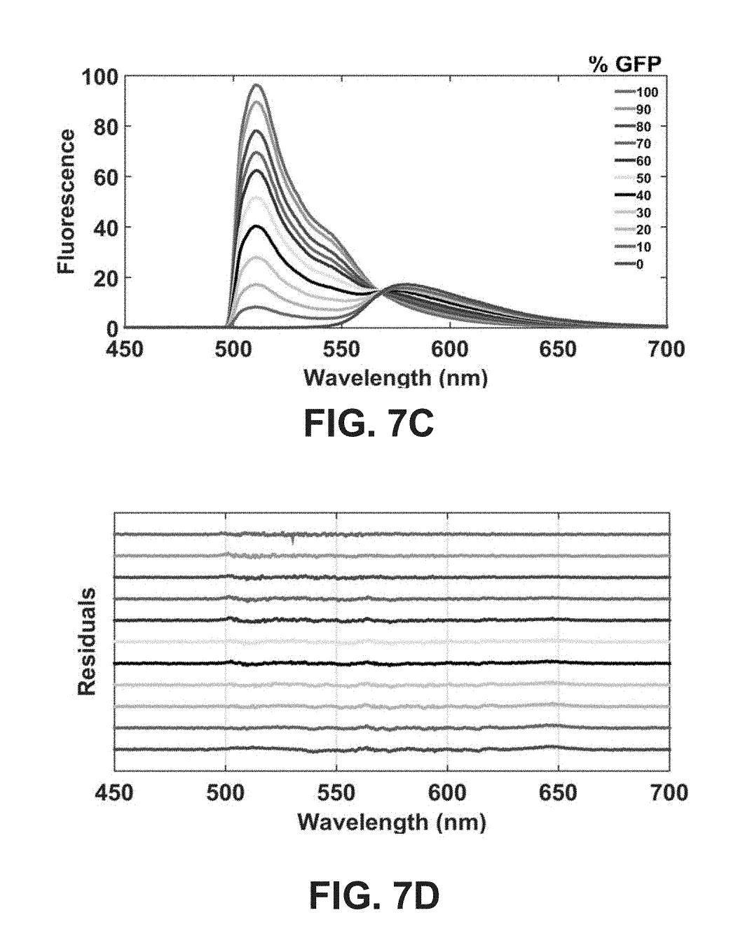

Accuracy and precision of the spectral unmixing method was verified by analyzing known mixtures of GFP- and RFP-expressing HEK293 cell lines, dispensed at 10⁶ cells/mL into a 384-well plate. The GFP-only control cell line was mixed with cells expressing the RFP-only control construct to produce wells with the percentage of GFP increasing in 10% increments. The total volume per well was 50 μL. The expression of the RFP-only cell line was five times that of the GFP-only cell line, as determined by quantitative western blotting. The observed spectra (raw data) show a uniform decrease in the GFP contribution (500-550 nm region) (FIG. 7A).

The individual scalar coefficients from the fluorescence emission spectrum of each mixture were assessed as described in (Eq. 9). The scalar coefficients of GFP (D) and RFP (A) were converted to fluorescence signal by multiplying by the total emission from the corresponding reference spectra (F_{RFP} and F_{GFP}) using (Eq. 10), (Eq. 11). The contributions of GFP (D) and RFP (A) increased inversely and linearly as expected. The contribution from cellular autofluorescence and water Raman remained constant across the GFP/RFP live-cell gradient (FIG. 7B) and were subtracted from the spectrum of each mixture (FIG. 7C). An isoemissive point at 567 nm, clearly distinguishes the change in GFP and RFP emission across the gradient of cellular mixtures. The residuals obtained using the four-component model (Eq. 9) are plotted for each % GFP mixture in FIG. 7D. The residuals remain flat, demonstrating excellent agreement between the fit and observed spectrum (Eq. 9).

The scalar coefficients were used to determine the contribution of each component to the total fluorescence signal. At the lowest mixture of cells expressing GFP (10% GFP), there are approximately 5,000 GFP-expressing cells and 45,000 RFP-expressing cells per well (50 μL total volume per well). Based on the diameter of the laser beam (500 microns), well dimensions, and diameter of one cell, we estimate approximately 500 cells expressing GFP contribute to the observed spectrum. As such, we have performed preliminary studies with low-volume 1536-well PCR plates, which indicate that decreasing the total volume and number of cells by a factor of ten, does not significantly degrade the data quality (results not shown).

Concentration-Response Curve Comparison of 2CS FRET Change with Known SERCA Inhibitors

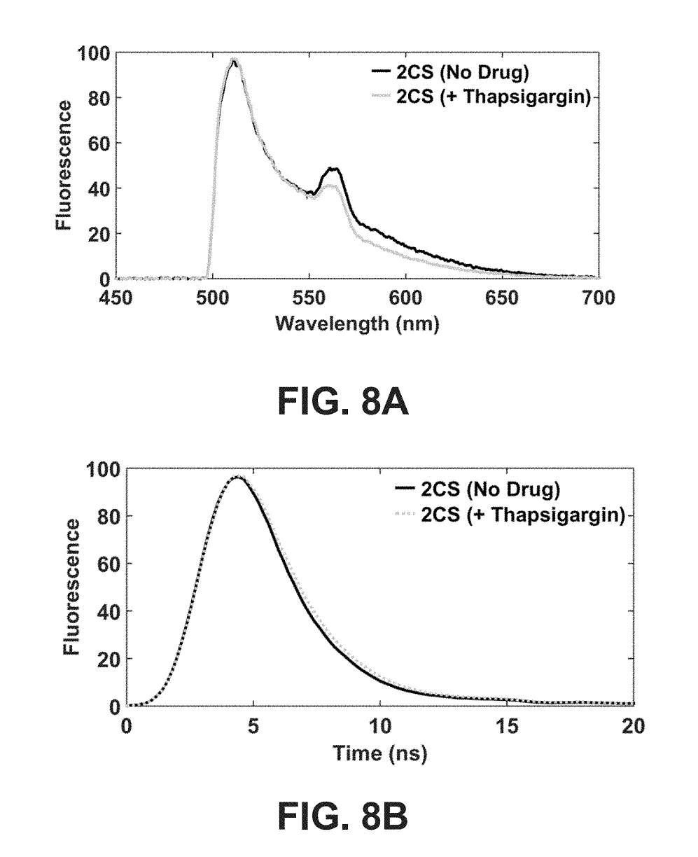

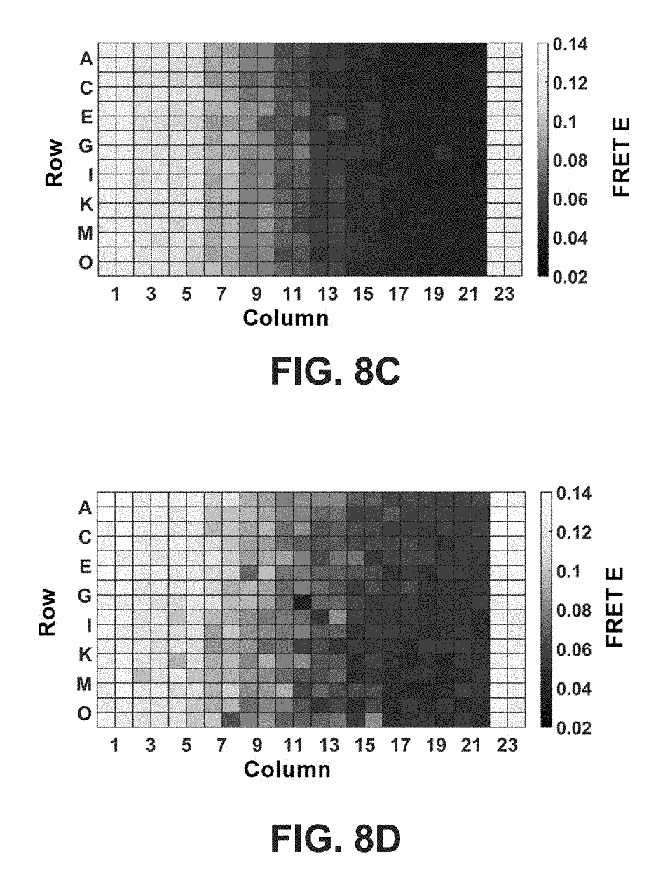

The spectral unmixing method's capability to resolve 2CS FRET changes via controlled addition of the known SERCA inhibitor thapsigargin, which binds to SERCA2a with subnanomolar affinity (Kᵢ=2 nM), was evaluated and compared to lifetime mode [14]. Ten different concentrations of thapsigargin were dispensed across a 384-well plate (0.8, 1, 2, 3, 4, 5, 6, 8, 10, and 50 nM thapsigargin concentrations, n=32 wells for each concentration). Matching DMSO controls (0.5 μL DMSO/50 μL total well volume) were located in the outer columns (1, 2, 23, and 24) of the 384 well plate. The spectra were decomposed using a four-component model (Eq. 9). The 2CS FRET change, acquired in spectral mode, is reflected by a decrease in the RFP-region (550-650 nm) of the fitted spectrum (FIG. 8A).

For direct comparison with spectral recording, nanosecond time-resolved fluorescence decay waveforms were acquired on the same samples (FIG. 8B). The fluorescence lifetime for each well was determined as described above (Eq. 7). A saturating concentration of thapsigargin (Tg), (50 nM) induced a 200 picosecond increase in the GFP lifetime, corresponding to a 6% decrease in FRET efficiency, as determined from the lifetime change compared to the donor-only control (Eq. 8), as shown in FIG. 8B. Heatmaps of FRET efficiency demonstrate excellent uniformity at each concentration across the plate (FIG. 8C, D), with greater precision evident for the spectral data.

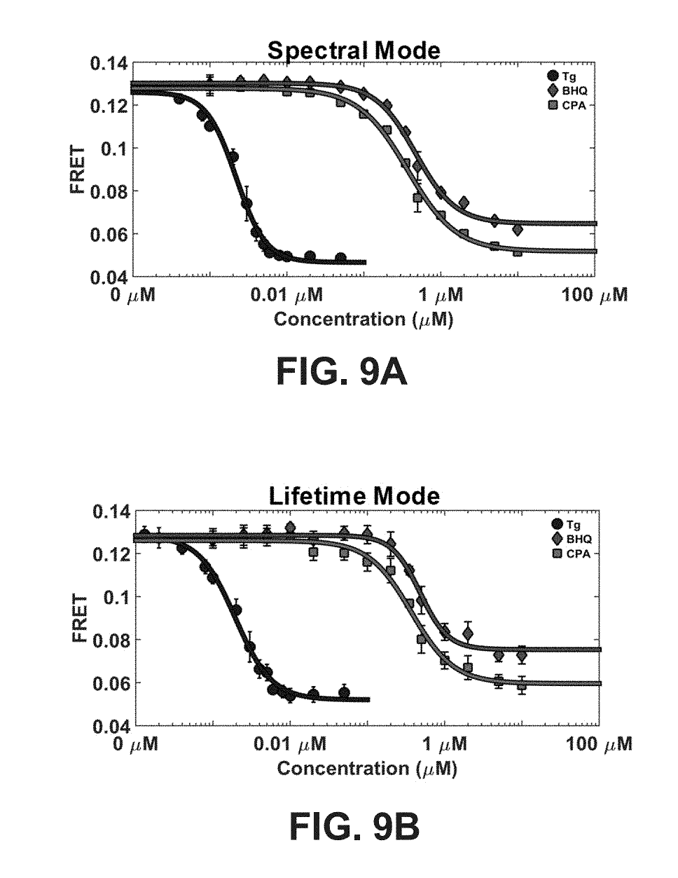

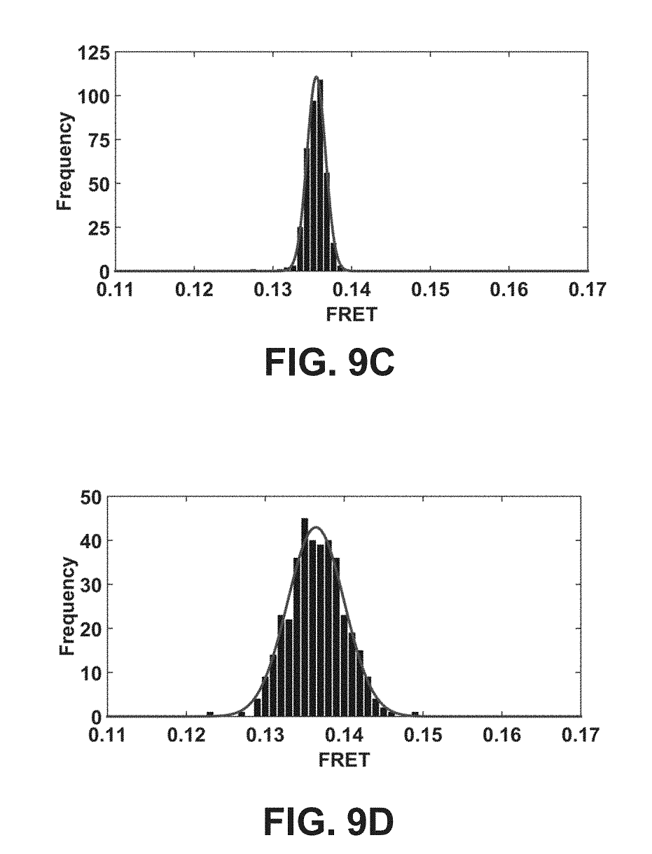

The sensitivity of FRET detection for both lifetime and spectral modes was further investigated by generating 14-point concentration-response curves for addition of thapsigargin, and two other well-established SERCA inhibitors 1,4-dihydroxy-2,5-di-tert-butylbenzene (BHQ) and cylcopiazonic acid (CPA). Reported Kᵢ's fall in the range of 2-7 μM for BHQ and 90-2500 nM for CPA. The concentration-dependent FRET change [14, 15], in response to the three known SERCA effectors, again exhibited excellent agreement between the FRET efficiency acquired from both spectral and lifetime modes (FIGS. 9C and D).

The experimentally determined apparent FRET equilibrium constants of thapsigargin and CPA agree with the previously reported Kᵢ values (FIGS. 9A and B). Note that error bars (n=8 wells) represent one standard deviation, not standard error of the mean. A tenfold difference from the FRET EC₅₀ and reported Kᵢ from functional activity assays was found for BHQ. This finding could reflect a difference between live-cell assays and biochemical functional assays performed on purified proteins. However, we have found the Kᵢ of BHQ to be 400 nM, as assessed by measuring the rate of ATP hydrolysis (data not shown).

The precision of the spectral unmixing and lifetime methods was further assessed by evaluating the FRET efficiency determined from 384 wells of 2CS cells, without the addition of drug. Histograms for all 384 wells of the 2CS cells-only control plate are shown in (FIGS. 9C and D). The average FRET efficiency was 13.7% (spectral mode) and 13.6% (lifetime mode) with standard deviations of 0.11% (spectral mode) and 0.36% (lifetime mode).

### Spectral Fitting Results in High Assay Precision, Even if Cellular Autofluorescence is High

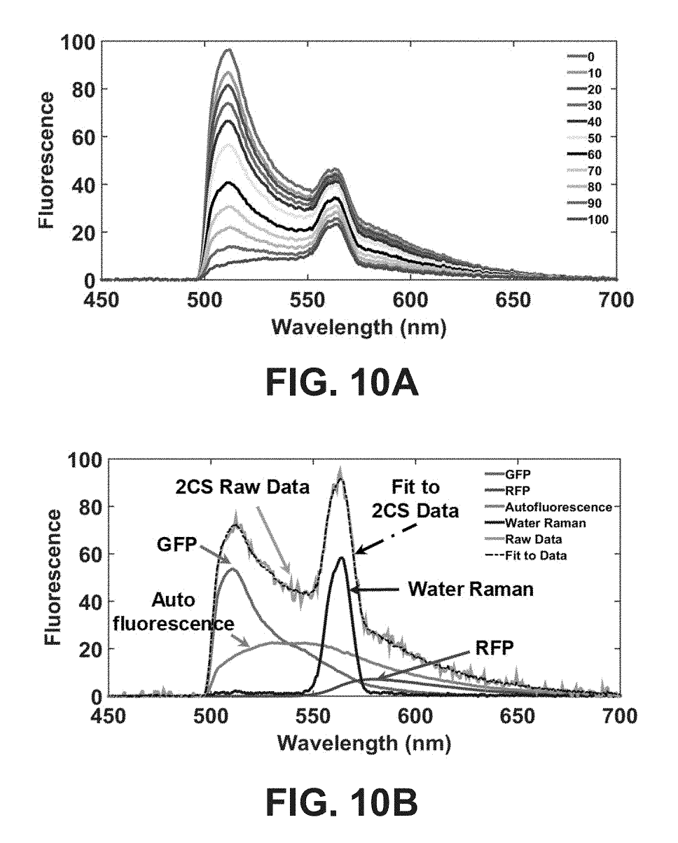

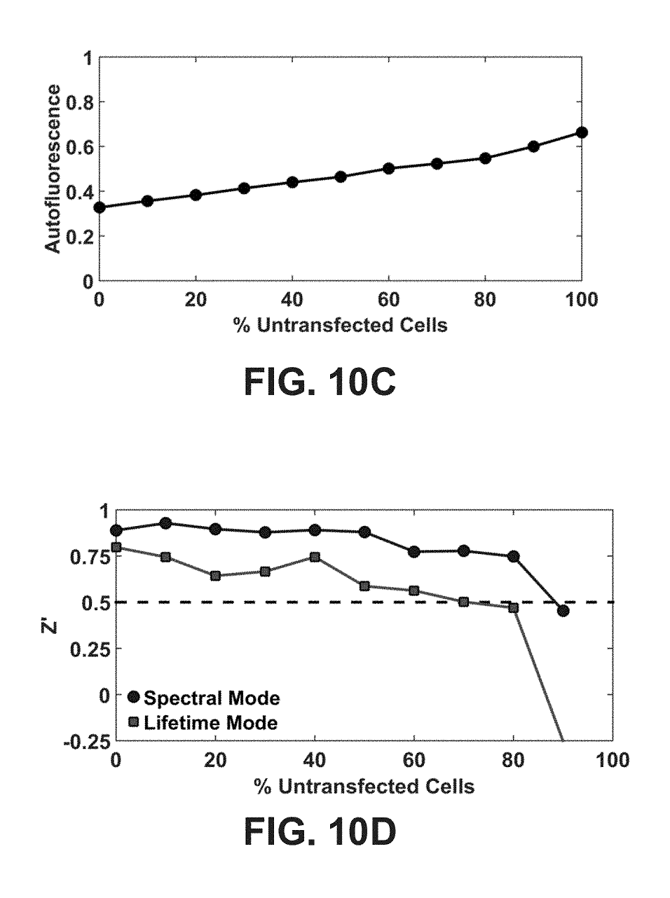

Live-cell fluorescence assays are prone to artifacts from cellular autofluorescence, dispensing error, and other variability in sample preparation [16]. Spectral unmixing is a very effective way to resolve the cellular autofluorescence component, maintaining high precision and accuracy of FRET determination. To simulate increasing autofluorescence (e.g., due to low biosensor expression or transient transfection), cells expressing the 2CS FRET biosensor were mixed with known amounts of untransfected cells, and dispensed across a column-wise gradient of one 384 well plate (FIG. 10A). The well volume (50 μL) and number of cells per well (50,000) were held constant. Spectra were analyzed as in FIG. 6D, as illustrated in FIG. 10B for 80% untransfected cells, resulting in much greater autofluorescence than that in FIG. 6D. The autofluorescence signal from each mixture was assessed by determining weighting coefficient c from (Eq. 9), and the expected linear increase was observed (FIG. 10C). To assess the effect of autofluorescence on the quality of the HTS assay, a 384-well plate was prepared with half the wells containing 100 nM thapsigargin and half being DMSO control wells (% v/v). These positive and negative controls were used to define the signal window for determination of assay quality factor Z′ [10, 17], yielding values of 0.90 (spectral mode) and 0.77 (lifetime mode), indicating that both modes provide an excellent assay for HTS (Z′>0.5), until the sample is diluted by 80% (lifetime) or 90% (spectral) with untransfected cells (FIG. 10D).

Accurate FRET Efficiency Determination from Cyan and Yellow Fluorescent Proteins

Although GFP and RFP (and other red-shifted FRET pairs) are less susceptible to compound fluorescence artifacts [18], the overwhelming majority of genetically-encoded FRET-based biosensors established and studied to date, involve cyan (CFP) and yellow (YFP) fluorescent proteins [19][129][129]. Accordingly, we present an illustration of the spectral plate reader's performance using this FRET pair.

Reference standards consisting of mCerulean (CFP) and mVenus (YFP) tethered by flexible linkers of increasing lengths of 5, 17, and 32 amino acids (designated C5V, C17V, and C32V, respectively) [20] have been widely used in FRET calibrations [20](Koushik et al. 2006). Various means to record the FRET signal, including subsequent lifetime and spectral analysis, have been previously applied. These controls can be used to calibrate and validate new FRET detection technology. The consensus FRET efficiencies for these constructs are 43±2 (C5V), 38±3 (C17V), and 31±2 (C32V) %. Transient transfections of HEK293 cells with these FRET reference standards and the appropriate donor CFP (mCerulean) and acceptor YFP (mVenus) labeled constructs were performed. The cells were harvested and assessed on the plate reader with excitation at 434/17 nm from a laser-driven light source (Energetiq). FRET efficiency was evaluated. Optimized transfection protocols for large-scale transient transfections, were found to obtain sufficiently high expression of the FRET standard constructs, so that contributions from autofluorescence and water Raman were negligible. Subsequently, only a two-component fit was required, as shown by the fitted and observed data with component spectra (FIG. 11A). The CFP/YFP ratio (FR in (Eq. 10) (Eq. 11) was converted to FRET efficiency as described in Supplemental Material (Derivation), using reported values of extinction coefficients and quantum yields [21]. The acceptor/donor fluorescence ratio, FR in (Eq. 11) was then calibrated to the previously reported FRET efficiency of C5V [20].

FRET efficiencies determined from spectral mode were in excellent agreement with the reported results 42.3±0.7 (C5V), 38.3±0.7 (C17V), and 29.0±0.6% (C32V) as shown in FIG. 11B. The precision was high; each bar represents the data from a single well. These results validate the capability of accurate FRET efficiency determination from CFP and YFP FRET pairs using the spectral unmixing method.

Investigation of a CFP and YFP FRET pair was evaluated with the well-known cameleon calcium sensor, in which the calmodulin-binding (M13) domain of myosin light chain kinase (MLCK) and calcium-binding domain of calmodulin are fused together, and located between two fluorescence proteins (eCFP/mCitrine). These calcium sensors have allowed cellular [Ca²⁺] to be monitored more directly and reliably than using calcium-sensitive dyes [22]. The endoplasmic reticulum-targeted cameleon sensor (D1ER) was selected because it is known to be sensitive to the SERCA inhibitors thapsigargin, CPA, and BHQ. Instead of detecting direct ligand binding, as in the 2CS studies reported above, the D1ER sensor can be used to monitor changes in ER calcium level [23]. Upon SERCA inhibition, calcium is no longer pumped into the ER, leading to calcium depletion through various mechanisms including IP3-gated receptors and ryanodine receptors. D1ER senses these changes as calcium binding leads to a conformational rearrangement of the biosensors, increasing FRET between CFP and YFP. Therefore, calcium depletion due to SERCA inhibition is detected as a decrease in FRET.

A HEK293 stable clone with constitutive expression of the D1ER cameleon calcium sensor was generated using G418 antibiotic selection. The localization of D1ER to the endoplasmic reticulum (ER) lumen was verified by microscopy, and expression remained constant over months in culture. Cells were harvested and dispensed into 384-well plates containing the same concentrations of thapsigargin, CPA, and BHQ, as previously evaluated with 2CS (FIG. 9). D1ER FRET was monitored only in spectral mode, because the CFP donor cannot be excited effectively by the 473 nm laser used in lifetime mode. ER calcium was monitored by repeatedly scanning the 384 well plate at three-minute intervals over a 120-minute period. The expected concentration and time-dependent decreases in FRET were observed. The high-affinity and selective SERCA inhibitor thapsigargin produced a sigmoidal FRET response with an equilibrium constant of 1.97 nM (ER calcium depletion shown at 120 min time point) (FIG. 11C).

The submicromolar SERCA inhibitors BHQ and CPA also produced a concentration-dependent effect. FRET EC₅₀ values were evaluated for each concentration curve, and agree with the expected values based on their affinity for SERCA. The time-dependent effect of SERCA inhibitors on endoplasmic reticulum (ER) calcium concentration can be determined by evaluating the rate of calcium depletion (FRET decrease) in response to titration with the inhibitors. Thapsigargin (Tg) irreversibly binds SERCA with high-affinity, and depletes calcium at a faster rate than BHQ and CPA, which have micromolar binding affinities (FIG. 11D). The time dependence of calcium depletion was evaluated by fitting each curve to the Hill equation to determine the rate of calcium depletion T₅₀. The T₅₀ of thapsigargin, BHQ, and CPA were 9.5, 17.4, and 11.7 minutes; respectively.

### Discussion

Accurately recording a fluorescence signal is an essential element of fluorescence spectrometers, microplate readers, fluorescence microscopes, flow cytometers, qPCR machines, chromatography, capillary array electrophoresis detectors, gel scanners, and sequencers. However, scanning a wavelength-selective filter (monochromator) through a range of emission wavelengths is usually employed only for research-grade fluorescence spectrometers, where data quality is more important than measurement speed. Microplate readers equipped with monochromators, now commonly available, could serve as alternatives to research-grade spectrometers. The primary difference between the two classes of instruments lies in the sample holder format and optical geometry, i.e., cuvettes and right angle for spectrometers, plastic plates and epi-illumination for plate readers. It is plausible to assume that a research-grade, cuvette-based fluorescence spectrometer will always provide much higher data quality than could ever be possible in a microplate reader, because the right-angle geometry, large sample volumes, and high-quality optics of the sample container minimize artifacts from light scattering and other sources of interfering background [24]. However until now, a comprehensive study that directly compares the data quality obtained with a fluorescence microplate reader to that produced by a fluorescence spectrometer did not exist (FIGS. 12A-C). The de facto standard approach to performance characterization of fluorescence spectrometers is the water Raman test [25]. The corresponding de facto standard approach to performance characterization of microplate readers is a fluorescence limit-of-detection test [26]. The data presented here strongly suggest that the quality gap between fluorescence spectrometers and microplate readers is much smaller than is generally assumed.

Genetically encoded FRET sensors are usually studied via imaging in a fluorescence microscope, using a rigorous 3-cube technique [1]. Corrections for cross-talk or bleed-through between the donor and acceptor excitation/emission [26] are determined by assessing images of fluorescence proteins expressed individually at each wavelength of interest. Acquisition at multiple excitation and emission wavelengths is typically required during each microscopy experiment. A major advantage revealed by these studies is that the shape of the full emission spectrum (2048 data points spread across the 300-800 nm wavelength region) can be used to reliably and accurately quantitate fluorescence signal without the need for exhaustive controls on each of day of experiments. Reference spectrum from control cell lines (in suspension) need only to be acquired one time. They were used to calibrate for detector sensitivity, and found to produce reproducible results for months on end. This drastically reduced the workload of maintaining and culturing control cell lines, thereby increasing productivity by reducing the number of conditions required for each experiment.

Another source of potential error in imaging experiments is autofluorescence contributions or interfering background emission. Microscopy methods rely on defining regions of interest, designed to isolate the cells of interest (e.g., cells that have the highest fluorescence signal) from the background. These results are averaged over a statistically meaningful number of cells [27]. The laborious nature of fluorescence emission correction from cellular autofluorescence and fluorescent protein crosstalk has made its usefulness for high-throughput drug screening impractical.

The spectral unmixing method incorporates essentially the same filter cubes and epi-illumination geometry as fluorescence microscopy. However, no attempt is made to image individual cells, as a simple lens directs the excitation light into a microplate well, and collects the emitted fluorescence signal. This cells-in-wells approach treats the cells as a homogeneous solution. Excitation of less than 50,000 cells placed in suspension, yielded enough photons to obtain high-quality spectra and fluorescence decay rates (lifetime) in a fraction of a second.

Spectral unmixing in fluorescence microscopy has previously been limited to low-throughput high-end instruments, typically based on a multi-anode PMT with 32 channels on roughly 10-nm spacing [28]. The present approach utilizes a linear array detector, and records at intervals of approximately 0.5 nm. This is substantially narrower than the width of the spectral features of interest, but advantageous nonetheless because oversampling improves the robustness of the fitting.

Many methods have been developed to decompose the fluorescence spectrum for the purposes of correcting waveform distortions associated with monochromator-based excitation and emission [27, 29]. The complexities of these methods are unsuitable for large-scale drug discovery campaigns. The development of a simplified method for multi-component spectral analysis for the determination of FRET efficiency from a live-cell biosensor, resulted in a assay suitable for high-throughput screening, as shown by the Z′ in FIG. 11D. Reference spectra of the known components are used to decompose the spectrum from the sample of interest (FIG. 6C). The spectral recording technology allows for robust measurements, with inexpensive equipment in comparison to fluorescence microscopes and flow cytometry. The spectral unmixing method can also be used to solve for unknown components such as sample contamination or, as shown in our following companion paper [30], the direct identification of fluorescent compounds (false-positives) during a high-throughput drug screen.

Data acquired by both spectral and lifetime modes were shown to be extremely precise. The focus of this article is to demonstrate the novel spectral recording technology, as the lifetime technology has previously been evaluated [3, 5, 6]. However, this is the first demonstration of the top-read fluorescence lifetime plate reader. This optical configuration is advantageous, as inexpensive black-bottom microplates can be used, instead of glass-bottom microplates. Another benefit is that temperature can be controlled by placing a heat source underneath the microplate. These studies directly compared two novel fluorescence plate reader technologies. A three-fold increase in precision was found for spectral mode over lifetime mode as shown in FIGS. 9C and 9D. These studies utilized data analysis methods that can currently be used for large-scale drug discovery efforts, as they are not computationally taxing, and can be performed in real time. The precision of the lifetime method is likely to be increased by development of more sophisticated analysis methods. For example, in these studies a single-exponential model was used to fit the lifetime data, and no attempt was made to correct the lifetime data for artifacts from cellular autofluorescence. Demonstrations of advancements in using more-rigorous global lifetime analysis can be found in the accompanying article, reporting high-throughput screening performance [30].

The increased precision from spectral mode, as compared with lifetime mode, is likely to be observed primarily when employing two-color biosensors, in which every donor has an acceptor on the same molecule. In biosensors created by reacting dyes with amino acid side chains, and/or involving donor and acceptor on different proteins, lifetime detection has the advantage of resolving heterogeneous populations of donors. The unique structural resolution of the lifetime mode also permits the resolution of multiple structural states of the biosensor [1, 31, 32], providing more detailed structural information about the results of screening.

Two distinct classes of live-cell FRET biosensors were shown, those that are designed specifically for structure determination (FIG. 8A; FIG. 9A; FIG. 11B), and those that exploit a structural change in the biosensor for the purpose of quantitating the concentration of some species, such as [Ca²⁺], in the cellular milieu (FIGS. 11C and 11D). There are many applications in which a better way to detect structural changes of either a cytosolic or membrane protein via FRET would be valuable. Developing and engineering fluorescent protein FRET biosensors can be challenging, as they can be difficult to express (low signal, high cellular autofluorescence), they exhibit a low FRET signal (because the donor and acceptor are too far apart), or the dynamic range of the signal window may be limited. These problems can be overcome, as incredibly small changes in FRET signal can be detected using this spectral and lifetime detection technology. Even more so, detection of weak GFP fluorescence masked by cellular autofluorescence, was clearly resolved using the spectral unmixing methods.

Beyond increased sensitivity and precision, the rapid acquisition rates reported here (10 wells or more per sec) allow for time-course studies. Previous reports suggest that the resolution and sensitivity of microplate readers is inadequate to directly monitor calcium flux in live-cells [33]. To date, high-throughput screening with cameleon sensors, such as D1ER, has been achieved using laborious high-content imaging [34], or calcium-sensitive fluorescent dyes with CCD-camera based plate readers (FLIPR, Molecular Devices). To our knowledge, this is the first time changes in live-cell calcium levels were shown to be monitored with high speed and precision in a fluorescence microplate reader using a FRET based biosensor (FIG. 11C). Short acquisition times of 200 ms per well were used to repeatedly scan portions of high-density microplates, and monitor calcium flux at rates, comparable to standard microscopy techniques; except across a range of chemical perturbations (FIG. 11D) Thus, the D1ER FRET-based calcium biosensor may be a powerful tool for high-throughput screening, linking the structural perturbations of 2CS to functional changes, at the level of calcium dysregulation in live-cells.

The spectral and lifetime detection methods presented here are widely applicable across the life sciences, beyond high-throughput and high-content assays. Potential assays, which may benefit from this platform are phenotypic screening of libraries of mutant constructs, recombinant antibody development, or protein stability assays, where sample quantities are limited. Future technological developments may also include cell sorting by the addition of microfluidics devices.

In conclusion, a new instrument records fluorescence spectra in a microplate reader, at speeds fully compatible with high-throughput screening applications. The resolution of the recorded spectra is improved in comparison to that provided by standard cuvette-based fluorescence spectrometers, even though the acquisition rates are 100 times faster and sample volumes 100 times smaller (FIGS. 12A-C). The novel spectral and lifetime technologies were thoroughly evaluated, and when used together are complementary, creating a new combination of precision and resolution, particularly in applications to living cells expressing genetically encoded FRET biosensors. Accuracy and precision is comparable to or greater than those achieved with much lower throughput instruments, such as cuvette-based spectrofluorometers and fluorescence microscopes. These technical breakthroughs in fluorescence recording enable their use in high-throughput screening applications, as illustrated in the following article [30].

### Supplementary Material

### Reference Spectra Determination for Fluorescence Emission Components of 2CS FRET Biosensor

The spectral unmixing process yields a decomposition of the observed (experimental) spectrum into a linear combination of biochemically meaningful component spectra. In the case of the experiments reported here, a minimum of four components (basis spectra) must be included in the spectral fitting: donor fluorescence (GFP), acceptor fluorescence (RFP), cellular autofluorescence, and Raman scattering of the buffer (effectively the inelastic Raman scattering of water). Scattered exciting light, stray light, background fluorescence of the plate material, and impurities are other possibilities. The quality of the fit is typically judged by the residuals (errors) between the observed and fitted spectrum. A challenge that was overcome was that it is not possible to prepare pure samples of the individual components except in the case of the water Raman.

### Water Raman:

A signal-to-noise comparison of the inelastic light scattering due to the water Raman band after excitation at 473 nm (GFP excitation) was performed. Comparison water Raman spectra were acquired on three commercially-available fluorescence microplate readers equipped with emission monochromators. The acquisition rate in these experiments (FIG. 12A) was set at one second per wavelength, and the wavelength spacing at 1 nm. A water Raman spectrum (black) was also acquired on a cuvette-based fluorescence spectrometer (Cary Eclipse, xenon flash-lamp excitation) with the same acquisition time and wavelength interval (FIG. 12B). FIG. 12C shows the superposition of six spectra, each corresponding to a different well of a 384 plate, at 200 ms acquisition time per well, acquired with the spectral unmixing plate reader (Fluorescence Innovations). The spectra have been normalized to the same area under the curve to better illustrate the very high repeatability. The relatively narrow water Raman spectra acquired with the spectral unmixing plate reader, allows for the capability to extract spectral features, and produce robust and high precision measurements. Note, too, the very low background signal from the black polypropylene plates on either side of the water Raman band, despite the epi-illumination geometry.

### Cellular Autofluorescence

Excitation of living cells in the 450-500 nm wavelength range produces a background autofluorescence signal, generally attributed to flavins [35]. The fluorescence spectrum of 50 μL of untransfected HEK293 cells suspended in PBS at a concentration of 50,000 cells per well of a 384 well plate is shown in FIG. 13, the sharp and narrow peak of the water Raman feature at 560 nm was used to subtract the water Raman signal from cellular autofluorescence spectrum, thereby producing the autofluorescence reference spectrum.

GFP reference spectrum. The reference spectra of GFP (eGFP) were acquired from purified fluorescent protein preparations, and by subtracting each component spectrum (water Raman, autofluorescence, and the signal from fluorescent protein) using live-cells expressing the fluorescent proteins. The former approach avoids the need to consider the autofluorescence contribution, but the signal of the green or red fluorescent proteins may be dependent on buffer conditions, and vary with environment surrounding the purified protein.

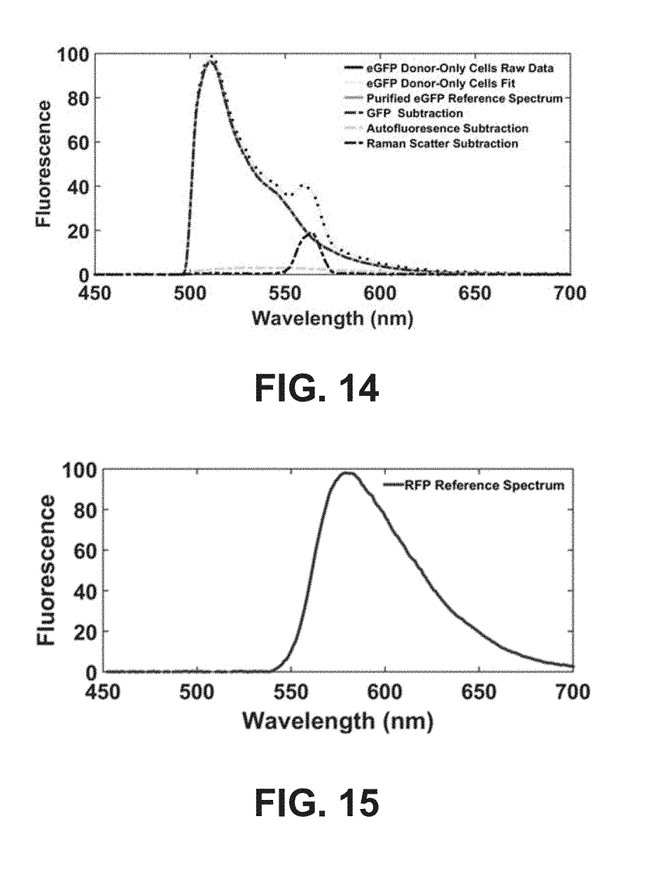

The reference spectrum of GFP, the donor fluorescent protein used for the red/green FRET pair of the two-color SERCA biosensor, was acquired from HEK293 cells overexpressing high levels of an appropriately donor-labeled control cell line. This cell line expressed a genetically-encoded construct, where GFP was fused to an intra-sequence flexible loop on SERCA2a's cytosolic nucleotide-binding domain (at residue 509). The GFP reference spectrum was generated by subtracting the contribution of the cellular autofluorescence and water Raman from the observed GFP SERCA control cell line (FIG. 14). The fluorescence signal of the GFP-SERCA fusion protein was much higher than the background signal from the cells themselves (cellular autofluorescence). Therefore, a very subtle subtraction of the autofluorescence signal was made for this particular cell line. However, the shift in the shape of the subtracted GFP reference fluorescence spectrum did not significantly alter the results of the spectral unmixing methods. This reference spectrum was used to accurately resolve the fluorescence signal of GFP from a cell line over-expressing the two-color SERCA FRET biosensor. Further comparison of the inferred GFP reference spectrum from live-cells with the spectrum of purified protein is shown in FIG. 14, by the overlay of GFP subtraction and the denoted solved GFP reference spectrum. Although small variations in the overall shape of the reference GFP spectrum acquired from live-cells and purified protein preparations have been previously reported [35], these differences did not distort the results obtained from the spectral unmixing methods.

RFP reference spectrum. The reference spectrum of the red fluorescent protein (tagRFP) was acquired using 532 nm laser excitation, which eliminated the contribution from cellular autofluorescence. Excitation at a longer wavelength also shifts the water Raman band to the 650 nm wavelength. The effect of the water Raman band is much weaker because of its λ⁴ dependence. Finally, the RFP-SERCA fluorescent fusion construct had much higher expression than the intrasequence labeled GFP-SERCA control construct. The RFP reference spectrum is shown in FIG. 15.

The four reference spectra shown in FIG. 6A were used for all studies involving spectral unmixing of the 2CS construct with GFP and RFP-labeled fluorescent proteins.

### Derivation of the Spectral FRET Equation for 2-Color Biosensor

### Nomenclature:

ε_{A}: molar absorptivity of acceptor at λₑₓ

ε_{D}: molar absorptivity of donor at λₑₓ

Φ_{FRET}: Fluorescence quantum yield of FRET efficiency

Φ_{F,D}: Fluorescence quantum yield of donor, including potential change due to FRET

Φ_{F,A}: Fluorescence quantum yield of acceptor

### Assumptions:

1. # Donor excited states proportional to ε_{D}

2. # Acceptor excited states proportional to ε_{D} Φ_{FRET}+ε_{A}

3. # Donor emission events proportional to ε_{D} Φ_{F,D}

4. # Acceptor emission events proportional to (ε_{D} Φ_{FRET}+ε_{A})Φ_{F,A}

### Derivation:

Let FR be the FRET ratio of total number of photons emitted by acceptor to total number of photons emitted by donor (Eq. 11); assuming the CCD linear-array spectrograph detector has been corrected for wavelength dependence of its response. The total fluorescence emission F of each component's reference spectrum is dependent on the scalar coefficients shown as the sum of the linear combination of the four components of the fluorescence emission signal from as shown in (Eq. 9), where b represents the scalar coefficient for RFP (acceptor), and a the scalar coefficient for GFP (donor).

$$FR = \frac{Acceptor fluorescence}{Donor fluorescence} = \frac{{bF}_{REF}}{{aF}_{OFF}} . \qquad Eq . 12$$

Substitution of extinction coefficients (molar absorptivity) and quantum yields, which represent the total acceptor and donor fluorescence from the FRET biosensor gives following equation:

$$FR = \frac{( ɛ D Φ FRET + ɛ A ) Φ F , A}{ɛ D Φ F , D} . \qquad Supp Eq . 1$$

However, the contribution of FRET to the donor fluorescence when the rate of fluorescence energy transfer is zero, gives Φ_{F,D}=Φ_{F,D}(E=0)(1−Φ_{FRET}). Substitution of this equation for the donor quantum yield at (E=0) yields:

$$FRET = \frac{FR * Φ F , D / Φ F , A - \text{?} A / \text{?} D}{1 + FR * Φ F , D / Φ F , A} . \\ \text{?} \text{indicates text missing or illegible when filed} \qquad Supp Eq . 1$$

where QR (ΦF,D;ΦF,A) is the ratio of the donor fluorescence quantum yield under non-FRET conditions to the acceptor fluorescence quantum yield. AR (εA/εD) is the extinction coefficient ratio of acceptor molar absorptivity to the donor molar absorptivity at 473 nm wavelength excitation. Note that FR is the only experimentally derived quantity as the quantum yields and extinction coefficients of the eGFP and tagRFP have been previously reported, and were determined for 473 nm wavelength excitation [21].

The quantum yield ratio could be determined directly, although quantum yields are notoriously difficult to measure with very high accuracy. Moreover, the fluorescence ratio (FR) depends on the wavelength dependence of the CCD spectrograph detector. In other words, the measured FR needs to be corrected to a constant detector response (no wavelength dependence).

A β-factor was experimentally determined, and used to calibrate for the difference in the overall spectrograph sensitivity at longer wavelengths (wavelength-dependent response and the wavelength dependence of the grating's diffraction efficiency) The dispersion of the light by the grating is non-linear, so shorter wavelength light is spread out more than longer wavelength light. For simplicity the β-factor was designated to quantum yield factor (QR) yielding:

$$FRET = \frac{β ( QR ) * FR - ( \text{?} A / \text{?} D )}{1 + β ( QR ) * FR} . \\ \text{?} \text{indicates text missing or illegible when filed} \qquad Supp Eq . 2$$

The β value (correction factor) used to correct for difference in GFP and RFP detector sensitivity was determined using the fluorescence lifetime data of the τ_{D} (GFP-SERCA) and τ_{DA} (2CS) cell lines, and solving for the apparent FRET efficiency.
Solving for the FRET efficiency of 2CS (above) for β gives

β=[(τD/τDA)(1+εA/εD)−1]/FR Supp Eq. 4

where the τ values are fluorescence lifetimes acquired from the appropriate donor-only and donor-acceptor (FRET) cell lines.

FIG. 16 shows a single β-value (corrected QR=0.83) that gives excellent agreement between the FRET efficiency values calculated by lifetime and by spectra. The data used for this calibration of FRET efficiency was from the 12-point thapsigargin 2CS dose-response studies (FIGS. 9A and 9B). Each point on the plot shows the FRET efficiency obtained from lifetime mode (direct determination using (Eq. 2), and after solving for the β-factor using the spectral FRET efficiency equation (Eqs. 5 and 6) at 12 different concentration of Thapsigargin. For all FRET pairs, each correction factor was obtained by using reported values of the fluorescent protein quantum yield and molar absorptivity [21], and correcting for CCD detector sensitivity. The β-factors for the CFP/YFP variants were not determined by direct comparison to fluorescence lifetime measurement, because a microchip pulsed laser with the appropriate excitation wavelength does not exist. The β-factor for the quantum yield ratio of the mCerulean and mVenus FRET pair was determined by using the known FRET efficiency of the C32V construct [20], and solving for the β-factor to match the expected value. This β-factor was then used to determine the FRET efficiency of the C17V and C5V construct as shown in FIG. 11B. The beta-factor for the eCFP/mcitrine FRET pair was determined by estimating the FRET efficiency of the D1ER FRET biosensor, under normal cellular conditions, using previously reported fluorescence lifetime measurements [36]. QR in supplemental table is the β-factor corrected quantum yield ratio from each FRET pair.

TABLE 1
Quantum Yield and Molar Absorptivity Ratios
for Donor and Acceptor FRET Pairs

| FRET pair | QR | AR |
|---|---|---|
| eGFP and tagRFP (2CS) | 0.83 | 0.05 |
| eCFP and mCitrine (D1ER) | 0.46 | 0.12 |
| mCerulean and mVenus | 0.37 | 0.10 |

### Multiple-Exponential Lifetime Fitting

The donor-only control cell line, where SERCA2a is fused to GFP, was used to determine the FRET efficiency using E=1−τ_{DA}/τ_{D}. The fluorescence waveform was fit using both one- and two-exponential model as shown in FIGS. 17A and 17B. The χ² of each fit across the nanosecond domain was determined from the sum of the residuals obtained from the fit. The χ² was reduced for the two-exponential model and this model was used for subsequent global lifetime analysis to determine distance distributions. Adding more than two exponentials to the model was not found to increase the goodness of the fits.
1. Hou, Z., et al., Two-color calcium pump reveals closure of the cytoplasmic headpiece with calcium binding. PLoS One, 2012: p. accepted.
2. Pallikkuth, S., et al., Phosphorylated Phospholamban Stabilizes a Compact Conformation of the Cardiac Calcium ATPase. Biophys J, 2013. 105(8): p. 1812-21.
3. Gruber, S. J., et al., Discovery of enzyme modulators via high-throughput time-resolved FRET in living cells. J Biomol Screen, 2014. 19(2): p. 215-22.
4. Shcherbo, D., et al., Practical and reliable FRET/FLIM pair of fluorescent proteins. BMC biotechnology, 2009. 9: p. 24.
5. Petersen, K. J., et al., Fluorescence lifetime plate reader: resolution and precision meet high-throughput. Rev Sci Instrum, 2014. 85(11): p. 113101.
6. Tram, S. H., et al., ATP Binding Cassette Transporter Structure Changes Detected by Intramolecular Fluorescence Energy Transfer for High-Throughput Screening. Molecular pharmacology, 2015. 88(1): p. 84-94.
7. Muretta, J. M., et al., High-performance time-resolved fluorescence by direct waveform recording. Rev Sci Instrum, 2010. 81(10): p. 103101.
8. Guzman, C., C. Oetken-Lindholm, and D. Abankwa, Automated High-Throughput Fluorescence Lifetime Imaging Microscopy to Detect Protein-Protein Interactions. Journal of laboratory automation, 2015.
9. Suzuki, M., et al., A high-throughput direct fluorescence resonance energy transfer-based assay for analyzing apoptotic proteases using flow cytometry and fluorescence lifetime measurements. Analytical biochemistry, 2015. 491: p. 10-7.
10. Cornea, R. L., et al., High-throughput FRET assay yields allosteric SERCA activators. J Biomol Screen, 2013. 18(1): p. 97-107.
11. Suhling, K., et al., Imaging the environment of green fluorescent protein. Biophys J, 2002. 83(6): p. 3589-95.
12. Leavesley, S. J., et al., Assessing FRET using spectral techniques. Cytometry. Part A: the journal of the International Society for Analytical Cytology, 2013. 83(10): p. 898-912.
13. Thorn, T. L. a. K. Fluorescent protein properties. 2015; Available from: http://nic.ucsf.edu/FPvisualization/.
14. Wootton, L. L. and F. Michelangeli, The effects of the phenylalanine 256 to valine mutation on the sensitivity of sarcoplasmic/endoplasmic reticulum Ca2+ ATPase (SERCA) Ca2+ pump isoforms 1, 2, and 3 to thapsigargin and other inhibitors. The Journal of biological chemistry, 2006. 281(11): p. 6970-6.
15. Michelangeli, F. and J. M. East, A diversity of SERCA Ca2+ pump inhibitors. Biochemical Society transactions, 2011. 39(3): p. 789-97.
16. Palmer, A. E., et al., Design and application of genetically encoded biosensors. Trends in biotechnology, 2011. 29(3): p. 144-52.
17. Zhang, J. H., T. D. Chung, and K. R. Oldenburg, A Simple Statistical Parameter for Use in Evaluation and Validation of High Throughput Screening Assays. J Biomol Screen, 1999. 4(2): p. 67-73.
18. Lam, A. J., et al., Improving FRET dynamic range with bright green and red fluorescent proteins. Nature methods, 2012. 9(10): p. 1005-12.
19. Addgene. Fluorescent Protein Guide: Biosensors. 2015; The nonprofit plasmid repository]. Available on the world wide web at addgene.org/fluorescent-proteins/biosensors.
20. Koushik, S. V., et al., Cerulean, Venus, and VenusY67C FRET reference standards. Biophysical Journal, 2006. 91(12): p. L99-L101.
21. Cranfill, P. J., et al., Quantitative assessment of fluorescent proteins. Nature methods, 2016. 13(7): p. 557-62.
22. Palmer, A. E. and R. Y. Tsien, Measuring calcium signaling using genetically targetable fluorescent indicators. Nature protocols, 2006. 1(3): p. 1057-65.
23. Palmer, A. E., et al., Bcl-2-mediated alterations in endoplasmic reticulum Ca2+ analyzed with an improved genetically encoded fluorescent sensor. Proceedings of the National Academy of Sciences of the United States of America, 2004. 101(50): p. 17404-9.
24. Lakowicz, J., Principles of Fluorescence Spectroscopy, 3rd ed. Springer 2006.
25. Scientific, H., Signal to Noise Ratio Explained. 2015.
26. Thomsen, V., D. Schatzlein, and D. Mercuro, Limits of detection in spectroscopy. Spectroscopy, 2003. 18(12): p. 112-114.
27. Zhang, L., et al., Spectral wide-field microscopic fluorescence resonance energy transfer imaging in live cells. Journal of biomedical optics, 2015. 20(8): p. 86011.
28. Larson, J. M., The Nikon C1si combines high spectral resolution, high sensitivity, and high acquisition speed. Cytometry. Part A: the journal of the International Society for Analytical Cytology, 2006. 69(8): p. 825-34.
29. Mustafa, S., et al., Quantitative Forster resonance energy transfer efficiency measurements using simultaneous spectral unmixing of excitation and emission spectra. Journal of biomedical optics, 2013. 18(2): p. 26024.
30. Schaaf, T. M., et al., High-Throughput Spectral and Lifetime-Based FRET Screening in Living Cells to Identify Small-Molecule Effectors of SERCA. Journal of biomolecular screening, 2016.
31. Li, J., et al., Structural and functional dynamics of an integral membrane protein complex modulated by lipid headgroup charge. Journal of molecular biology, 2012. 418(5): p. 379-89.
32. Dong, X. and D. D. Thomas, Time-resolved FRET reveals the structural mechanism of SERCA-PLB regulation. Biochemical and biophysical research communications, 2014. b 449(2): p. 196-201.
33. Meijer, M., et al., Comparison of plate reader-based methods with fluorescence microscopy for measurements of intracellular calcium levels for the assessment of in vitro neurotoxicity. Neurotoxicology, 2014. 45: p. 31-7.
34. Honarnejad, K., et al., FRET-based calcium imaging: a tool for high-throughput/content phenotypic drug screening in Alzheimer disease. Journal of biomolecular screening, 2013. 18(10): p. 1309-20.
35. Ogikubo, S., et al., Intracellular pH sensing using autofluorescence lifetime microscopy. J Phys Chem B, 2011. 115(34): p. 10385-90.
36. Avezov, E., et al., Lifetime imaging of a fluorescent protein sensor reveals surprising stability of ER thiol redox. J Cell Biol, 2013. 201(2): p. 337-49.

### Example 2

### High-Throughput Spectral and Lifetime-Based FRET Screening in Living Cells to Identify Small-Molecule Effectors of SERCA

A robust high-throughput screening (HTS) strategy has been developed to discover small-molecule effectors targeting the sarco/endoplasmic reticulum calcium ATPase (SERCA), based on a fluorescence microplate reader that records both the nanosecond decay waveform (lifetime mode) and the complete emission spectrum (spectral mode), with high precision and speed. This spectral unmixing plate reader (SUPR) was used to screen libraries of small molecules with a fluorescence resonance energy transfer (FRET) biosensor expressed in living cells. Ligand binding was detected by FRET associated with structural rearrangements of green (GFP, donor) and red (RFP, acceptor) fluorescent proteins fused to the cardiac-specific SERCA2a isoform. The results demonstrate accurate quantitation of FRET along with high precision of hit identification. Fluorescence lifetime analysis resolved SERCA's distinct structural states, providing a method to classify small-molecule chemotypes on the basis of their structural effect on the target. The spectral analysis was also applied to flag interference by fluorescent compounds. FRET hits were further evaluated for functional effects on SERCA's ATPase activity via both a coupled-enzyme assay and a FRET-based calcium sensor. Concentration-response curves indicated excellent correlation between FRET and function. These complementary spectral and lifetime FRET detection methods offer an attractive combination of precision, speed, and resolution for HTS. This Example is also available as Schaaf et al., SLAS Discov. 2016. Mar. 22(3): 262-273. [Note: The journal was published from 1996 through 2016 with the title Journal of Biomolecular Screening. Its name changed in 2017 to SLAS Discov. The older citation prior to this change includes: Schaaf et al., J Biomol Screen. 2016 Nov. 29. pii: 1087057116680151.]

### Introduction

The preceding article reports the performance of a novel microplate-reader that records fluorescence emission spectra with an unprecedented combination of speed and precision. That study indicated that this spectral-unmixing plate-reader (SUPR), when combined with a previously described fluorescence lifetime plate-reader (FLTPR) that achieves a similarly high level of performance using nanosecond time resolution, is ready for HTS. This study demonstrates an initial application.

The specific target in this work is sarco/endoplasmic reticulum calcium ATPase (SERCA) [1, 2]. which has therapeutic relevance for a wide range of diseases, including heart failure [2], multidrug-resistant leukemia [3], and type II diabetes [4]. SERCA is a critical enzyme, as it maintains calcium homeostasis by actively pumping calcium from the cytosol into the endoplasmic or sarcoplasmic reticulum. Over a dozen human SERCA isoforms have been described, each with tissue-specific expression and distinct structural and functional characteristics. Specialized SERCA isoforms are predominantly found in electrically-excitable cells, such as myocytes and cardiomyocytes, where calcium-cycling is necessary for the contractile apparatus to function properly [5].

Recently, SERCA-based therapy based on calcium up-regulation by percutaneous administration of gene therapy was tested in cardiac disease clinical trials (CUPID study). SERCA overexpression by administration of adeno-associated virus (AAV), delivered directly to the hearts of patients experiencing end-stage heart failure, was shown to correct deficits in SERCA2a (cardiac-isoform) expression and activity, known to be correlated to impairment in cardiac (diastolic) function [6]. Despite encouraging early results, SERCA AAV gene therapy failed to meet primary end goals in phase IIb clinical trials. This failure was attributed to the limitations of AAV gene therapy, including the development of neutralizing antibodies [7], and difficulties of maintaining constant, long-term expression of the large 110 kD enzyme. We continue to explore alternative SERCA-based gene therapy strategies [8-10] but are also actively pursuing the search for small-molecule SERCA effectors capable of ameliorating the SERCA malfunction found in numerous degenerative diseases [5].

Initial screening campaigns evaluated structural perturbations using a reconstituted membrane system and fluorescence resonance energy transfer (FRET) detection between SERCA and its regulatory partner phospholamban (PLB). In these studies, conventional fluorescence emission spectral recording was utilized for large-scale screening, and resulted in the discovery of small-molecule activators of SERCA [1]. We continued our development of SERCA biosensors by engineering a genetically-encoded intramolecular FRET sensor; donor and acceptor fluorescent proteins were fused to specific locations on SERCA's cytoplasmic headpiece, known to undergo large-scale, physiologically-relevant, structural changes (5-10 nm), as depicted by the known crystal structures [11],[2], This type of assay lends itself naturally to a structure-based screening campaign, in which the results are not only related to variation of compound structure, but also to variation of specific structural changes in the labeled target.

This two-color SERCA (2CS) biosensor utilizes green and red fluorescent proteins as a FRET pair. These red-shifted fluorescent proteins are less sensitive to cell autofluorescence and fluorescent compound interference, compared with their blue-shifted CFP and YFP counterparts. The existence of multiple 2CS FRET populations was previously identified using single-molecule fluorescence lifetime microscopy [12]. These studies elucidated SERCA's sensitivity to PLB and calcium, revealing insights into residues involved in this structure-activity relationship [13]. This set the stage for a proof-of-principle structure-based small-molecule screen, using a prototype fluorescence lifetime (FLT) microplate reader [2],[14], This seminal study proved that a genetically-encoded FRET sensor could be stably expressed in human embryonic kidney (HEK293) cells and utilized for HTS in a microplate format.

The present work utilizes a novel top-read fluorescence lifetime plate reader, which involves an epi-illumination geometry, thereby allowing for temperature control and the use of inexpensive black-bottom 384- or 1536-well plates. The preceding article established the technical feasibility of decomposing the fluorescence emission spectra into a linear combination of component spectra, from which the FRET efficiency (FRET) can be calculated. That study demonstrated high screening quality (high Z′ value) and the ability to resolve minute FRET changes (0.5%), even when the cellular autofluorescence was artificially increased. This study involves screening a small-molecule library (National Clinical Collections 1 & 2), which contains a collection of compounds that have already been evaluated in pre-clinical and clinical trials. The ability to accurately determine changes in FRET from the 2CS biosensor from two independent fluorescence measurements (spectral and lifetime) increases the confidence of hit selection.

### Materials and Methods

### Cell Culture

HEK293 (originally derived from human embryonic kidney) cells were maintained in phenol red-free DMEM from Gibco (Waltham, Mass.) supplemented with 2 mM GlutaMAX (Gibco), 10% fetal bovine serum (FBS), from Atlanta Biologicals (Lawrenceville, Ga.), and 1 IU/mL penicillin/streptomycin (Gibco) and grown at 37° C. with 5% CO₂. HEK293 cell lines were used to generate stable clones overexpressing the FRET-based biosensors and corresponding donor and acceptor labeled control cell lines [2]. Three days prior to screening, the stable cell lines were expanded in five T225 flasks from Corning Inc. (Corning, N.Y.). On each day of screening and FRET hit retesting, approximately 300 million cells were harvested by treatment of Tryple from Invitrogen (Carlsbad, Calif.), washed three times in phosphate buffer solution (PBS) with no magnesium or calcium from Thermo Scientific (Waltham, Mass.) by centrifugation at 300 g, filtered using 70 μm cell strainers (Corning), and diluted to 10⁶ cells/mL using an automated countess cell counter (Invitrogen). On each day of screening, cell viability was assessed using the trypan blue assay.

After resuspension and dilution in PBS, the cells were constantly and gently stirred using a magnetic stir bar at room temperature, keeping the cells in suspension and evenly distributed to avoid clumping. During screening, cells were then dispensed into five 384 well assay plates, one containing no compound, one containing eight-point concentration curves of three known SERCA effectors, and three containing the NCC libraries 1 and 2. The same methods were applied for subsequent FRET testing of the reproducible hits identified in the pilot screen. Concentration-response curves (CRC) of the FRET hits were assessed at multiple time points by repeatedly scanning the 384-well plates. HEK293 stable clones expressing either the D1ER calcium FRET sensor (CFP/YFP) or the 2CS biosensor were used to evaluate the hits. The D1ER calcium sensor monitored changes in endoplasmic reticulum [Ca²⁺] [15, 16]. The methods and protocols for the D1ER cells were identical to those for 2CS, with the exception that they were evaluated only in spectral mode, and the 384-well plates containing compound CRCs were scanned every three minutes for two-hours.

### Liquid Dispensing

Cells were dispensed using a Multidrop Combi liquid dispenser from Thermo (Pittsburgh, Pa.), at a density of 10⁶ cells/mL. Compounds were diluted in DMSO and dispensed either using an automated Echo acoustic liquid dispenser from Labcyte (Sunnyvale, Calif.) or a Mosquito liquid dispenser from TTP Labtech (Melbourn, UK).

Cells and compound mixtures were dispensed into 384-well flat, black-bottom polypropylene plates from Greiner (Kremsmünste, Austria). The cells were dispensed at room temperature into plates containing test compounds. They were incubated with compound for 20, 60, 90, and 120 minutes, and then scanned in both lifetime and spectral modes. 727 compounds from the NCC 1 and 2 compound libraries were purchased from Evotec (Hamburg, Germany), formatted into 96-well mother plates using a Biomek FX liquid dispenser from Beckman Coulter (Brea, Calif.), and subsequently formatted across three 384-well plates at 50 nL (10 μM final concentration per well) using an Echo liquid dispenser from Labcyte. Control wells containing matching % v/v DMSO were formatted into unused wells and columns 1, 2, 23, and 24 of the assay plates. The eleven reproducible FRET hits were purchased from three different vendors Sequoia Sciences (Saint Louis, Mo.), Tocris (Minneapolis, Minn.), or Santa Cruz (Santa Cruz, Calif.) depending on their availability.

### Instrumentation and Data Analysis

An in-depth description of the fluorescence instrumentation is described in the previous article [17] and in the supplemental material. For lifetime mode, the observed fluorescence waveform was convolved with the instrument response function, and the average energy transfer efficiency (E=1−τ_{DA}/τ_{D}) was calculated from the average lifetimes of donor τ_{D} and donor-acceptor, τ_{DA}, FRET cell lines. The structural correlates for FRET were modeled as previously described [18-20], assessing the nanosecond time dependence of the TR-FRET waveforms according to (Eq.)1 (Eq. 6) (previously described above):

$$\begin{matrix} {F}_{D} ( t ) = \text{?} {A}_{c} exp ( - 1 / \text{?} ) & ( Eq . 1 ) \\ {F}_{DA} ( t ) = \text{?} {X}_{j} \cdot {T}_{j} \text{?} & ( Eq . 2 ) \\ F ( t ) = {x}_{D} {F}_{D} ( t ) + {x}_{DA} {F}_{DA} ( t ) & ( Eq . 3 ) \\ {T}_{j} ( t ) = \text{?} {ρ}_{j} ( R ) - \text{?} A \text{?} exp ( \frac{- t}{\text{?}} \cdot [ 1 + ( \frac{\text{?}}{\text{?}} ) \text{?} ] ) dR & ( Eq . 4 ) \\ {ρ}_{j} ( R ) = \frac{1}{{σ}_{j} \cdot \sqrt{2 π}} exp ( \frac{- [ {R\_R}_{j} ] \text{?}}{2 {σ}_{j}^{2}} ) & ( Eq . 5 ) \\ \text{?} = {FWHM}_{j} ( 2 \sqrt{2 ln 2} ) . \\ \text{?} \text{indicates text missing or illegible when filed} & ( Eq . 6 )\end{matrix}$$

where F_{D} is the time-resolved fluorescence decay function of the GFP donor (Eq. 1), best-fit by a two exponential decay (FIGS. 12A-B). F_{DA} (Eq. 2) is the time-resolved fluorescence decay function of the GFP donor-acceptor FRET sample. F_{D} and F_{DA} were fit to a linear combination of mole fractions of x_{D} and x_{DA}. x_{D} equals zero for the intramolecular FRET sensor (Eq. 3). F_{DA} is a linear combination with molar fraction Xⱼ of two FRET-affected fluorescence decays Tⱼ(t) (Eq. 4). ρⱼ is the probability of each distance distribution, determined by least-squares minimization of the distance (nm) R, associated with each donor-acceptor lifetime species τᵢ (Eq. 5). (σⱼ) Gaussian interprobe distance distributions centered at Rⱼ=5.5 nm and 10.2 nm, with distribution widths defined by the standard deviation and full-width half-maximum (Eq. 6). The Förster distance (R₀) for the eGFP and tagRFP FRET pair is 5.8 nm. The parameters in this system of equations were optimized utilizing simultaneous least-squares minimization to waveforms from donor-only and donor-acceptor cell lines. The best-fit model was indicated by minimized χ² and by evaluation of the parameter error surface as described in our previous publications [18-20].

For spectral detection, the observed fluorescence emission spectrum was fitted by least-squares minimization to a linear combination of component spectra:

F_{Fit}(λ)=aF_{D}(λ)+bF_{A}(λ)+cF_{C}(λ)+dF_{W}(λ) (Eq. 13)

where D is donor, A is acceptor, C is cell autofluorescence, and W is water Raman, and a, b, c, d are the coefficients determined from the fit.

$$FRET = \frac{FR \times QR - AR}{1 + FR \times QR} \qquad ( Eq . 14 )$$

For an intramolecular FRET sensor, having both donor D and acceptor A, FRET was determined from (Eq. 14), where QR is the ratio of quantum yields (Q_{D}/Q_{A}) in the absence of FRET, AR is the ratio of molar absorptivities (ε_{A}/ε_{D}), both obtained from reported values [21]. QR is corrected for spectrograph sensitivity at the appropriate wavelength (FIG. 16). The only experimental observable in (Eq. 15) was FR.

$$FR = \frac{Acceptor}{Donor emission} = \frac{b {F}_{A}}{a {F}_{D}} \qquad ( Eq . 15 )$$

Full derivation of (Eq. 13)-(Eq. 15) can be found in the supplementary material.

### HTS Data Analysis

Fluorescent compounds were identified and flagged as potential false-positives by evaluating the similarity index (SI) between an observed compound spectrum I₁^{(a)} and DMSO control spectra I₁^{(b)}:

$$\begin{matrix} SI = 1 - \frac{\text{?} \cdot \text{?}}{\sqrt{\text{?} \cdot \text{?}} \sqrt{\text{?} \cdot \text{?}}} \\ \text{?} \text{indicates text missing or illegible when filed} & ( Eq .16 )\end{matrix}$$

The spectra of 192 DMSO control wells (% v/v DMSO) were averaged for each screen to generate a single control spectrum (I^{(b)}). The fluorescence spectra of the two-color SERCA biosensor, screened against 727 separate compounds (I^{(a} [symbol] ⁾), was compared with the single DMSO control spectrum. The similarity index between the spectra was computed over the GFP emission wavelength (i=500-540 nm). The fluorescent compound threshold was set to flag potential false-positives with an SI greater than 2×10⁻⁴ throughout the screens.

### Enzymatic SERCA Activity Assays of FRET Hits

Functional assays were performed using rabbit light skeletal sarcoplasmic (SR) vesicles [2]. An enzyme-coupled, NADH-linked ATPase assay was used to measure SERCA ATPase activity in 96-well microplates. Each well contained 50 mM MOPS (pH 7.0), 100 mM KCl, 5 mM MgCl₂, 1 mM EGTA, 0.2 mM NADH, 1 mM phosphoenol pyruvate, 10 IU/mL of pyruvate kinase, 10 IU/mL of lactate dehydrogenase, 1 μM of the calcium ionophore A23187 from Sigma (St. Louis, Mo.), and CaCl₂ added to set free [Ca²⁺] to 10 μM [22]. 4 μg/mL of SR vesicle, calcium, compound, and assay mix were incubated for 20 min. The assay was started upon the addition of ATP, at a final concentration of 5 mM (total volume to 200 μL), and absorbance read in a SpectraMax Plus microplate spectrophotometer from Molecular Devices (Sunnyvale, Calif.).

### Results

High Precision FRET Efficiency Determinations from Two Independent Fluorescence Measurements

The sarco/endoplasmic reticulum calcium ATPase (SERCA) cycles through multiple conformations as it pumps calcium into the sarcoplasmic reticulum. Briefly, a green fluorescent protein (GFP) was fused to the N-terminus of SERCA and a red fluorescent protein (RFP) was fused to a flexible intrasequence loop located on the nucleotide-binding domain of SERCA. The distance between these two fluorescent proteins can be measured by determining the rate of fluorescence resonance energy transfer (FRET). This two-color SERCA (2CS) biosensor was stably expressed in a human embryonic kidney (HEK293) cell line, and grown in sufficient quantities for high-throughput screening [2]. The binding of potential small-molecule effectors is directly evaluated as changes in FRET (FIG. 6B).

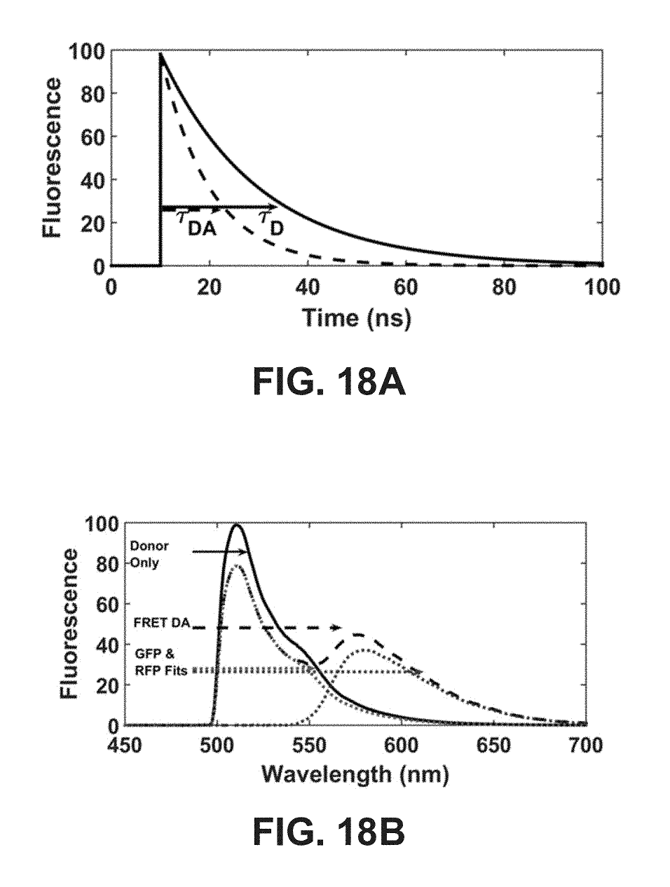

Conceptual fluorescence lifetime waveforms are depicted in FIG. 18A. In the actual lifetime measurements, typically 1000 laser pulses are averaged over a 200 ms interval, to generate an entire decay waveform for each well of a 384-well plate. The fluorescence lifetimes τ_{DA} (FRET biosensor with donor and acceptor) and τ_{D} (donor-only control) are used to determine the energy transfer efficiency FRET=1−τ_{DA}/τ_{D}. A representative complete fluorescence emission spectrum acquired with a 100 ms integration time at 0.5 nm spectral resolution in spectral mode is shown in FIG. 18B. The high-quality emission spectrum, acquired from a single well was decomposed into a linear combination of its spectral components green fluorescent protein (GFP) and red fluorescent protein (RFP). These components were then used to solve for the contribution of the fluorescence emission from GFP (donor fit) and RFP (acceptor fit), allowing for a high-precision determination of an ensemble-averaged FRET from the 2CS biosensor.

### Global Lifetime Analysis Resolves Structural Status of 2CS Biosensor

Global analysis of the fluorescence intensity decay rate (lifetime mode) was used to resolve two distinct structural states of the 2CS biosensor (Eq. 1)-(Eq. 6). These distinct structural states of 2CS were previously resolved using single-molecule fluorescence microscopy [12], which is not a high-throughput detection method. Here, we demonstrate analogous structural resolution of the 2CS biosensor, except with an ensemble-averaged FRET measurement, acquired in 200 ms per well from live-cell suspensions.

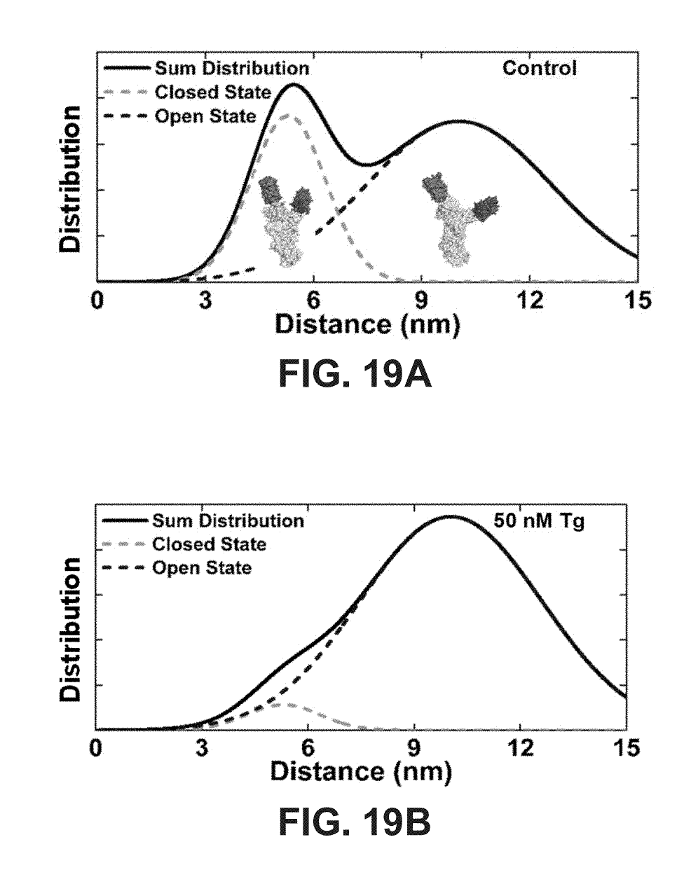

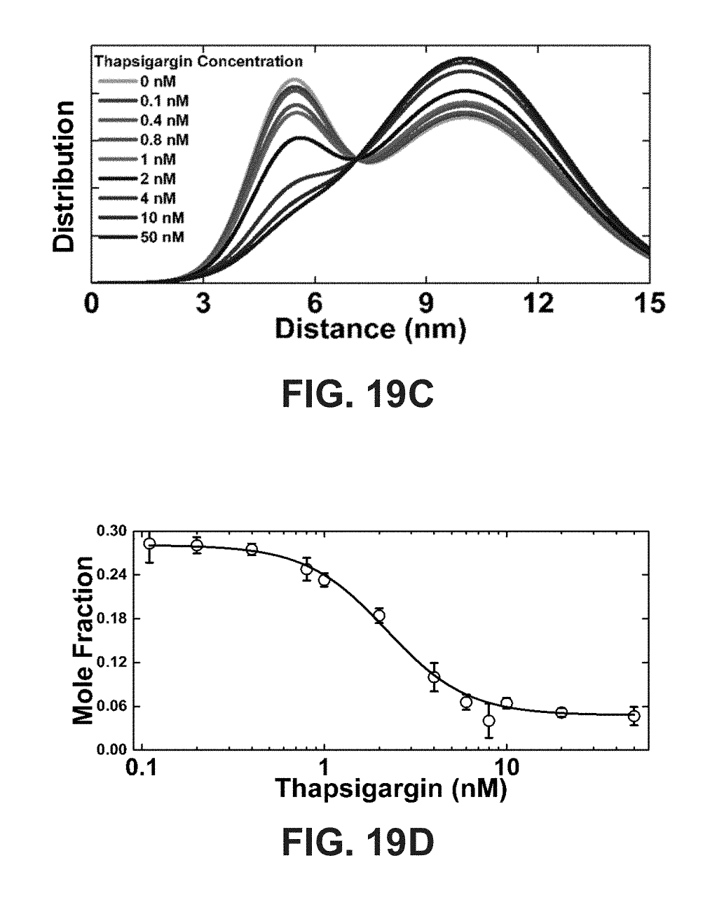

FRET is a sensitive spectroscopic molecular ruler, due to the R⁻⁶ distance dependence of the rate of energy transfer from an excited donor fluorophore (GFP) to an acceptor (RFP) in the ground state [24]. The exceptionally good precision of direct waveform recording (DWR) and global analysis of the fluorescence decay waveforms produced in this fashion allow FRET measurements to be evaluated in terms of distance distributions and mole fractions of physiologically-relevant structural states. Fluorescence lifetime waveforms were analyzed using a global two-component model (Eq. 1)-(Eq. 6), yielding a two-state structural model for SERCA's cytosolic headpiece, describing an equilibrium between the open (102 nm) and closed (55 nm) structural states with full-width at half-maximum (FWHM) of 69 and 22 nm; respectively. The Gaussian distance distribution was determined for each structural state and plotted as a histogram in FIG. 19A The distance R and FWHM fitting parameters were allowed to vary globally, and the mole fraction of each state was determined according to the two-component global fit (Eqs. 1-6). Thapsigargin inhibits SERCA at nanomolar concentration, and perturbs SERCA2a's cytosolic headpiece, greatly increasing the population of the more open and disordered structural state at 50 nM (FIG. 19B). The full concentration dependence is illustrated in (FIG. 19C), and the concentration-response curve of the closed state mole fraction (FIG. 19D) yields an EC₅₀ value of 2.2 nM thapsigargin in agreement with the known EC₅₀ for SERCA inhibition [25].

### Pilot Screening of NCC Libraries to Evaluate Both Spectral and Lifetime FRET Detection

A small-molecule library (National Clinical Collections 1&2), consisting of 727 compounds previously evaluated in preclinical and clinical trials, was used to evaluate both spectral and lifetime modes of the spectral unmixing plate reader (SUPR). After an initial quality control check of the 2CS cell line on each day of screening (response to known effectors and signal level), a HEK stable clone overexpressing the 2CS biosensor was dispensed, using a Multidrop liquid dispenser into 384-well plates, and then scanned in both spectral and lifetime modes after 20, 60, 90, and 120 minutes of incubation with the compounds or control wells.

A single-exponential fit was used to determine the lifetime MA from 2CS and m from the one-color SERCA2a donor-only control cell line. These lifetimes were used to determine FRET=1−τ_{DA}/τ_{D}. In spectral mode, the observed spectrum acquired from each well was decomposed into a linear combination of components (GFP, RFP, cellular autofluorescence, and water Raman). The reference spectrum of each was used to solve for the total contribution of fluorescence emission from each component (Eq. 13). These values were used to calculate a fitted ratio of the total fluorescence emission of RFP/GFP (Eq. 15) and then FRET from the 2CS biosensor was determined using the simplified FRET equation for intramolecular FRET sensors as described in the preceding article (Eq. 14) [17].

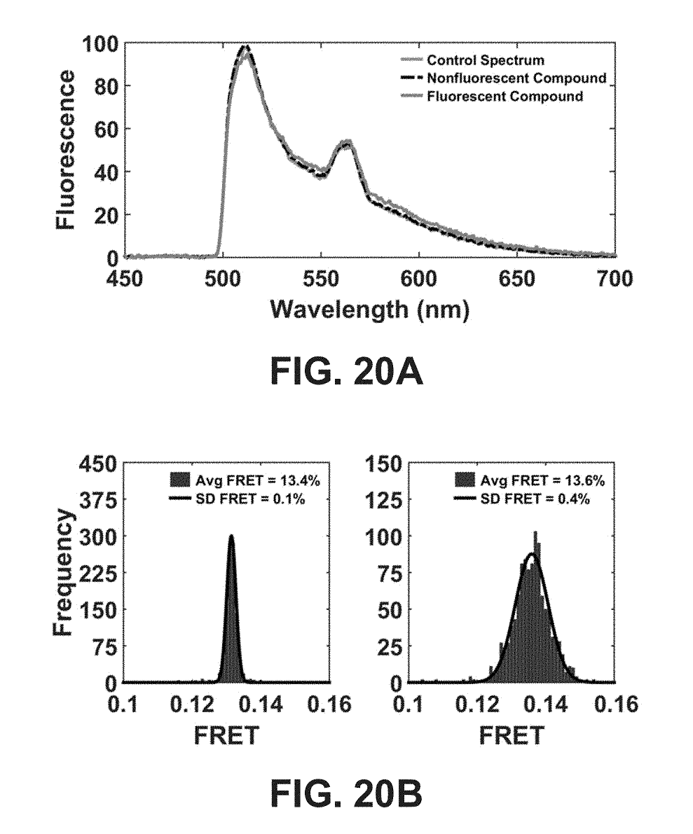

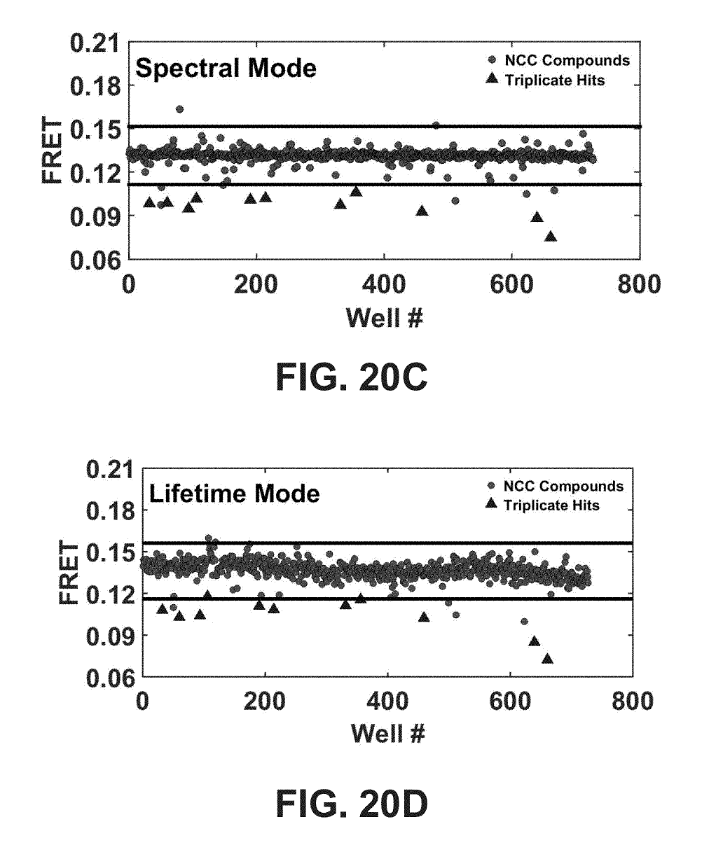

Both lifetime and spectral fluorescence measurements are prone to interference from fluorescent compounds. We took advantage of the information contained in the full emission spectrum to develop a streamlined process to flag these potential false-positives. A spectral similarity index (Eq. 16), which monitors differences from the spectra of control wells with no compound added, was computed in the donor only region. A stringent similarity index threshold (2×10⁻⁴) was used to flag 44 compounds as potential false-positives, due to interference from compound fluorescence (FIG. 20A).

Histogram plots from all wells that passed the fluorescence compound filter, from a single NCC screen demonstrate a three-fold increase in precision from the spectral mode, in comparison to lifetime mode, as exemplified by a narrower distribution (FIG. 20B). These two complementary determinations of FRET were used to identify hits, quickly rule out false-positives, and increase reproducibility across screens.

From three independent screens of the NCC libraries at a time point of 20 min after compound incubation, eleven reproducible FRET hits were found. These hits were identified using the spectral unmixing method and a hit threshold set at a 0.02 change in FRET. The results from one 2CS NCC screen are depicted in FIGS. 20C and 20D. Based on triplicate screens, 11 out of 16 compounds were identified as reproducible FRET hits (shown as blue circles in FIG. 20C). In lifetime mode, 9 of the 11 reproducible FRET hits found using spectral mode were also reproducible lifetime hits (FIG. 20D).

### Reproducibility of Hit Identification Across Independent Screens and Time Course Studies

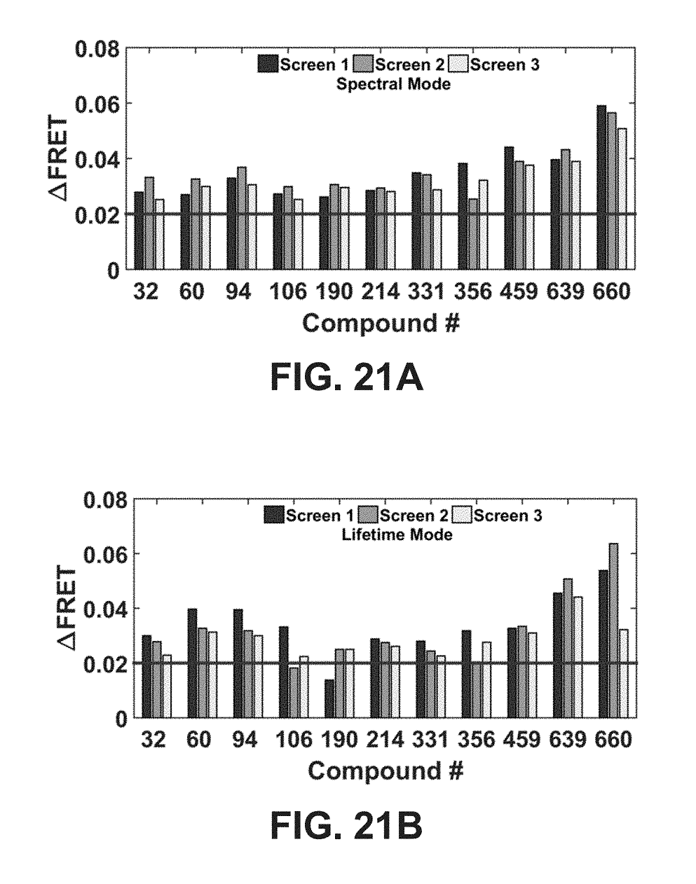

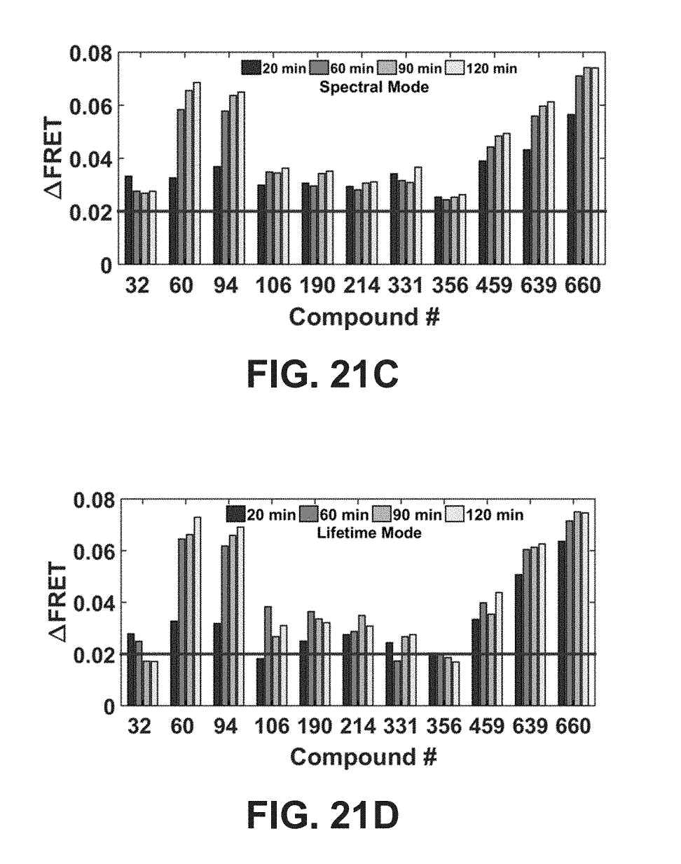

The reproducibility of 2CS FRET hits identified across three independent screens in spectral mode, after 20 min compound incubation at 10 μM concentration, is depicted in FIG. 21A. The change in 2CS FRET (Δ FRET) of each hit from three independent NCC screens remained nearly constant from one screen to the next. For purposes of directly evaluating the use of the spectral recording method, a hit threshold of a 0.02 negative change in FRET (horizontal line) was applied and will be used from here on. When these same hits were evaluated in lifetime mode, nine of the eleven spectral FRET hits were found to be reproducible hits (FIG. 21B). Compounds #106 and 190 were not identified as lifetime FRET hits in one of the three independent screens.

The ability to acquire lifetime and spectral measurements with scan times under three minutes for an entire 384-well microplate, allowed for the examination of 2CS FRET changes in response to the full NCC library of compounds at multiple compound incubation time points. Time-course screening may not be directly amenable to large-scale screening but is potentially highly applicable for assessing the reproducibility of a large number of FRET hits identified during a large-scale HTS campaign, at multiple concentrations. Time-dependent compound effects may also elucidate compounds with low binding affinities or delayed effects from low membrane permeability. These types of studies may also be useful for other fluorescence bioassays solely based on monitoring time-dependent effects, as we will depict later using the cameleon calcium FRET sensor. For these 2CS pilot screening studies, time-dependent screening was used to determine the inter-screen reproducibility of FRET hit identification as assessed both spectral and lifetime modes.

Time-dependent scans of reproducible 2CS FRET hits were consistent over multiple time points (20, 60, 90, and 120 min after compound incubation). The 2CS FRET hits analyzed here were from screen 2 (turquoise bars in FIGS. 21A and 21B) and show excellent reproducibility across time points. Five compounds #60, 359, 459, and 639 exhibited an increased FRET change over time (FIG. 21C. Reproducible hits were evaluated in lifetime mode (FIG. 21D) Very subtle differences in the 2CS FRET changes were found across spectral and lifetime methods. Ten compounds from screen 2 were identified as hits, after 20 minutes of compound incubation. Compounds #32 and 356 displayed a modest reduction in 2CS ΔFRET at the later time points. Compounds #60, 94, 459, 639, and 660 again exhibited an increased FRET change over time. Overall, both methods show excellent agreement in terms of the direction and magnitude of FRET change.

### Multi Parameter Concentration-Dependent Effects of FRET Hits

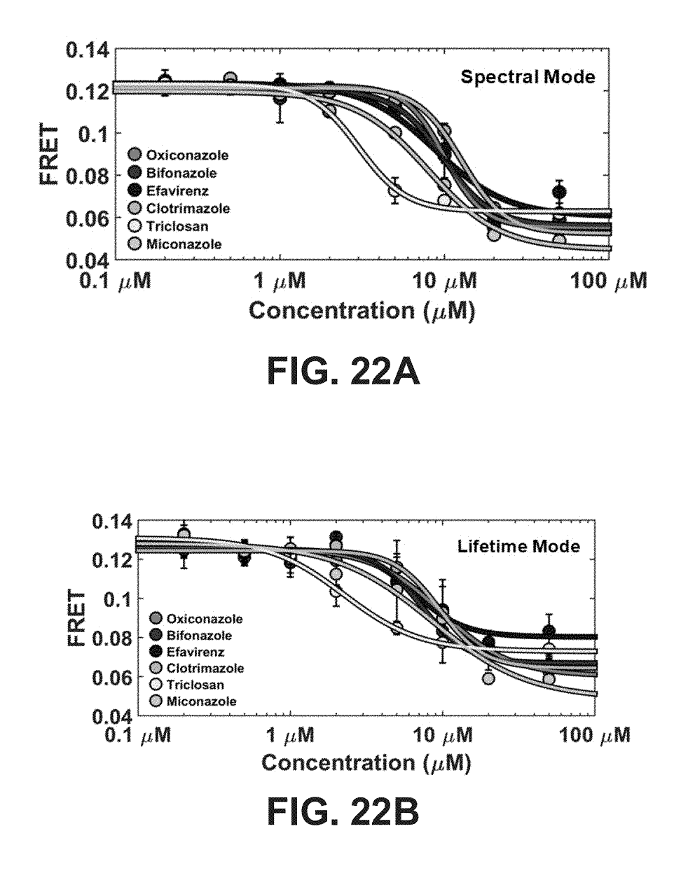

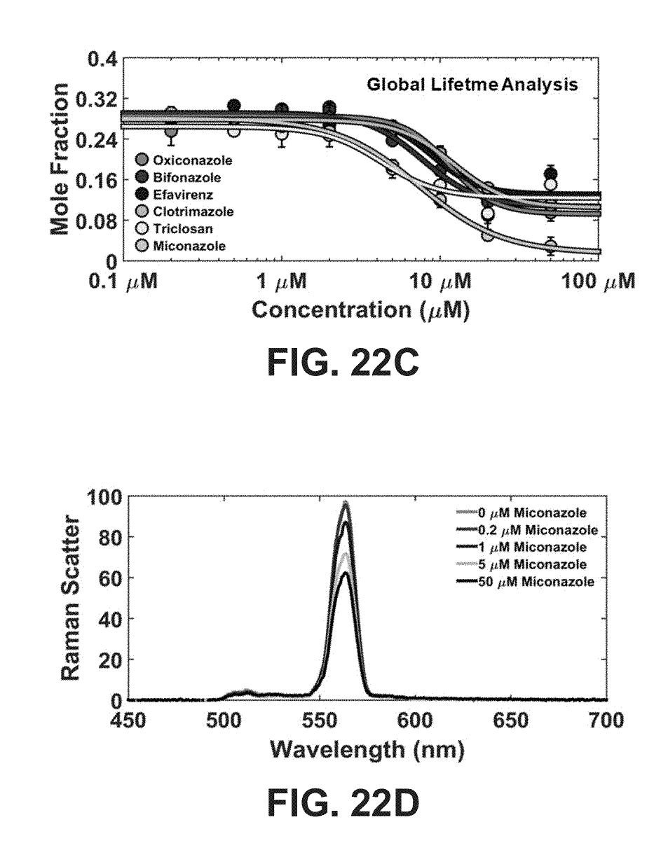

The reproducible 2CS FRET hits were further evaluated as a function of concentration. Compounds were dispensed into 384-well plates, across an eight-point concentration-gradient (n=4 for each concentration). Three independent dose-dependent FRET tests were performed on the 2CS FRET hits. Six compounds that produced the largest reproducible FRET change after 120 minute compound incubation are depicted. These compounds dose-dependently decreased 2CS FRET as determined by spectral mode (FIG. 22A). Each FRET curve was fit using the Hill equation. Decreased 2CS FRET was observed at micromolar concentrations with notable differences in the apparent EC₅₀ (half maximal effective concentration) of the FRET curve. The same 384 well plate, containing the reproducible hits, was evaluated in lifetime mode with a simple, single exponential fit (Eq. 1-2) and demonstrated great agreement between the FRET change across the two modes of independent FRET measurements. (FIG. 22B).

The compounds found to be reproducible hits and that exhibited dose-dependent FRET changes all decreased 2CS FRET. The imidazole antifungal clotrimazole was a hit and has been previously shown to inhibit SERCA function [26]. The related compounds oxiconazole, bifonazole, and miconazole were also hits. The antibacterial triclosan has been shown to increase cytosolic calcium [27] but to our knowledge not through interaction with SERCA.

Global analysis of the lifetime data (Eq. 1)-(Eq. 6) resolved a dose-dependent change in the mole fractions of the open and closed structural states. Using this distance distribution model, the reproducible hits perturbed the 2CS structural equilibrium between open and closed states in FIG. 22C (5.5 nm distance distribution shown). The high sensitivity of spectral mode is shown by the fluorescence signal of the water Raman spectrum. Raman scattering was acquired from compound-only wells of the known aggregator miconazole [28]. Compound aggregation dose-dependently causes more light to be absorbed and decreases inelastic light scattering (Raman band) (FIG. 22D). This information may become useful for flagging potential false-positives due to compound aggregation.

### Functional Characterization of FRET Hits on SERCA ATPase Activity and ER Calcium Content

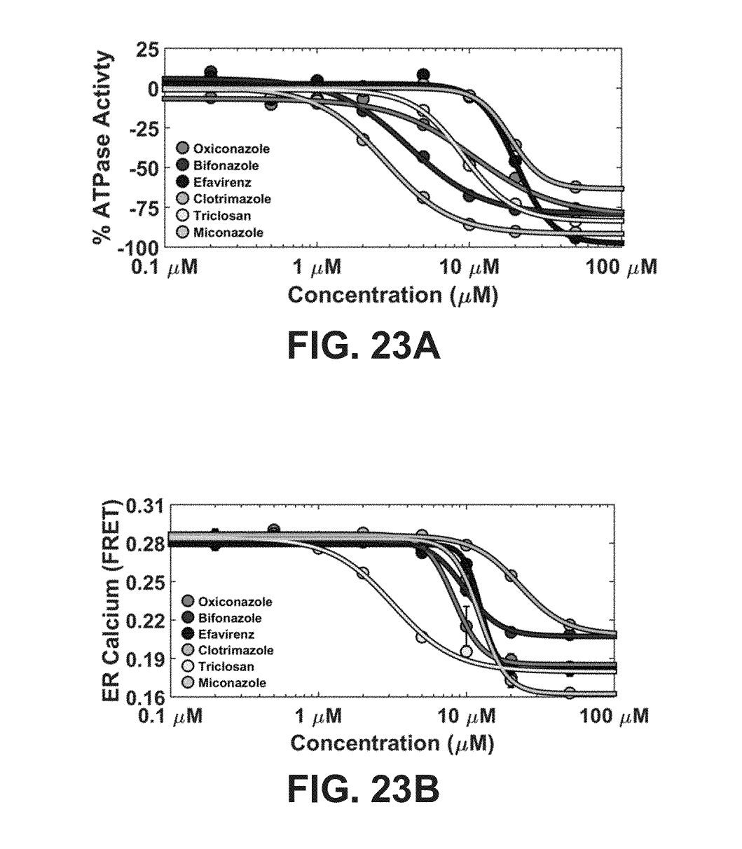

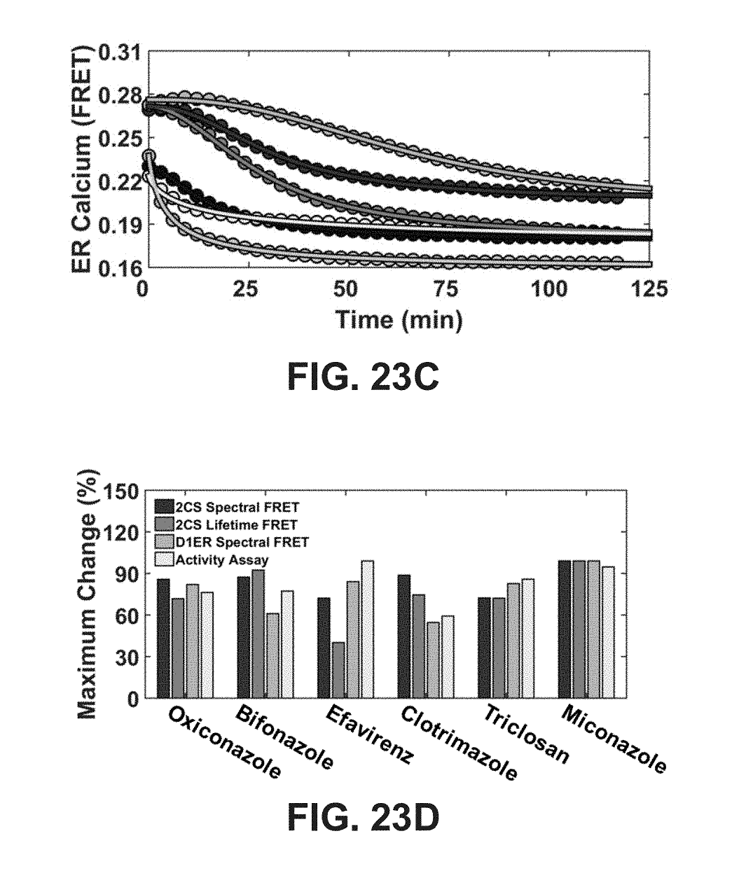

Functional assays of the confirmed reproducible NCC hits were used to assess the relationship of hits that perturb 2CS structure and their effects on SERCA function. The ATPase activity of purified SERCA was measured after 20 minute incubation with a saturating dose of buffered free calcium (10 μM) and titration of each compound. Experiments were performed in triplicate with eight-point concentration curves. The top six hits were found to dose-dependently inhibit SERCA's ATPase function (FIG. 23A). The antifungal Miconazole shows almost complete inhibition (92.4%) with a Kᵢ of 2.8 μM. Clotrimazole's ability to inhibit SERCA's ATPase was slightly reduced in comparison with a Kᵢ of 17.3 μM which is in agreement with previous steady-state measurements (7-35 μM) [26].

SERCA malfunction can result in decreased ATPase activity and/or calcium pumping efficiency. ER calcium content was monitored over time using the endoplasmic-localized cameleon calcium FRET sensor (D1ER) [15]. As demonstrated in the preceding article, known SERCA inhibitors deplete ER calcium in a time and dose-dependent manner and can be monitored using live-cells expressing D1ER. Briefly, D1ER FRET changes were monitored over time by repeatedly (every 3 minutes) scanning a 384-well plate containing varying concentrations of the 2CS FRET hits. D1ER cells were assessed immediately after compound incubation. These plates were scanned only in spectral mode using 434 nm excitation with a laser-driven light-source, to acquire a full emission spectrum from each well. The appropriate CFP/YFP reference spectra were used to determine FRET using (Eqs. 2-4).

D1ER FRET curves were determined for each time-point scan, after compound incubation, over a period of 120 minutes (40 scans total). The 2CS FRET hits displayed time-dependent and compound-specific ER calcium depletion. Miconazole (turquoise) exhibited both maximal SERCA ATPase Vmax inhibition and the largest amount of ER calcium depletion as depicted at the final 120 min time point in FIG. 23B. Maximal ER calcium depletion in the presence of a saturating dose (50 μM) of each compound (decreased D1ER FRET) was assessed over a 120 minute period (FIG. 23C). The 2CS FRET hits displayed time-dependent and compound-specific ER calcium depletion. The K_{I} and EC₅₀'s from the ATPase activity and D1ER FRET curves showed good agreement at the 20 minute time points. The structure-activity relationship of the 2CS FRET hits was further analyzed by comparing 2CS FRET (both spectral and lifetime mode), ATPase activity, and ER calcium depletion (FIG. 23D). The maximal change (shown as percent change) of the structural FRET change from the 2CS FRET biosensor had excellent agreement with the maximal change from two different functional assays (ATPase activity assay and ER calcium depletion). Structural perturbation of the 2CS FRET biosensor directly relates to a compound's effect on SERCA function.

### Discussion

This study illustrates the complementary combination of spectral and lifetime fluorescence detection for the purposes of HTS. The spectral unmixing method increases the precision of hit identification and reproducibility of the hits in concentration-response curves (FIGS. 21A-D). The fluorescence lifetime detection mode offers excellent precision and offers the additional advantage of structural resolution, revealed by multi-exponential global lifetime fitting. This approach resolves multiple FRET populations and assesses them in terms of distance-distributions and mole fractions, assigned to structurally-relevant perturbations of SERCA effectors (FIGS. 19A-D). This resolution of multiple FRET-detected structural states from a live-cell biosensor is highly advantageous for screening, offering the potential to elucidate chemotypes or classes of compounds, identified in large-scale screens, which differentially alter the structural status of a biosensor. This high-content information can be used to generate structure-activity relationships based on binding-affinities, structural dynamics, and disorder.

The capability to couple two independent measurements of FRET, thereby substantially decreasing the false-positive rate, would be of significant value to the high-throughput screening community. Spectral recording does not offer the resolution of structural information, in terms of resolving multiple structural states, but can be used to identify fluorescent compounds, eliminate artifacts due to dispenser error or contaminated samples, and increase assay precision across screens (FIGS. 21A-D).

This is the first microplate reader capable of direct waveform recording in both lifetime and spectral domains. A recent review of fluorescence lifetime imaging (FLIM) plate readers demonstrates the medium-throughput capabilities currently offered by other technologies (20 min scan times per 96 well plate) [29]. The approach described here is considerably faster, yet offers very high precision.

Beyond developing new fluorescence technology, the overarching goal of this research is to identify novel small-molecule SERCA effectors with therapeutic potential for multiple disease states. These studies employed a 2CS biosensor based on the SERCA2a isoform [2], which is the primary isoform expressed in the heart. We have engineered constructs based on the other human isoforms, with the intent of performing drug-discovery campaigns to identify isoform-specific SERCA effectors. Further, we are currently developing new synthetic analogues based on our previously identified SERCA activators and inhibitors, where our HTS approach allows us to quickly assess and triage the most prominent candidates from a large pool of synthetically-derived analogues. We concluded this assay-based demonstration by investigating hits identified through structural-based screening, using an NADH-enzyme coupled ATPase activity assay and the D1ER endoplasmic reticulum-targeted calcium sensor to evaluate the correlation between the structure and function of hits identified in this pilot screening campaign (FIGS. 23A-D).

The compounds identified during these pilot screens all decreased FRET from the 2CS biosensor, corresponding to opening of SERCA's cytoplasmic headpiece. This may be a consequence of their similarity in the mode of binding or mechanism of inhibition. However, the 2CS biosensor is not limited to detection of decreases in FRET. In broken cells, we have previously shown that ligands such as calcium increase FRET, due to closure of SERCA's cytoplasmic headpiece [23]. It is plausible that the maximal FRET effect is essentially reached for the HEK293 live-cells, in which calcium and ATP maintain SERCA in its closed structural state.

The novel paradigm used in the present study enables the measurement of multiple FRET parameters. These high-content assays are ideally suited for high-throughput screening campaigns, with potential to discover novel allosteric effectors, which may differentially perturb FRET. This strategy is now being evaluated for use on homogenate and microsomal cellular preparations using FRET-based biosensors. These applications allow for fine control of environment (calcium, nucleotide, pH, etc.). Preliminary results have demonstrated that these purified preparations of FRET-based biosensors are suitable for counter screens and also in-depth structural evaluations of novel SERCA effectors.

The high-resolution FRET approach, coupled to functional assays, is applicable to a wide range of protein targets, including the ryanodine receptor [30], myosin [19, 31], phospholamban [10], multiple-drug resistance receptor [32], and the tumor necrosis receptor [33]. The ability to quickly and reliably assess structural perturbations from biosensors in relation to physiologically-relevant functional changes holds high promise for the development of allosteric effectors and potentially valuable lead compounds.

### Supplementary Material

### Reference Spectra Determination for Fluorescence Emission Components of 2CS FRET Biosensor

The spectral unmixing process yields a decomposition of the observed (experimental) spectrum into a linear combination of biochemically meaningful component spectra. In the case of the experiments reported here, a minimum of four components (basis spectra) must be included in the spectral fitting: donor fluorescence (GFP), acceptor fluorescence (RFP), cellular autofluorescence, and Raman scattering of the buffer (effectively the inelastic Raman scattering of water). Scattered exciting light, stray light, background fluorescence of the plate material, and impurities are other possibilities. The quality of the fit is typically judged by the residuals (errors) between the observed and fitted spectrum. A challenge that was overcome was that it is not possible to prepare pure samples of the individual components except in the case of the water Raman.

### Water Raman:

A signal-to-noise comparison of the inelastic light scattering due to the water Raman band after excitation at 473 nm (GFP excitation) was performed. Comparison water Raman spectra were acquired on three commercially-available fluorescence microplate readers equipped with emission monochromators. The acquisition rate in these experiments (FIG. 12A) was set at one second per wavelength, and the wavelength spacing at 1 nm. A water Raman spectrum (black) was also acquired on a cuvette-based fluorescence spectrometer (Cary Eclipse, xenon flash-lamp excitation) with the same acquisition time and wavelength interval (FIG. 12B). FIG. 12C shows the superposition of six spectra, each corresponding to a different well of a 384 plate, at 200 ms acquisition time per well, acquired with the spectral unmixing plate reader (Fluorescence Innovations). The spectra have been normalized to the same area under the curve to better illustrate the very high repeatability. The relatively narrow water Raman spectra acquired with the spectral unmixing plate reader, allows for the capability to extract spectral features, and produce robust and high precision measurements. Note, too, the very low background signal from the black polypropylene plates on either side of the water Raman band, despite the epi-illumination geometry.

### Cellular Autofluorescence

Excitation of living cells in the 450-500 nm wavelength range produces a background autofluorescence signal, generally attributed to flavins [34]. The fluorescence spectrum of 50 μL of untransfected HEK293 cells suspended in PBS at a concentration of 50,000 cells per well of a 384 well plate is shown in FIG. 13. The sharp and narrow peak of the water Raman feature at 560 nm was used to subtract the water Raman signal from cellular autofluorescence spectrum, thereby producing the autofluorescence reference spectrum.

GFP reference spectrum. The reference spectra of GFP (eGFP) were acquired from purified fluorescent protein preparations, and by subtracting each component spectrum (water Raman, autofluorescence, and the signal from fluorescent protein) using live-cells expressing the fluorescent proteins. The former approach avoids the need to consider the autofluorescence contribution, but the signal of the green or red fluorescent proteins may be dependent on buffer conditions, and vary with environment surrounding the purified protein.

The reference spectrum of GFP, the donor fluorescent protein used for the red/green FRET pair of the two-color SERCA biosensor, was acquired from HEK293 cells overexpressing high levels of an appropriately donor-labeled control cell line. This cell line expressed a genetically-encoded construct, where GFP was fused to an intra-sequence flexible loop on SERCA2a's cytosolic nucleotide-binding domain (at residue 509). The GFP reference spectrum was generated by subtracting the contribution of the cellular autofluorescence and water Raman from the observed GFP SERCA control cell line (dark green spectrum in FIG. 14). The fluorescence signal of the GFP-SERCA fusion protein was much higher than the background signal from the cells themselves (cellular autofluorescence). Therefore, a very subtle subtraction of the autofluorescence signal was made for this particular cell line. However, the shift in the shape of the subtracted GFP reference fluorescence spectrum did not significantly alter the results of the spectral unmixing methods. This reference spectrum was used to accurately resolve the fluorescence signal of GFP from a cell line over-expressing the two-color SERCA FRET biosensor. Further comparison of the inferred GFP reference spectrum from live-cells with the spectrum of purified protein is shown in FIG. 14, by the overlay of GFP subtraction and the denoted solved GFP reference spectrum. Although small variations in the overall shape of the reference GFP spectrum acquired from live-cells and purified protein preparations have been previously reported [34], these differences did not distort the results obtained from the spectral unmixing methods.

RFP reference spectrum. The reference spectrum of the red fluorescent protein (tagRFP) was acquired using 532 nm laser excitation, which eliminated the contribution from cellular autofluorescence. Excitation at a longer wavelength also shifts the water Raman band to the 650 nm wavelength. The effect of the water Raman band is much weaker because of its χ⁴ dependence. Finally, the RFP-SERCA fluorescent fusion construct had much higher expression than the intrasequence labeled GFP-SERCA control construct. The RFP reference spectrum is shown in FIG. 15.

The four reference spectra shown in SUPR FIG. 6A were used for all studies involving spectral unmixing of the 2CS construct with GFP and RFP-labeled fluorescent proteins.

### Derivation of the Spectral FRET Equation for 2-Color Biosensor

### Nomenclature:

ε_{A}: molar absorptivity of acceptor at λₑₓ

ε_{D}: molar absorptivity of donor at λₑₓ

Φ_{FRET}: Fluorescence quantum yield of FRET efficiency

Φ_{F,D}: Fluorescence quantum yield of donor, including potential change due to FRET

Φ_{F,A}: Fluorescence quantum yield of acceptor

### Assumptions:

1. # Donor excited states proportional to ε_{D}

2. # Acceptor excited states proportional to ε_{D} Φ_{FRET}+ε_{A}

3. # Donor emission events proportional to ε_{D} Φ_{F,D}

4. # Acceptor emission events proportional to (ε_{D} Φ_{FRET}+ε_{A})Φ_{F,A}

### Derivation:

Let FR be the FRET ratio of total number of photons emitted by acceptor to total number of photons emitted by donor (Eq. 5); assuming the CCD linear-array spectrograph detector has been corrected for wavelength dependence of its response. The total fluorescence emission F of each component's reference spectrum is dependent on the scalar coefficients shown as the sum of the linear combination of the four components of the fluorescence emission signal from as shown in (Eq. 13), where b represents the scalar coefficient for RFP (acceptor), and a the scalar coefficient for GFP (donor).

$$FR = \frac{Acceptor fluorescence}{Donor fluorescence} = \frac{b {F}_{RFP}}{a {F}_{GFP}} \qquad EQ . 12$$

Substitution of extinction coefficients (molar absorptivity) and quantum yields, which represent the total acceptor and donor fluorescence from the FRET biosensor gives following equation:

$$FR = \frac{( ɛ D Φ FRET + ɛ A ) Φ F , A}{ɛ D Φ F , D} . \qquad Supp Eq . 3$$

However, the contribution of FRET to the donor fluorescence when the rate of fluorescence energy transfer is zero, gives Φ_{F,D}=Φ_{F,D}(E=0) (1−Φ_{FRET}). Substitution of this equation for the donor quantum yield at (E=0) yields:

$$FRET = \frac{FR * Φ F , D / Φ F , A -}{1 + FR * Φ F , D / Φ F , A} . \qquad Supp Eq . 4$$

where QR (ΦF,D/ΦF,A) is the ratio of the donor fluorescence quantum yield under non-FRET conditions to the acceptor fluorescence quantum yield. AR (εA/εD) is the extinction coefficient ratio of acceptor molar absorptivity to the donor molar absorptivity at 473 nm wavelength excitation. Note that FR is the only experimentally derived quantity as the quantum yields and extinction coefficients of the eGFP and tagRFP have been previously reported, and were determined for 473 nm wavelength excitation [35].

The quantum yield ratio could be determined directly, although quantum yields are notoriously difficult to measure with very high accuracy. Moreover, the fluorescence ratio (FR) depends on the wavelength dependence of the CCD spectrograph detector. In other words, the measured FR needs to be corrected to a constant detector response (no wavelength dependence).

A β-factor was experimentally determined, and used to calibrate for the difference in the overall spectrograph sensitivity at longer wavelengths (wavelength-dependent response and the wavelength dependence of the grating's diffraction efficiency) The dispersion of the light by the grating is non-linear, so shorter wavelength light is spread out more than longer wavelength light. For simplicity the β-factor was designated to quantum yield factor (QR) yielding:

$$FRET = \frac{β ( QR ) * FR - ( \text{?} A / \text{?} D )}{1 + β ( QR ) * FR} . \\ \text{?} \text{indicates text missing or illegible when filed} \qquad Supp Eq . 5$$

The β value (correction factor) used to correct for difference in GFP and RFP detector sensitivity was determined using the fluorescence lifetime data of the m (GFP-SERCA) and MA (2CS) cell lines, and solving for the apparent FRET efficiency in Supp Eq. 4).
Solving for the FRET efficiency of 2CS (above) for β gives

β=[(τD/τDA)(1+εA/εD)−1]/FR Eq. 6

where the τ values are fluorescence lifetimes acquired from the appropriate donor-only and donor-acceptor (FRET) cell lines.

FIG. 16 shows a single β-value (corrected QR=0.83) that gives excellent agreement between the FRET efficiency values calculated by lifetime and by spectra. The data used for this calibration of FRET efficiency was from the 12-point thapsigargin 2CS dose-response studies. Each point on the plot shows the FRET efficiency obtained from lifetime mode (direct determination using (Eq. 14), and after solving for the β-factor using the spectral FRET efficiency equation (Eqs. 5 and 6) at 12 different concentration of Thapsigargin. For all FRET pairs, each correction factor was obtained by using reported values of the fluorescent protein quantum yield and molar absorptivity [35], and correcting for CCD detector sensitivity. The β-factors for the CFP/YFP variants were not determined by direct comparison to fluorescence lifetime measurement, because a microchip pulsed laser with the appropriate excitation wavelength does not exist. The β-factor for the quantum yield ratio of the mCerulean and mVenus FRET pair was determined by using the known FRET efficiency of the C32V construct [36], and solving for the β-factor to match the expected value. This β-factor was then used to determine the FRET efficiency of the C17V and C5V construct as shown in FIG. 11B. The beta-factor for the eCFP/mcitrine FRET pair was determined by estimating the FRET efficiency of the D1ER FRET biosensor, under normal cellular conditions, using previously reported fluorescence lifetime measurements [37]. QR in supplemental table is the β-factor corrected quantum yield ratio from each FRET pair.

TABLE 2
Quantum Yield and Molar Absorptivity Ratios
for Donor and Acceptor FRET Pairs

| FRET pair | QR | AR |
|---|---|---|
| eGFP and tagRFP (2CS) | 0.83 | 0.05 |
| eCFP and mCitrine (D1ER) | 0.46 | 0.12 |
| mCerulean and mVenus | 0.37 | 0.10 |

### Multiple-Exponential Lifetime Fitting

The donor-only control cell line, where SERCA2a is fused to GFP, was used to determine the FRET efficiency using E=1−τ_{DA}/τ_{D}. The fluorescence waveform was fit using both one- and two-exponential model as shown in FIGS. 17A-B. The χ² of each fit across the nanosecond domain was determined from the sum of the residuals obtained from the fit. The χ² was reduced for the two-exponential model and this model was used for subsequent global lifetime analysis to determine distance distributions. Adding more than two exponentials to the model was not found to increase the goodness of the fits.
1 Cornea, R. L., et al., High-throughput FRET assay yields allosteric SERCA activators. J Biomol Screen, 2013. 18(1): p. 97-107.
2. Gruber, S. J., et al., Discovery of enzyme modulators via high-throughput time-resolved FRET in living cells. J Biomol Screen, 2014. 19(2): p. 215-22.
3. Bleeker, N. P., et al., A Novel SERCA Inhibitor Demonstrates Synergy with Classic SERCA Inhibitors and Targets Multidrug-Resistant AML. Mol Pharm, 2013.
4. Kang, S., et al., Small Molecular Allosteric Activator of the Sarco/Endoplasmic Reticulum Ca2+-ATPase (SERCA) Attenuates Diabetes and Metabolic Disorders. The Journal of biological chemistry, 2015.
5. Brini, M. and E. Carafoli, Calcium pumps in health and disease. Physiological reviews, 2009. 89(4): p. 1341-78.
6. Zsebo, K., et al., Long-term effects of AAV1/SERCA2a gene transfer in patients with severe heart failure: analysis of recurrent cardiovascular events and mortality. Circulation research, 2014. 114(1): p. 101-8.
7. Greenberg, B., et al., Prevalence of AAV1 neutralizing antibodies and consequences for a clinical trial of gene transfer for advanced heart failure. Gene therapy, 2015.
8. Lockamy, E. L., et al., Functional and physical competition between phospholamban and its mutants provides insight into the molecular mechanism of gene therapy for heart failure. Biochemical and biophysical research communications, 2011.
9. Ha, K. N., et al., Controlling the Inhibition of the Sarcoplasmic Ca2+-ATPase by Tuning Phospholamban Structural Dynamics. The Journal of biological chemistry, 2007. 282(51): p. 37205-14.
10. Gruber, S. J., S. Haydon, and D. D. Thomas, Phospholamban mutants compete with wild type for SERCA binding in living cells. Biochemical and biophysical research communications, 2012. 420(2): p. 236-40.
11. Hou, Z., et al., 2-Color calcium pump reveals closure of the cytoplasmic headpiece with calcium binding. PLoS One, 2012. 7(7): p. e40369.
12. Pallikkuth, S., et al., Phosphorylated Phospholamban Stabilizes a Compact Conformation of the Cardiac Calcium ATPase. Biophys J, 2013. 105(8): p. 1812-21.
13. Smolin, N. and S. L. Robia, A structural mechanism for calcium transporter headpiece closure. J Phys Chem B, 2015. 119(4): p. 1407-15.
14. Petersen, K. J., et al., Fluorescence lifetime plate reader: resolution and precision meet high-throughput. Rev Sci Instrum, 2014. 85(11): p. 113101.
15. Palmer, A. E., et al., Bcl-2-mediated alterations in endoplasmic reticulum Ca2+ analyzed with an improved genetically encoded fluorescent sensor. Proceedings of the National Academy of Sciences of the United States of America, 2004. 101(50): p. 17404-9.
16. Palmer, A. E. and R. Y. Tsien, Measuring calcium signaling using genetically targetable fluorescent indicators. Nature protocols, 2006. 1(3): p. 1057-65.
17. Schaaf, T. M., et al., Spectral Unmixing Plate Reader: High-Throughput, High Precision FRET Assays in Living Cells. Journal of biomolecular screening, 2016.
18. Muretta, J. M., et al., The structural kinetics of switch-1 and the neck linker explain the functions of kinesin-1 and Eg5. Proceedings of the National Academy of Sciences of the United States of America, 2015. 112(48): p. E6606-13.
19. Muretta, J. M., et al., Direct real-time detection of the structural and biochemical events in the myosin power stroke. Proceedings of the National Academy of Sciences of the United States of America, 2015. 112(46): p. 14272-7.
20. Li, J., et al., Structural and functional dynamics of an integral membrane protein complex modulated by lipid headgroup charge. J Mol Biol, 2012. 418(5): p. 379-89.
21. Thorn, T. L. a. K. Fluorescent protein properties. 2015; Available from: http://nic.ucsf.edu/FPvisualization/.
22. Mueller, B., et al., Direct detection of phospholamban and sarcoplasmic reticulum Ca-ATPase interaction in membranes using fluorescence resonance energy transfer. Biochemistry, 2004. 43(27): p. 8754-65.
23. Hou, Z., et al., Two-color calcium pump reveals closure of the cytoplasmic headpiece with calcium binding. PLoS One, 2012: p. accepted.
24. Lakowicz, J., Principles of Fluorescence Spectroscopy, 3rd ed. Springer 2006.
25. Rogers, T. B., et al., Use of thapsigargin to study Ca2+ homeostasis in cardiac cells. Bioscience reports, 1995. 15(5): p. 341-9.
26. Bartolommei, G., et al., Clotrimazole inhibits the Ca2+-ATPase (SERCA) by interfering with Ca2+ binding and favoring the E2 conformation. The Journal of biological chemistry, 2006. 281(14): p. 9547-51.
27. Cherednichenko, G., et al., Triclosan impairs excitation-contraction coupling and Ca2+ dynamics in striated muscle. Proceedings of the National Academy of Sciences of the United States of America, 2012. 109(35): p. 14158-63.
28. Seidler, J., et al., Identification and prediction of promiscuous aggregating inhibitors among known drugs. Journal of medicinal chemistry, 2003. 46(21): p. 4477-86.
29. Guzman, C., C. Oetken-Lindholm, and D. Abankwa, Automated High-Throughput Fluorescence Lifetime Imaging Microscopy to Detect Protein-Protein Interactions. Journal of laboratory automation, 2016. 21(2): p. 238-45.
30. Svensson, B., et al., FRET-based trilateration of probes bound within functional ryanodine receptors. Biophys J, 2014. 107(9): p. 2037-48.
31. Colson, B. A., et al., Site-directed spectroscopy of cardiac myosin-binding protein C reveals effects of phosphorylation on protein structural dynamics. Proceedings of the National Academy of Sciences of the United States of America, 2016. 113(12): p. 3233-8.
32. Iram, S. H., et al., ATP Binding Cassette Transporter Structure Changes Detected by Intramolecular Fluorescence Energy Transfer for High-Throughput Screening. Mol Pharmacol, 2015. 88(1): p. 84-94.
33. Lewis, A. K., et al., Open and closed conformations of the isolated transmembrane domain of death receptor 5 support a new model of activation. Biophys J, 2014. 106(6): p. L21-4.
34. Ogikubo, S., et al., Intracellular pH sensing using autofluorescence lifetime microscopy. J Phys Chem B, 2011. 115(34): p. 10385-90.
35. Cranfill, P. J., et al., Quantitative assessment of fluorescent proteins. Nature methods, 2016. 13(7): p. 557-62.
36. Koushik, S. V., et al., Cerulean, Venus, and VenusY67C FRET reference standards. Biophysical Journal, 2006. 91(12): p. L99-L101.
37. Avezov, E., et al., Lifetime imaging of a fluorescent protein sensor reveals surprising stability of ER thiol redox. J Cell Biol, 2013. 201(2): p. 337-49.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for identifying a compound that alters fluorescence resonance energy transfer (FRET) of a protein comprising:
a. providing a target protein,
wherein the target protein comprises two heterologous domains, wherein a first heterologous domain comprises a donor chromophore, and wherein a second heterologous domain comprises an acceptor chromophore,
wherein the donor chromophore and acceptor chromophore are a FRET pair, and
wherein the target protein is cell-associated;
b. contacting a sample comprising the target protein with a test compound to form a mixture;
c. measuring a fluorescence emission spectrum of the mixture during exposure to a light source,
wherein the measuring of the mixture occurs over a period of time no greater than 1 second;
d. decomposing the fluorescence emission spectrum into at least two component spectra,
wherein the component spectra comprise a donor chromophore emission and an acceptor chromophore emission;
e. calculating a ratio (R), wherein the coefficient of variation (CV) of R is no greater than 3%;
f. identifying whether the test compound present in the sample alters the FRET of the target protein,
wherein a difference of at least 1% between the R in the presence of the test compound and the R in the absence of the test compound indicates that the test compound alters the FRET of the target protein.

2. A method for identifying a compound that alters fluorescence resonance energy transfer (FRET) of a protein comprising:
a. providing a target protein and a second protein,
wherein the target protein comprises a first heterologous domain comprising a donor chromophore, wherein the second protein comprises a second heterologous domain comprising an acceptor chromophore, and wherein the donor chromophore and acceptor chromophore are a FRET pair, and
wherein the target protein is cell-associated;
b. contacting a sample comprising the target protein with a test compound to form a mixture;
c. measuring a fluorescence emission spectrum of the mixture during exposure to a light source,
wherein the measuring of the mixture occurs over a period of time no greater than 1 second;
d. decomposing the fluorescence emission spectrum into at least two component spectra,
wherein the component spectra comprise a donor chromophore emission and an acceptor chromophore emission;
e. calculating a ratio (R), wherein the coefficient of variation (CV) of R is no greater than 3%;
f. whether the test compound present in the sample alters the FRET of the target protein,
wherein a difference of at least 1% between the R in the presence of the test compound and the R in the absence of the test compound indicates that the test compound alters the FRET of the target protein.

3. The method of claim 1 wherein an altered FRET is the result of a change in structure of the target protein, a change in ligand-binding by the target protein, or a combination thereof.

4. The method of claim 1 wherein the target protein is present in a genetically engineered cell.

5. The method of claim 4 wherein the target protein is stably expressed by the genetically engineered cell.

6. The method of claim 1 wherein the target protein is present in a microsomal cellular preparation.

7. The method of claim 3 wherein the target protein and the second protein are present in a genetically engineered cell.

8. The method of claim 7 wherein the target protein and the second protein are stably expressed by the genetically engineered cell.

9. The method of claim 2 wherein the target protein and the second protein are present in a cell homogenate.

10. The method of claim 2 wherein the target protein and the second protein are present in a microsomal cellular preparation.

11. The method of claim 4 wherein the cell is in suspension.

12. The method of claim 1 wherein the donor chromophore is a green fluorescent protein and the acceptor chromophore is a red fluorescent protein.

13. The method of claim 1 wherein the donor chromophore is a cyan fluorescent protein and the acceptor chromophore is a yellow fluorescent protein.

14. The method of claim 1 wherein the fluorescence emission spectrum is decomposed into at least four component spectra,
wherein the component spectra comprise a donor chromophore emission and an acceptor chromophore emission, and further comprise a water Raman emission, and a cell autofluorescence emission.

15. The method of claim 14 wherein the fluorescence emission spectrum is decomposed according to
F_{Fit}(λ)=aF_{D}(λ)+bF_{A}(λ)+cF_{C}(λ)+dF_{W}(λ)

16. The method of claim 1 wherein R is determined according to
$$R = \frac{Acceptor emission}{Donor emission} = \frac{b {F}_{A}}{a {F}_{D}} ,$$

17. The method of claim 1 further comprising determining the FRET efficiency, wherein the FRET efficiency is determined according to
$$FRET = \frac{FR \times QR - AR}{1 + FR \times QR} .$$

18. The method of claim 1 adapted for use in a high-throughput format.

19. The method of claim 6 wherein the genetically engineered cell is a eukaryotic cell.

20. The method of claim 1 further comprising:
measuring a fluorescence lifetime of the donor chromophore; and
calculating the distance distributions and mole fractions of structural states of the target protein, wherein the distance distributions and mole fractions of structural states are calculated according to
$$\begin{matrix} {F}_{D} ( t ) = \text{?} {A}_{c} exp ( - 1 / \text{?} ) & ( Eq . 1 ) \\ {F}_{DA} ( t ) = \text{?} {X}_{j} \cdot {T}_{j} ( t ) & ( Eq . 2 ) \\ F ( t ) = {x}_{D} {F}_{D} ( t ) + {x}_{DA} {F}_{DA} ( t ) & ( Eq . 3 ) \\ {T}_{j} ( t ) = \text{?} {ρ}_{j} ( R ) - \text{?} A \text{?} exp ( \frac{- t}{\text{?}} \cdot [ 1 + ( \frac{\text{?}}{\text{?}} ) \text{?} ] ) dR & ( Eq . 4 ) \\ {ρ}_{j} ( R ) = \frac{1}{{a}_{j} \cdot \sqrt{2 π}} exp ( \frac{- [ {R\_R}_{j} ] \text{?}}{2 {σ}_{j}^{2}} ) & ( Eq . 5 ) \\ \text{?} = {FWHM}_{j} ( 2 \sqrt{2 ln 2} ) . \\ \text{?} \text{indicates text missing or illegible when filed} & ( Eq . 6 )\end{matrix}$$

21. A method for identifying a test compound as a potential false-positive, comprising:
calculating a similarity index (SI), wherein an SI of greater than one standard deviation of the normal distribution of all test compounds indicates a test compound is a fluorescent compound and a potential false-positive.

22. The method of claim 21 wherein SI is determined according to
$$SI = 1 - \frac{\text{?}}{\text{?}} . \\ \text{?} \text{indicates text missing or illegible when filed}$$

23. A computer-implemented method for use in analysis of fluorescence emission data comprising:
I. providing a dataset representative of fluorescence emission data obtained for use in analysis of interaction between a target protein and test compounds, wherein providing the dataset comprises:
a. providing a target protein,
wherein the target protein comprises two heterologous domains, wherein a first heterologous domain comprises a donor chromophore, and wherein a second heterologous domain comprises an acceptor chromophore,
wherein the donor chromophore and acceptor chromophore are a FRET pair;
b. contacting a plurality of samples comprising the target protein with test compounds to form a mixture, wherein each sample comprises a different test compound;
c. obtaining a fluorescence emission spectrum of each mixture during exposure to a light source, wherein the dataset comprises the fluorescence emission spectrum of each mixture;
II. decomposing each fluorescence emission spectrum of the dataset into at least two component spectra,
wherein the component spectra comprise a donor chromophore emission and an acceptor chromophore emission,
wherein the decomposing comprises fitting the component spectra to a linear model, determining the contribution of each signal, and using the shape of each component spectra to decompose the fluorescence spectrum;
III. calculating a ratio (R) for each decomposed fluorescence emission spectrum,
wherein calculating R comprises determining the total fluorescence from the acceptor chromophore and the total fluorescence from the donor chromophore,
wherein the coefficient of variation (CV) of R is no greater than 3%;
IV. identifying whether the test compound present in one of the samples alters the FRET of the target protein,
wherein a difference of at least 1% between the R in the presence of the test compound and the R in the absence of the test compound indicates that the test compound alters the FRET of the target protein.
